(19) United States
(12) Patent Application Publication
(10) Pub. No.: US 2022/0073485 A1
(43) Pub. Date: Mar. 10, 2022

(54) Functionalised Amine Derivatives as IL-17 Modulators

(71) Applicant: UCB Biopharma SRL, Brussels (BE)

(72) Inventors: Anne Marie Foley, Slough, Berkshire (GB); Jag Paul Heer, Slough, Berkshire (GB); Fabien Claude Lecomte, Slough, Berkshire (GB); Nathaniel Julius Thomas Monck, Abingdon, Oxforshire (GB); Matthew Duncan Selby, Slough, Berkshire (GB); Mengyang Xuan, Slough, Berkshire (GB); Lihu Yang, Cambridge, MA (US)

(21) Appl. No.: 17/299,137

(22) PCT Filed: Nov. 27, 2019
(86) PCT No.: PCT/EP2019/082779

§ 371 Date: Jun. 2, 2021

(30) Foreign Application Priority Data
Dec. 11, 2018 (GB) 1820166.5

(51) Int. Cl. C07D 309/04; C07D 413/14

(52) U.S. Cl. CPC C07D 309/04; C07D 413/14

(57) ABSTRACT

A series of functionalised amine derivatives of formula (I) as defined herein, being potent modulators of human IL-17 activity, are accordingly of benefit in the treatment and/or prevention of various human ailments, including inflammatory and autoimmune disorders.

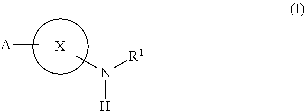

The present invention relates to pharmacologically active functionalised amine derivatives, and to their use in therapy. The compounds in accordance with the invention act as modulators of IL-17 activity, and are accordingly of benefit as pharmaceutical agents for the treatment and/or prevention of pathological conditions, including adverse inflammatory and autoimmune disorders.

IL-17A (originally named CTLA-8 and also known as IL-17) is a pro-inflammatory cytokine and the founder member of the IL-17 family (Rouvier et al., J. Immunol., 1993, 150, 5445-5456). Subsequently, five additional members of the family (IL-17B to IL-17F) have been identified, including the most closely related, IL-17F (ML-1), which shares approximately 55% amino acid sequence homology with IL-17A (Moseley et al., Cytokine Growth Factor Rev., 2003, 14, 155-174). IL-17A and IL-17F are expressed by the recently defined autoimmune related subset of T helper cells, Th17, that also express IL-21 and IL-22 signature cytokines (Korn et al., Ann. Rev. Immunol., 2009, 27, 485-517). IL-17A and IL-17F are expressed as homodimers, but may also be expressed as the IL-17A/F heterodimer (Wright et al., J. Immunol., 2008, 181, 2799-2805). IL-17A and F signal through the receptors IL-17R, IL-17RC or an IL-17RA/RC receptor complex (Gaffen, Cytokine, 2008, 43, 402-407). Both IL-17A and IL-17F have been associated with a number of autoimmune diseases.

The compounds in accordance with the present invention, being potent modulators of human IL-17 activity, are therefore beneficial in the treatment and/or prevention of various human ailments, including inflammatory and autoimmune disorders.

Furthermore, the compounds in accordance with the present invention may be beneficial as pharmacological standards for use in the development of new biological tests and in the search for new pharmacological agents. Thus, the compounds of this invention may be useful as radioligands in assays for detecting pharmacologically active compounds.

WO 2013/116682 and WO 2014/066726 relate to separate classes of chemical compounds that are stated to modulate the activity of IL-17 and to be useful in the treatment of medical conditions, including inflammatory diseases.

Co-pending international patent application PCT/EP2018/065558 (published on 20 Dec. 2018 as WO 2018/229079) describes spirocyclic oxoindoline derivatives, and analogues thereof, that are potent modulators of human IL-17 activity, and are therefore beneficial in the treatment of human ailments, including inflammatory and autoimmune disorders.

Co-pending international patent application PCT/EP2019/050594 (published on 18 Jul. 2019 as WO 2019/138017) describes substituted fused bicyclic imidazole derivatives, including benzimidazole derivatives and analogues thereof, that are potent modulators of human IL-17 activity, and are therefore beneficial in the treatment of human ailments, including inflammatory and autoimmune disorders.

None of the prior art available to date, however, discloses or suggests the precise structural class of functionalised amine derivatives as provided by the present invention.

The present invention provides a compound of formula (I) or an N-oxide thereof, or a pharmaceutically acceptable salt thereof:

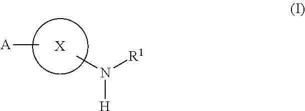

wherein

X represents an optionally substituted benzene ring; or an optionally substituted five-membered heteroaromatic ring selected from furyl, thienyl, pyrrolyl, pyrazolyl, oxazolyl, isoxazolyl, thiazolyl, isothiazolyl and imidazolyl; or an optionally substituted six-membered heteroaromatic ring selected from pyridinyl, pyridazinyl, pyrimidinyl and pyrazinyl;

A represents C₃₋₉ cycloalkyl, C₃₋₇ heterocycloalkyl or C₄₋₉ heterobicycloalkyl, any of which groups may be optionally substituted by one or more substituents;
R¹ represents —COR^{a} or —SO₂R^{b}; or R¹ represents C₁₋₆ alkyl, C₃₋₉ cycloalkyl, C₃₋₉ cycloalkyl(C₁₋₆)alkyl, C₅₋₉ spirocycloalkyl(C₁₋₆)alkyl, aryl, aryl(C₁₋₆)alkyl, C₃₋₇ heterocycloalkyl, C₃₋₇ heterocycloalkyl(C₁₋₆)alkyl, heteroaryl or heteroaryl(C₁₋₆)alkyl, any of which groups may be optionally substituted by one or more substituents;

R^{a} represents hydrogen; or R^{a} represents C₁₋₆ alkyl, C₂₋₇ alkenyl, C₃₋₉ cycloalkyl, C₃₋₉ cycloalkyl(C₁₋₆)alkyl, C₃₋₉ cycloalkylidenyl(C₁₋₆)alkyl, C₄₋₉ bicycloalkyl(C₁₋₆)alkyl, C₄₋₉ bicycloalkylidenyl(C₁₋₆)alkyl, C₅₋₉ spirocycloalkyl(C₁₋₆)alkyl, C₉-11 tricycloalkyl-(C₁₋₆)alkyl, aryl, aryl(C₁₋₆)alkyl, C₃₋₇ heterocycloalkyl, C₃₋₇ heterocycloalkyl(C₁₋₆)alkyl, C₃₋₇ heterocycloalkylidenyl(C₁₋₆)alkyl, heteroaryl or heteroaryl(C₁₋₆)alkyl, any of which groups may be optionally substituted by one or more substituents; and

R^{b} represents C₁₋₆ alkyl, C₂₋₇ alkenyl, C₃₋₉ cycloalkyl, C₃₋₉ cycloalkyl(C₁₋₆)alkyl, C₃₋₉ cycloalkylidenyl(C₁₋₆)alkyl, C₄₋₉ bicycloalkyl(C₁₋₆)alkyl, C₄₋₉ bicycloalkylidenyl-(C₁₋₆)alkyl, C₅₋₉ spirocycloalkyl(C₁₋₆)alkyl, C₉₋₁₁ tricycloalkyl(C₁₋₆)alkyl, aryl, aryl(C₁₋₆)-alkyl, C₃₋₇ heterocycloalkyl, C₃₋₇ heterocycloalkyl(C₁₋₆)alkyl, C₃₋₇ heterocycloalkylidenyl-(C₁₋₆)alkyl, heteroaryl or heteroaryl(C₁₋₆)alkyl, any of which groups may be optionally substituted by one or more substituents.

The present invention also provides a compound of formula (I) as defined above or an N-oxide thereof, or a pharmaceutically acceptable salt thereof, for use in therapy.

The present invention also provides a compound of formula (I) as defined above or an N-oxide thereof, or a pharmaceutically acceptable salt thereof, for use in the treatment and/or prevention of disorders for which the administration of a modulator of IL-17 function is indicated.

The present invention also provides the use of a compound of formula (I) as defined above or an N-oxide thereof, or a pharmaceutically acceptable salt thereof, for the manufacture of a medicament for the treatment and/or prevention of disorders for which the administration of a modulator of IL-17 function is indicated.

The present invention also provides a method for the treatment and/or prevention of disorders for which the administration of a modulator of IL-17 function is indicated which comprises administering to a patient in need of such treatment an effective amount of a compound of formula (I) as defined above or an N-oxide thereof, or a pharmaceutically acceptable salt thereof.

Where any of the groups in the compounds of formula (I) above is stated to be optionally substituted, this group may be unsubstituted, or substituted by one or more substituents. Typically, such groups will be unsubstituted, or substituted by one, two or three substituents. Suitably, such groups will be unsubstituted, or substituted by one or two substituents.

For use in medicine, the salts of the compounds of formula (I) will be pharmaceutically acceptable salts. Other salts may, however, be useful in the preparation of the compounds of formula (I) or of their pharmaceutically acceptable salts. Standard principles underlying the selection and preparation of pharmaceutically acceptable salts are described, for example, in Handbook of Pharmaceutical Salts: Properties, Selection and Use, ed. P. H. Stahl & C. G. Wermuth, Wiley-VCH, 2002. Suitable pharmaceutically acceptable salts of the compounds of formula (I) include acid addition salts which may, for example, be formed by mixing a solution of a compound of formula (I) with a solution of a pharmaceutically acceptable acid.

The present invention also includes within its scope co-crystals of the compounds of formula (I) above. The technical term “co-crystal” is used to describe the situation where neutral molecular components are present within a crystalline compound in a definite stoichiometric ratio. The preparation of pharmaceutical co-crystals enables modifications to be made to the crystalline form of an active pharmaceutical ingredient, which in turn can alter its physicochemical properties without compromising its intended biological activity (see Pharmaceutical Salts and Co-crystals, ed. J. Wouters & L. Quere, RSC Publishing, 2012).

Suitable alkyl groups which may be present on the compounds of use in the invention include straight-chained and branched C₁₋₆ alkyl groups, for example C₁₋₄ alkyl groups. Typical examples include methyl and ethyl groups, and straight-chained or branched propyl, butyl and pentyl groups. Particular alkyl groups include methyl, ethyl, n-propyl, isopropyl, n-butyl, sec-butyl, isobutyl, tert-butyl, 2,2-dimethylpropyl and 3-methylbutyl. Derived expressions such as “C₁₋₆ alkoxy”, “C₁₋₆ alkylthio”, “C₁₋₆ alkylsulphonyl” and “C₁₋₆ alkylamino” are to be construed accordingly.

Suitable alkenyl groups which may be present on the compounds of use in the invention include straight-chained and branched C₂₋₇ alkenyl groups, for example C₂₋₄ alkenyl groups. Typical examples include vinyl, allyl and buten-1-yl.

The term “C₃₋₉ cycloalkyl” as used herein refers to monovalent groups of 3 to 9 carbon atoms derived from a saturated monocyclic hydrocarbon, and may comprise benzo-fused analogues thereof. Suitable C₃₋₉ cycloalkyl groups include cyclopropyl, cyclobutyl, benzocyclobutenyl, cyclopentyl, indanyl, cyclohexyl, tetrahydronaphthalenyl, cycloheptyl, benzocycloheptenyl, cyclooctyl and cyclononanyl.

The term “C₃₋₉ cycloalkylidenyl” as used herein refers to monovalent groups of 3 to 9 carbon atoms derived from a saturated monocyclic hydrocarbon, optionally comprising benzo-fused analogues thereof, attached to the remainder of the molecule via a C═C double bond. Typically, such groups include cyclobutylidenyl, cyclopentylidenyl, cyclohexylidenyl, cycloheptylidenyl, cyclooctylidenyl and cyclononanylidenyl.

The term “C₄₋₉ bicycloalkyl” as used herein refers to monovalent groups of 4 to 9 carbon atoms derived from a saturated bicyclic hydrocarbon. Typical bicycloalkyl groups include bicyclo[1.1.1]pentanyl, bicyclo[3.1.0]hexanyl, bicyclo[4.1.0]heptanyl, bicyclo-[2.2.1]heptanyl, bicyclo[2.2.2]octanyl, bicyclo[3.3.0]octanyl and bicyclo[3.2.1]octanyl.

The term “C₄₋₉ bicycloalkylidenyl” as used herein refers to monovalent groups of 4 to 9 carbon atoms derived from a saturated bicyclic hydrocarbon, attached to the remainder of the molecule via a C═C double bond. Typically, such groups include bicyclo[3.1.0]hexanylidenyl, bicyclo[2.2.1]heptanylidenyl and bicyclo[3.2.1]octanyliden-yl.

The term “C₅₋₉ spirocycloalkyl” as used herein refers to saturated bicyclic ring systems containing 5 to 9 carbon atoms, in which the two rings are linked by a common atom. Suitable spirocycloalkyl groups include spiro[2.3]hexanyl, spiro[2.4]heptanyl, spiro[3.3]heptanyl, spiro[3.4]octanyl, spiro[3.5]nonanyl and spiro[4.4]nonanyl.

The term “C₉₋₁₁ tricycloalkyl” as used herein refers to monovalent groups of 9 to 11 carbon atoms derived from a saturated tricyclic hydrocarbon. Typical tricycloalkyl groups include adamantanyl.

The term “aryl” as used herein refers to monovalent carbocyclic aromatic groups derived from a single aromatic ring or multiple condensed aromatic rings. Suitable aryl groups include phenyl and naphthyl, preferably phenyl.

Suitable aryl(C₁₋₆)alkyl groups include benzyl, phenylethyl, phenylpropyl and naphthylmethyl.

The term “C₃₋₇ heterocycloalkyl” as used herein refers to saturated monocyclic rings containing 3 to 7 carbon atoms and at least one heteroatom selected from oxygen, sulphur and nitrogen, and may comprise benzo-fused analogues thereof. Suitable heterocycloalkyl groups include oxetanyl, azetidinyl, tetrahydrofuranyl, dihydrobenzofuranyl, dihydrobenzothienyl, pyrrolidinyl, indolinyl, isoindolinyl, oxazolidinyl, thiazolidinyl, isothiazolidinyl, imidazolidinyl, tetrahydropyranyl, chromanyl, tetrahydrothiopyranyl, piperidinyl, 1,2,3,4-tetrahydroquinolinyl, 1,2,3,4-tetrahydroisoquinolinyl, piperazinyl, 1,2,3,4-tetrahydroquinoxalinyl, hexahydro-[1,2,5]thiadiazolo[2,3-a]pyrazinyl, homopiperazinyl, morpholinyl, benzoxazinyl, thiomorpholinyl, azepanyl, oxazepanyl, diazepanyl, thiadiazepanyl and azocanyl.

The term “C₃₋₇ heterocycloalkylidenyl” as used herein refers to saturated monocyclic rings containing 3 to 7 carbon atoms and at least one heteroatom selected from oxygen, sulphur and nitrogen, attached to the remainder of the molecule via a C═C double bond. Typically, such groups include tetrahydropyranylidenyl and piperidinylidenyl.

The term “C₄₋₉ heterobicycloalkyl” as used herein corresponds to C₄₋₉ bicycloalkyl wherein one or more of the carbon atoms have been replaced by one or more heteroatoms selected from oxygen, sulphur and nitrogen. Typical heterobicycloalkyl groups include 6-oxabicyclo[3.1.0]hexanyl, 3-azabicyclo[3.1.0]hexanyl, 2-oxa-5-azabicyclo[2.2.1]heptanyl, 6-azabicyclo[3.2.0]heptanyl, 6-oxabicyclo[3.1.1]heptanyl, 3-azabicyclo[3.1.1]heptanyl, 3-azabicyclo[4.1.0]heptanyl, 2-oxabicyclo[2.2.2]octanyl, quinuclidinyl, 2-oxa-5-azabicyclo-[2.2.2]octanyl, 8-oxabicyclo[3.2.1]octanyl, 3-azabicyclo[3.2.1]octanyl, 8-azabicyclo-[3.2.1]octanyl, 3-oxa-8-azabicyclo[3.2.1]octanyl, 3,8-diazabicyclo[3.2.1]octanyl, 3,6-diazabicyclo[3.2.2]nonanyl, 3-oxa-7-azabicyclo[3.3.1]nonanyl, 3,7-dioxa-9-azabicyclo-[3.3.1]nonanyl and 3,9-diazabicyclo[4.2.1]nonanyl.

The term “heteroaryl” as used herein refers to monovalent aromatic groups containing at least 5 atoms derived from a single ring or multiple condensed rings, wherein one or more carbon atoms have been replaced by one or more heteroatoms selected from oxygen, sulphur and nitrogen. Suitable heteroaryl groups include furyl, benzofuryl, dibenzofuryl, thienyl, benzothienyl, thieno[2,3-c]pyrazolyl, thieno[3,4-b][1,4]dioxinyl, dibenzothienyl, pyrrolyl, indolyl, pyrrolo[2,3-b]pyridinyl, pyrrolo[3,2-c]pyridinyl, pyrrolo[3,4-b]pyridinyl, pyrazolyl, pyrazolo[1,5-a]pyridinyl, pyrazolo[3,4-d]pyrimidinyl, pyrazolo[1,5-a]pyrazinyl, indazolyl, 4,5,6,7-tetrahydroindazolyl, oxazolyl, benzoxazolyl, isoxazolyl, thiazolyl, benzothiazolyl, isothiazolyl, imidazolyl, benzimidazolyl, imidazo-[2,1-b]thiazolyl, imidazo[1,2-a]pyridinyl, imidazo[4,5-b]pyridinyl, imidazo[1,2-b]-pyridazinyl, purinyl, imidazo[1,2-a]pyrimidinyl, imidazo[1,2-a]pyrazinyl, oxadiazolyl, thiadiazolyl, triazolyl, [1,2,4]triazolo[1,5-a]pyrimidinyl, benzotriazolyl, tetrazolyl, pyridinyl, quinolinyl, isoquinolinyl, naphthyridinyl, pyridazinyl, cinnolinyl, phthalazinyl, pyrimidinyl, quinazolinyl, pyrazinyl, quinoxalinyl, pteridinyl, triazinyl and chromenyl groups.

The term “halogen” as used herein is intended to include fluorine, chlorine, bromine and iodine atoms, typically fluorine, chlorine or bromine.

Where the compounds of formula (I) have one or more asymmetric centres, they may accordingly exist as enantiomers. Where the compounds in accordance with the invention possess two or more asymmetric centres, they may additionally exist as diastereomers. The invention is to be understood to extend to the use of all such enantiomers and diastereomers, and to mixtures thereof in any proportion, including racemates. Formula (I) and the formulae depicted hereinafter are intended to represent all individual stereoisomers and all possible mixtures thereof, unless stated or shown otherwise. In addition, compounds of formula (I) may exist as tautomers, for example keto (CH₂C═O)↔enol (CH═CHOH) tautomers or amide (NHC═O)↔hydroxyimine (N═COH) tautomers. Formula (I) and the formulae depicted hereinafter are intended to represent all individual tautomers and all possible mixtures thereof, unless stated or shown otherwise.

It is to be understood that each individual atom present in formula (I), or in the formulae depicted hereinafter, may in fact be present in the form of any of its naturally occurring isotopes, with the most abundant isotope(s) being preferred. Thus, by way of example, each individual hydrogen atom present in formula (I), or in the formulae depicted hereinafter, may be present as a ¹H, ²H (deuterium) or ³H (tritium) atom, preferably ¹H. Similarly, by way of example, each individual carbon atom present in formula (I), or in the formulae depicted hereinafter, may be present as a ¹²C, ¹³C ¹⁴C atom, preferably ¹²C.

In a first embodiment, X represents an optionally substituted benzene ring.

In a second embodiment, X represents an optionally substituted five-membered heteroaromatic ring selected from furyl, thienyl, pyrrolyl, pyrazolyl, oxazolyl, isoxazolyl, thiazolyl, isothiazolyl and imidazolyl. In a subset of that embodiment, X represents pyrazolyl, isoxazolyl or thiazolyl, any of which groups may be optionally substituted by one or, where possible, two substituents in addition to A and —NHR¹. In a first aspect of that embodiment, X represents pyrazolyl, which group may be optionally substituted by one or two substituents in addition to A and —NHR¹. In a second aspect of that embodiment, X represents isoxazolyl, which group may be optionally substituted by one substituent in addition to A and —NHR¹. In a third aspect of that embodiment, X represents thiazolyl, which group may be optionally substituted by one substituent in addition to A and —NHR¹.

In a third embodiment, X represents an optionally substituted six-membered heteroaromatic ring selected from pyridinyl, pyridazinyl, pyrimidinyl and pyrazinyl. In a particular aspect of that embodiment, X represents pyridinyl, which group may be optionally substituted by one, two or three substituents in addition to A and —NHR¹.

Suitably, X represents an optionally substituted benzene ring; or an optionally substituted five-membered heteroaromatic ring selected from pyrazolyl, isoxazolyl and thiazolyl; or an optionally substituted six-membered heteroaromatic ring selected from pyridinyl.

The aromatic or heteroaromatic ring X is substituted by A and —NHR¹, and may optionally be substituted, where possible, by one or more additional substituents. Generally, X may be substituted, where possible, by one, two, three or four additional substituents; suitably by one, two or three additional substituents; typically by one or two additional substituents. In a first embodiment, X is substituted by A and —NHR¹, and by no additional substituents. In a second embodiment, X is substituted by A and —NHR¹, and by one additional substituent. In a third embodiment, X is substituted by A and —NHR¹, and by two additional substituents. In a fourth embodiment, X is substituted by A and —NHR¹, and by three additional substituents. In a fifth embodiment, X is substituted by A and —NHR¹, and by four additional substituents.

Typical examples of optional substituents on X include one, two or three substituents independently selected from halogen, cyano, C₁₋₆ alkyl, fluoromethyl, difluoromethyl, trifluoromethyl, hydroxy, C₁₋₆ alkoxy, difluoromethoxy, trifluoromethoxy, C₁₋₆ alkylthio, C₁₋₆ alkylsulfinyl, C₁₋₆ alkylsulfonyl, amino, C₁₋₆ alkylamino, di(C₁₋₆)alkylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl and di(C₁₋₆)alkylaminosulfonyl.

Suitable examples of optional substituents on X include one, two or three substituents independently selected from halogen, C₁₋₆ alkyl and C₁₋₆ alkoxy.

Typical examples of particular substituents on X include one, two or three substituents independently selected from fluoro, chloro, bromo, cyano, methyl, fluoromethyl, difluoromethyl, trifluoromethyl, hydroxy, methoxy, difluoromethoxy, trifluoromethoxy, methylthio, methylsulfinyl, methylsulfonyl, amino, methylamino, dimethylamino, formyl, acetyl, carboxy, methoxycarbonyl, ethoxycarbonyl, tert-butoxycarbonyl, aminocarbonyl, methylaminocarbonyl, dimethylaminocarbonyl, aminosulfonyl, methylaminosulfonyl and dimethylaminosulfonyl.

Suitable examples of particular substituents on X include one, two or three substituents independently selected from fluoro, chloro, bromo, methyl and methoxy.

In a first embodiment, integer A represents optionally substituted C₃₋₉ cycloalkyl. In one aspect of that embodiment, A represents optionally substituted C₄₋₇ cycloalkyl.

In a second embodiment, integer A represents optionally substituted C₃₋₇ heterocycloalkyl. In one aspect of that embodiment, A represents optionally substituted C₄₋₆ heterocycloalkyl.

In a third embodiment, integer A represents optionally substituted C₄₋₉ heterobicycloalkyl. In one aspect of that embodiment, A represents optionally substituted C₅₋₇ heterobicycloalkyl.

Typically, integer A represents cyclopropyl, cyclobutyl, cyclopentyl, cyclohexyl, cycloheptyl, cyclooctyl, cyclononanyl, oxetanyl, azetidinyl, tetrahydrofuranyl, pyrrolidinyl, oxazolidinyl, thiazolidinyl, isothiazolidinyl, imidazolidinyl, tetrahydropyranyl, tetrahydrothiopyranyl, piperidinyl, piperazinyl, homopiperazinyl, morpholinyl, thiomorpholinyl, azepanyl, oxazepanyl, diazepanyl, thiadiazepanyl, azocanyl, 6-oxabicyclo[3.1.0]hexanyl, 6-oxabicyclo[3.1.1]heptanyl or 8-oxabicyclo[3.2.1]octanyl, any of which groups may be optionally substituted by one or more substituents.

Appositely, integer A represents tetrahydrofuranyl, pyrrolidinyl, tetrahydropyranyl or morpholinyl, any of which groups may be optionally substituted by one or more substituents.

Suitably, integer A represents tetrahydropyranyl or morpholinyl, either of which groups may be optionally substituted by one or more substituents.

Typical examples of optional substituents on integer A include one, two or three substituents independently selected from C₁₋₆ alkyl, halogen, cyano, trifluoromethyl, hydroxy, hydroxy(C₁₋₆)alkyl, oxo, C₁₋₆ alkoxy, C₁₋₆ alkylthio, C₁₋₆ alkylsulfinyl, C₁₋₆ alkylsulfonyl, C₂₋₆ alkylcarbonyl, amino, imino, C₁₋₆ alkylamino, di(C₁₋₆)alkylamino, aminocarbonyl, C₁₋₆ alkylaminocarbonyl and di(C₁₋₆)alkylaminocarbonyl. Additional examples include difluoroazetidinylcarbonyl.

Selected examples of optional substituents on integer A include one, two or three substituents independently selected from cyano, hydroxy, hydroxy(C₁₋₆)alkyl, oxo, C₁₋₆ alkoxy, di(C₁₋₆)alkylaminocarbonyl and difluoroazetidinylcarbonyl.

Suitable examples of optional substituents on integer A include one, two or three substituents independently selected from cyano, hydroxy, hydroxy(C₁₋₆)alkyl, oxo, C₁₋₆ alkoxy and di(C₁₋₆)alkylaminocarbonyl.

Typical examples of particular substituents on integer A include one, two or three substituents independently selected from methyl, fluoro, chloro, bromo, cyano, trifluoromethyl, hydroxy, hydroxymethyl, oxo, methoxy, methylthio, methylsulfinyl, methylsulfonyl, acetyl, amino, imino, methylamino, dimethylamino, aminocarbonyl, methylaminocarbonyl and dimethylaminocarbonyl. Additional examples include difluoroazetidinylcarbonyl.

Selected examples of particular substituents on integer A include one, two or three substituents independently selected from cyano, hydroxy, hydroxymethyl, oxo, methoxy, dimethylaminocarbonyl and difluoroazetidinylcarbonyl.

Suitable examples of particular substituents on integer A include one, two or three substituents independently selected from cyano, hydroxy, hydroxymethyl, oxo, methoxy and dimethylaminocarbonyl.

Selected values of integer A include tetrahydrofuranyl, oxopyrrolidinyl, tetrahydropyranyl, cyanotetrahydropyranyl, hydroxytetrahydropyranyl, hydroxymethyl-tetrahydropyranyl, methoxytetrahydropyranyl, dimethylaminocarbonyltetrahydropyranyl, difluoroazetidinylcarbonyltetrahydropyranyl and morpholinyl.

Typical values of integer A include tetrahydrofuranyl, oxopyrrolidinyl, tetrahydropyranyl, cyanotetrahydropyranyl, hydroxytetrahydropyranyl, hydroxymethyl-tetrahydropyranyl, methoxytetrahydropyranyl, dimethylaminocarbonyltetrahydropyranyl and morpholinyl.

Typical examples of optional substituents on R¹ include one, two or three substituents independently selected from C₁₋₆ alkyl, halogen, cyano, trifluoromethyl, hydroxy, C₁₋₆ alkoxy, C₁₋₆ alkylthio, C₁₋₆ alkylsulphinyl, C₁₋₆ alkylsulphonyl, C₂₋₆ alkylcarbonyl, amino, C₁₋₆ alkylamino and di(C₁₋₆)alkylamino.

Typical examples of particular substituents on R¹ include one, two or three substituents independently selected from methyl, fluoro, chloro, bromo, cyano, trifluoromethyl, hydroxy, oxo, methoxy, methylthio, methylsulphinyl, methylsulphonyl, acetyl, amino, methylamino and dimethylamino.

Suitably, R¹ represents —COR^{a}.

In a particular embodiment, R^{a} is other than hydrogen.

Typically, R^{a} represents C₃₋₉ cycloalkyl(C₁₋₆)alkyl or C₃₋₉ cycloalkylidenyl(C₁₋₆)-alkyl, either of which groups may be optionally substituted by one or more substituents.

Suitably, R^{a} represents C₃₋₉ cycloalkyl(C₁₋₆)alkyl, which group may be optionally substituted by one or more substituents.

Typical values of R^{a} include cyclohexylmethyl, cyclooctylmethyl and benzocyclobutylidenylmethyl, any of which groups may be optionally substituted by one or more substituents.

Suitable values of R^{a} include cyclohexylmethyl and cyclooctylmethyl, either of which groups may be optionally substituted by one or more substituents.

Favoured examples of optional substituents on R^{a} include one, two or three substituents independently selected from halogen, cyano, nitro, C₁₋₆ alkyl, trifluoromethyl, trifluoroethyl, phenyl, hydroxy, oxo, C₁₋₆ alkoxy, difluoromethoxy, trifluoromethoxy, C₁₋₆ alkylthio, C₁₋₆ alkylsulfinyl, C₁₋₆ alkylsulfonyl, amino, C₁₋₆ alkylamino, di(C₁₋₆)alkylamino, C₂₋₆ alkylcarbonylamino, C₂₋₆ alkoxycarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl, di(C₁₋₆)alkylaminosulfonyl, —R^{5a}, —NHCOR⁶, —NHS(O)₂R⁶, —R⁷, —NHR⁷ and —CONHR⁷, wherein R^{5a}, R⁶ and R⁷ are as defined below.

Selected examples of optional substituents on R^{a} include one, two or three substituents independently selected from halogen, C₁₋₆ alkyl and —NHCOR⁶, wherein R⁶ is as defined below.

Favoured examples of specific substituents on R^{a} include one, two or three substituents independently selected from fluoro, chloro, bromo, cyano, nitro, methyl, ethyl, isopropyl, tert-butyl, trifluoromethyl, trifluoroethyl, phenyl, hydroxy, oxo, methoxy, isopropoxy, tert-butoxy, difluoromethoxy, trifluoromethoxy, methylthio, methylsulfinyl, methylsulfonyl, amino, methylamino, tert-butylamino, dimethylamino, acetylamino, methoxycarbonylamino, methylsulfonylamino, formyl, acetyl, carboxy, methoxycarbonyl, ethoxycarbonyl, tert-butoxycarbonyl, aminocarbonyl, methylaminocarbonyl, dimethylaminocarbonyl, aminosulfonyl, methylaminosulfonyl, dimethylaminosulfonyl, —R^{5a}, —NHCOR⁶, —NHS(O)₂R⁶, —R⁷, —NHR⁷ and —CONHR⁷, wherein R^{5a}, R⁶ and R⁷ are as defined below.

Selected examples of specific substituents on R^{a} include one, two or three substituents independently selected from chloro, methyl and —NHCOR⁶, wherein R⁶ is as defined below.

Typically, R^{b} represents C₃₋₉ cycloalkyl(C₁₋₆)alkyl or C₃₋₉ cycloalkylidenyl(C₁₋₆)-alkyl, either of which groups may be optionally substituted by one or more substituents.

Suitable values of R^{b} include cyclohexylmethyl, cyclooctylmethyl and benzocyclobutylidenylmethyl, any of which groups may be optionally substituted by one or more substituents.

Favoured examples of optional substituents on R^{b} include one, two or three substituents independently selected from halogen, cyano, nitro, C₁₋₆ alkyl, trifluoromethyl, trifluoroethyl, phenyl, hydroxy, oxo, C₁₋₆ alkoxy, difluoromethoxy, trifluoromethoxy, C₁₋₆ alkylthio, C₁₋₆ alkylsulfinyl, C₁₋₆ alkylsulfonyl, amino, C₁₋₆ alkylamino, di(C₁₋₆)alkylamino, C₂₋₆ alkylcarbonylamino, C₂₋₆ alkoxycarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl, di(C₁₋₆)alkylaminosulfonyl, —R^{5a}, —NHCOR⁶, —NHS(O)₂R⁶, —R⁷, —NHR⁷ and —CONHR⁷, wherein R^{5a}, R⁶ and R⁷ are as defined below.

Selected examples of optional substituents on R^{b} include one, two or three substituents independently selected from halogen, C₁₋₆ alkyl and —NHCOR⁶, wherein R⁶ is as defined below.

Favoured examples of specific substituents on R^{b} include one, two or three substituents independently selected from fluoro, chloro, bromo, cyano, nitro, methyl, ethyl, isopropyl, tert-butyl, trifluoromethyl, trifluoroethyl, phenyl, hydroxy, oxo, methoxy, isopropoxy, tert-butoxy, difluoromethoxy, trifluoromethoxy, methylthio, methylsulfinyl, methylsulfonyl, amino, methylamino, tert-butylamino, dimethylamino, acetylamino, methoxycarbonylamino, methylsulfonylamino, formyl, acetyl, carboxy, methoxycarbonyl, ethoxycarbonyl, tert-butoxycarbonyl, aminocarbonyl, methylaminocarbonyl, dimethylaminocarbonyl, aminosulfonyl, methylaminosulfonyl, dimethylaminosulfonyl, —R^{5a}, —NHCOR⁶, —NHS(O)₂R⁶, —R⁷, —NHR⁷ and —CONHR⁷, wherein R^{5a}, R⁶ and R⁷ are as defined below.

Selected examples of specific substituents on R^{b} include one, two or three substituents independently selected from chloro, methyl and —NHCOR⁶, wherein R⁶ is as defined below.

A particular sub-class of compounds according to the invention is represented by the compounds of formula (IA) and N-oxides thereof, and pharmaceutically acceptable salts thereof:

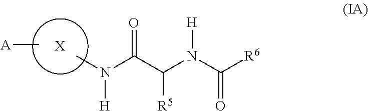

wherein

X and A are as defined above;

R⁵ represents hydrogen; or R⁵ represents C₁₋₅ alkyl, C₃₋₉ cycloalkyl, C₃₋₉ cyclo-alkyl(C₁₋₅)alkyl, C₄₋₉ bicycloalkyl, C₄₋₉ bicycloalkyl(C₁₋₅)alkyl, C₅₋₉ spirocycloalkyl, C₅₋₉ spirocycloalkyl(C₁₋₅)alkyl, C₉-11 tricycloalkyl, C₉-11 tricycloalkyl(C₁₋₅)alkyl, aryl, aryl-(C₁₋₅)alkyl, C₃₋₇ heterocycloalkyl, C₃₋₇ heterocycloalkyl(C₁₋₅)alkyl, heteroaryl or heteroaryl(C₁₋₅)alkyl, any of which groups may be optionally substituted by one or more substituents;
R⁶ represents —NR^{6a}R^{6b} or —OR^{6c}; or R⁶ represents C₁₋₉ alkyl, C₃₋₉ cycloalkyl, C₃₋₉ cycloalkyl(C₁₋₆)alkyl, aryl, aryl(C₁₋₆)alkyl, C₃₋₇ heterocycloalkyl, C₃₋₇ heterocycloalkyl-(C₁₋₆)alkyl, heteroaryl, heteroaryl(C₁₋₆)alkyl or spiro[(C₃₋₇)heterocycloalkyl][heteroaryl], any of which groups may be optionally substituted by one or more substituents;

R^{6a} represents hydrogen; or R^{6a} represents C₁₋₆ alkyl, C₃₋₇ cycloalkyl, C₃₋₇ cyclo-alkyl(C₁₋₆)alkyl, aryl, aryl(C₁₋₆)alkyl, C₃₋₇ heterocycloalkyl, C₃₋₇ heterocycloalkyl(C₁₋₆)-alkyl, heteroaryl, heteroaryl(C₁₋₆)alkyl or spiro[(C₃₋₇)heterocycloalkyl][heteroaryl], any of which groups may be optionally substituted by one or more substituents;

R^{6b} represents hydrogen or C₁₋₆ alkyl; and

R^{6c} represents C₁₋₆ alkyl, C₃₋₇ cycloalkyl, C₃₋₇ cycloalkyl(C₁₋₆)alkyl, aryl, aryl(C₁₋₆)alkyl, C₃₋₇ heterocycloalkyl, C₃₋₇ heterocycloalkyl(C₁₋₆)alkyl, heteroaryl or heteroaryl(C₁₋₆)alkyl, any of which groups may be optionally substituted by one or more substituents.

A second sub-class of compounds according to the invention is represented by the compounds of formula (IB) and N-oxides thereof, and pharmaceutically acceptable salts thereof:

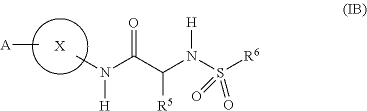

wherein

X, A, R⁵ and R⁶ are as defined above.

A third sub-class of compounds according to the invention is represented by the compounds of formula (IC) and N-oxides thereof, and pharmaceutically acceptable salts thereof:

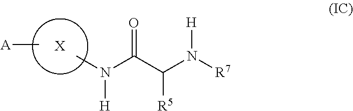

wherein

X, A and R⁵ are as defined above; and

R⁷ represents aryl, heteroaryl or spiro[(C₃₋₇)heterocycloalkyl][heteroaryl], any of which groups may be optionally substituted by one or more substituents.

A fourth sub-class of compounds according to the invention is represented by the compounds of formula (ID) and N-oxides thereof, and pharmaceutically acceptable salts thereof:

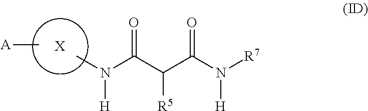

wherein

X, A, R⁵ and R⁷ are as defined above.

A fifth sub-class of compounds according to the invention is represented by the compounds of formula (IE) and N-oxides thereof, and pharmaceutically acceptable salts thereof:

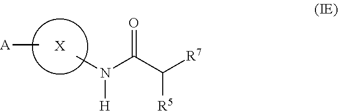

wherein

X, A, R⁵ and R⁷ are as defined above.

A sixth sub-class of compounds according to the invention is represented by the compounds of formula (IF) and N-oxides thereof, and pharmaceutically acceptable salts thereof:

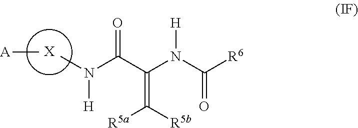

wherein

X, A and R⁶ are as defined above;

R^{5a} represents C₃₋₇ cycloalkyl, C₄₋₉ bicycloalkyl, aryl, C₃₋₇ heterocycloalkyl or heteroaryl, any of which groups may be optionally substituted by one or more substituents; and

R^{5b} represents hydrogen or C₁₋₆ alkyl; or

R^{5a} and R^{5b}, when taken together with the carbon atom to which they are both attached, represent C₃₋₇ cycloalkyl, C₄₋₉ bicycloalkyl or C₃₋₇ heterocycloalkyl, any of which groups may be optionally substituted by one or more substituents.

Typically, R⁵ represents hydrogen; or R⁵ represents C₁₋₅ alkyl, C₃₋₉ cycloalkyl, C₃₋₉ cycloalkyl(C₁₋₅)alkyl, C₄₋₉ bicycloalkyl, C₄₋₉ bicycloalkyl(C₁₋₅)alkyl, C₅₋₉ spirocycloalkyl, C₉₋₁₁ tricycloalkyl, C₉-11 tricycloalkyl(C₁₋₅)alkyl, aryl, aryl(C₁₋₅)alkyl, C₃₋₇ heterocycloalkyl, C₃₋₇ heterocycloalkyl(C₁₋₅)alkyl or heteroaryl(C₁₋₅)alkyl, any of which groups may be optionally substituted by one or more substituents.

Suitably, R⁵ represents C₃₋₉ cycloalkyl, which group may be optionally substituted by one or more substituents.

In a first embodiment, R⁵ represents hydrogen. In a second embodiment, R⁵ represents optionally substituted C₁₋₅ alkyl. In a third embodiment, R⁵ represents optionally substituted C₃₋₉ cycloalkyl. In a fourth embodiment, R⁵ represents optionally substituted C₃₋₉ cycloalkyl(C₁₋₅)alkyl. In a fifth embodiment, R⁵ represents optionally substituted C₄₋₉ bicycloalkyl. In a sixth embodiment, R⁵ represents optionally substituted C₄₋₉ bicycloalkyl(C₁₋₅)alkyl. In a seventh embodiment, R⁵ represents optionally substituted C₅₋₉ spirocycloalkyl. In an eighth embodiment, R⁵ represents optionally substituted C₅₋₉ spirocycloalkyl(C₁₋₅)alkyl. In a ninth embodiment, R⁵ represents optionally substituted C₉₋₁₁ tricycloalkyl. In a tenth embodiment, R⁵ represents optionally substituted C₉₋₁₁ tricycloalkyl(C₁₋₅)alkyl. In an eleventh embodiment, R⁵ represents optionally substituted aryl. In a twelfth embodiment, R⁵ represents optionally substituted aryl(C₁₋₅)alkyl. In a thirteenth embodiment, R⁵ represents optionally substituted C₃₋₇ heterocycloalkyl. In a fourteenth embodiment, R⁵ represents optionally substituted C₃₋₇ heterocycloalkyl(C₁₋₅)alkyl. In a fifteenth embodiment, R⁵ represents optionally substituted heteroaryl. In a sixteenth embodiment, R⁵ represents optionally substituted heteroaryl(C₁₋₅)alkyl.

In a particular embodiment, R⁵ is other than hydrogen.

Typical values of R⁵ include methyl, cyclobutyl, benzocyclobutenyl, cyclopentyl, indanyl, cyclohexyl, tetrahydronaphthalenyl, cycloheptyl, benzocycloheptenyl, cyclooctyl, cyclononanyl, cyclobutylmethyl, cyclobutylethyl, bicyclo[3.1.0]hexanyl, bicyclo[2.2.1]-heptanyl, bicyclo[3.3.0]octanyl, bicyclo[3.2.1]octanyl, bicyclo[1.1.1]pentanylmethyl, spiro[3.3]heptanyl, adamantanyl, adamantanylmethyl, phenyl, benzyl, phenylethyl, phenylpropyl, tetrahydropyranyl, azocanyl, dihydrobenzofuranylmethyl and pyrrolylethyl, any of which groups may be optionally substituted by one or more substituents.

Suitable values of R⁵ include cyclohexyl and cyclooctyl, either of which groups may be optionally substituted by one or more substituents.

Typical examples of optional substituents on R⁵ include one, two or three substituents independently selected from halogen, cyano, nitro, C₁₋₆ alkyl, trifluoromethyl, trifluoroethyl, phenyl, hydroxy, oxo, C₁₋₆ alkoxy, difluoromethoxy, trifluoromethoxy, C₁₋₆ alkylthio, C₁₋₆ alkylsulfinyl, C₁₋₆ alkylsulfonyl, amino, C₁₋₆ alkylamino, di(C₁₋₆)alkylamino, C₂₋₆ alkylcarbonylamino, C₂₋₆ alkoxycarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl and di(C₁₋₆)alkylaminosulfonyl.

Suitable examples of optional substituents on R⁵ include one, two or three substituents independently selected from halogen, cyano, C₁₋₆ alkyl, trifluoromethyl, phenyl, hydroxy, C₁₋₆ alkoxy and aminocarbonyl, especially C₁₋₆ alkyl.

Typical examples of specific substituents on R⁵ include one, two or three substituents independently selected from fluoro, chloro, bromo, cyano, nitro, methyl, ethyl, isopropyl, tert-butyl, trifluoromethyl, trifluoroethyl, phenyl, hydroxy, oxo, methoxy, isopropoxy, tert-butoxy, difluoromethoxy, trifluoromethoxy, methylthio, methylsulfinyl, methylsulfonyl, amino, methylamino, tert-butylamino, dimethylamino, acetylamino, methoxycarbonylamino, methylsulfonylamino, formyl, acetyl, carboxy, methoxycarbonyl, ethoxycarbonyl, tert-butoxycarbonyl, aminocarbonyl, methylaminocarbonyl, dimethylaminocarbonyl, aminosulfonyl, methylaminosulfonyl and dimethylaminosulfonyl.

Suitable examples of specific substituents on R⁵ include one, two or three substituents independently selected from fluoro, chloro, bromo, cyano, methyl, trifluoromethyl, phenyl, hydroxy, methoxy, isopropoxy. tert-butoxy and aminocarbonyl, especially methyl.

Apposite values of R⁵ include hydrogen, tert-butoxymethylcyclobutyl, methylcyclobutyl, dimethylcyclobutyl, phenylcyclobutyl, benzocyclobutenyl, cyclopentyl, methylcyclopentyl, indanyl, cyclohexyl, difluorocyclohexyl, methylcyclohexyl, dimethylcyclohexyl, trifluoromethylcyclohexyl, tetrahydronaphthalenyl, cycloheptyl, benzocycloheptenyl, cyclooctyl, cyclononanyl, cyclobutylmethyl, difluorocyclobutyl-methyl, dimethylcyclobutylmethyl, cyclobutylethyl, bicyclo[3.1.0]hexanyl, bicyclo[2.2.1]-heptanyl, bicyclo[3.3.0]octanyl, bicyclo[3.2.1]octanyl, bicyclo[1.1.1]pentanylmethyl, spiro[3.3]heptanyl, adamantanyl, adamantanylmethyl, (chloro)(fluoro)phenyl, (fluoro)-(methyl)phenyl, fluorobenzyl, chlorobenzyl, (chloro)(fluoro)benzyl, (bromo)(chloro)-benzyl, (chloro)(isopropoxy)benzyl, phenylethyl, chlorophenylethyl, phenylpropyl, tetrahydropyranyl, tetramethyltetrahydropyranyl, azocanyl, dihydrobenzofuranylmethyl and methylpyrrolylethyl.

Selected values of R⁵ include cyclohexyl, methylcyclohexyl and cyclooctyl.

Favoured values of R⁵ include methylcyclohexyl and cyclooctyl.

In a first embodiment, R⁵ represents methylcyclohexyl (especially 4-methyl-cyclohexyl). In a second embodiment, R⁵ represents cyclooctyl. In a third embodiment, R⁵ represents cyclohexyl.

In a first embodiment, R^{5a} represents optionally substituted C₃₋₇ cycloalkyl. In a second embodiment, R^{5a} represents optionally substituted C₄₋₉ bicycloalkyl. In a third embodiment, R^{5a} represents optionally substituted aryl. In a fourth embodiment, R^{5a} represents optionally substituted C₃₋₇ heterocycloalkyl. In a fifth embodiment, R^{5a} represents optionally substituted heteroaryl.

Typical values of R^{5a} include cyclobutyl, cyclopentyl, bicyclo[1.1.1]pentanyl, phenyl, dihydrobenzofuranyl and pyrrolyl, any of which groups may be optionally substituted by one or more substituents.

Typical examples of optional substituents on R^{5a} include C₁₋₆ alkyl, halogen, cyano, trifluoromethyl, trifluoroethyl, phenyl, hydroxy, C₁₋₆ alkoxy, C₁₋₆ alkylthio, C₁₋₆ alkylsulfinyl, C₁₋₆ alkylsulfonyl, C₂₋₆ alkylcarbonyl, amino, C₁₋₆ alkylamino and di(C₁₋₆)alkylamino.

Suitable examples of optional substituents on R^{5a} include C₁₋₆ alkyl and halogen.

Typical examples of particular substituents on R^{5a} include methyl, fluoro, chloro, bromo, cyano, trifluoromethyl, trifluoroethyl, phenyl, hydroxy, methoxy, methylthio, methylsulfinyl, methylsulfonyl, acetyl, amino, methylamino and dimethylamino.

Suitable examples of particular substituents on R^{5a} include methyl and chloro.

Suitable values of R^{5a} include cyclobutyl, cyclopentyl, bicyclo[1.1.1]pentanyl, phenyl, chlorophenyl, dihydrobenzofuranyl and methylpyrrolyl.

Suitably, R^{5b} represents hydrogen, methyl or ethyl.

In a first embodiment, R^{5b} represents hydrogen. In a second embodiment, R^{5b} represents C₁₋₆ alkyl, especially methyl or ethyl.

Alternatively, R^{5a} and R^{5b}, when taken together with the carbon atom to which they are both attached, may represent C₃₋₇ cycloalkyl, C₄₋₉ bicycloalkyl or C₃₋₇ heterocycloalkyl, any of which groups may be unsubstituted, or substituted by one or more substituents, typically by one or two substituents.

In a first embodiment, R^{5a} and R^{5b}, when taken together with the carbon atom to which they are both attached, may suitably represent optionally substituted C₃₋₇ cycloalkyl. Typical examples include cyclobutyl, benzocyclobutenyl, cyclopentyl, indanyl, cyclohexyl, tetrahydronaphthalenyl, cycloheptanyl, benzocycloheptenyl, cyclooctanyl and cyclononanyl, any of which groups may be optionally substituted by one or more substituents. A particular example is benzocyclobutenyl, which group may be optionally substituted by one or more substituents.

In a second embodiment, R^{5a} and R^{5b}, when taken together with the carbon atom to which they are both attached, may suitably represent optionally substituted C₄₋₉ bicycloalkyl. Examples include bicyclo[3.1.0]hexanyl, bicyclo[2.2.1]heptanyl and bicyclo[3.2.1]octanyl, any of which groups may be optionally substituted by one or more substituents.

In a third embodiment, R^{5a} and R^{5b}, when taken together with the carbon atom to which they are both attached, may suitably represent optionally substituted C₃₋₇ heterocycloalkyl. Examples include tetrahydropyranyl and piperidinyl, either of which groups may be optionally substituted by one or more substituents.

Typical examples of optional substituents on such groups include C₁₋₆ alkyl, halogen, cyano, trifluoromethyl, trifluoroethyl, phenyl, hydroxy, C₁₋₆ alkoxy, C₁₋₆ alkylthio, C₁₋₆ alkylsulfinyl, C₁₋₆ alkylsulfonyl, C₂₋₆ alkylcarbonyl, amino, C₁₋₆ alkylamino and di(C₁₋₆)alkylamino.

Suitable examples of optional substituents on such groups include C₁₋₆ alkyl, halogen, trifluoromethyl, trifluoroethyl, phenyl and C₁₋₆ alkoxy, especially halogen.

Typical examples of particular substituents on such groups include methyl, fluoro, chloro, bromo, cyano, trifluoromethyl, trifluoroethyl, phenyl, hydroxy, methoxy, methylthio, methylsulfinyl, methylsulfonyl, acetyl, amino, methylamino and dimethylamino.

Suitable examples of particular substituents on such groups include methyl, chloro, trifluoromethyl, trifluoroethyl, phenyl and methoxy, especially chloro.

Typical values of R^{5a} and R^{5b}, when taken together with the carbon atom to which they are both attached, include methylcyclobutyl, dimethylcyclobutyl, phenylcyclobutyl, benzocyclobutenyl, methylbenzocyclobutenyl, chlorobenzocyclobutenyl, methoxy-benzocyclobutenyl, cyclopentyl, methylcyclopentyl, indanyl, chloroindanyl, cyclohexyl, methylcyclohexyl, dimethylcyclohexyl, trifluoromethylcyclohexyl, tetrahydronaphthalenyl, cycloheptanyl, benzocycloheptenyl, cyclooctanyl, cyclononanyl, bicyclo[3.1.0]hexanyl, bicyclo[2.2.1]heptanyl, bicyclo[3.2.1]octanyl, tetramethyltetrahydropyranyl and trifluoroethylpiperidinyl.

Suitable values of R^{5a} and R^{5b}, when taken together with the carbon atom to which they are both attached, include chlorobenzocyclobutenyl.

Generally, R⁶ represents —NR^{6a}R^{6b} or —OR^{6c}; or R⁶ represents C₁₋₉ alkyl, aryl, C₃₋₇ heterocycloalkyl, heteroaryl, heteroaryl(C₁₋₆)alkyl or spiro[(C₃₋₇)heterocycloalkyl]-[heteroaryl], any of which groups may be optionally substituted by one or more substituents.

Typically, R⁶ represents —NR^{6a}R^{6b}; or R⁶ represents aryl or heteroaryl, either of which groups may be optionally substituted by one or more substituents.

Appositely, R⁶ represents aryl or heteroaryl, either of which groups may be optionally substituted by one or more substituents.

Suitably, R⁶ represents heteroaryl, which group may be optionally substituted by one or more substituents.

In a first embodiment, R⁶ represents optionally substituted C₁₋₆ alkyl. In a second embodiment, R⁶ represents optionally substituted C₃₋₉ cycloalkyl. In a third embodiment, R⁶ represents optionally substituted C₃₋₉ cycloalkyl(C₁₋₆)alkyl. In a fourth embodiment, R⁶ represents optionally substituted aryl. In a fifth embodiment, R⁶ represents optionally substituted aryl(C₁₋₆)alkyl. In a sixth embodiment, R⁶ represents optionally substituted C₃₋₇ heterocycloalkyl. In a seventh embodiment, R⁶ represents optionally substituted C₃₋₇ heterocycloalkyl(C₁₋₆)alkyl. In an eighth embodiment, R⁶ represents optionally substituted heteroaryl. In a ninth embodiment, R⁶ represents optionally substituted heteroaryl(C₁₋₆)alkyl. In a tenth embodiment, R⁶ represents optionally substituted spiro[(C₃₋₇)heterocycloalkyl][heteroaryl]. In an eleventh embodiment, R⁶ represents —NR^{6a}R^{6b}. In a twelfth embodiment, R⁶ represents —OR^{6c}.

Typical values of R⁶ include —NR^{6a}R^{6b} and —OR^{6c}; and methyl, tert-butyl, heptanyl, phenyl, pyrrolidinyl, indolinyl, piperidinyl, morpholinyl, thiomorpholinyl, piperazinyl, pyrrolyl, pyrazolyl, pyrazolo[1,5-a]pyridinyl, 4,5,6,7-tetrahydropyrazolyl, oxazolyl, isoxazolyl, thiazolyl, isothiazolyl, imidazolyl, oxadiazolyl, thiadiazolyl, triazolyl, tetrazolyl, pyridinyl, pyridazinyl, pyrimidinyl, pyridinylmethyl or spiro[tetrahydrofuran]-[indole], any of which groups may be optionally substituted by one or more substituents.

Selected values of R⁶ include —NR^{6a}R^{6b}; and phenyl, pyrazolyl, isoxazolyl or oxadiazolyl, any of which groups may be optionally substituted by one or more substituents.

Suitable values of R⁶ include —NR^{6a}R^{6b}; and phenyl, pyrazolyl or isoxazolyl, any of which groups may be optionally substituted by one or more substituents.

Apposite values of R⁶ include pyrazolyl and isoxazolyl, either of which groups may be optionally substituted by one or more substituents.

Typical examples of optional substituents on R⁶ include one, two or three substituents independently selected from halogen, cyano, nitro, C₁₋₆ alkyl, difluoromethyl, trifluoromethyl, difluoroethyl, trifluoroethyl, trifluoropropyl, cyclopropyl, cyclobutyl, cyclopropylmethyl, phenyl, fluorophenyl, hydroxy, hydroxy(C₁₋₆)alkyl, oxo, C₁₋₆ alkoxy, C₁₋₆ alkoxy(C₁₋₆)alkyl, difluoromethoxy, trifluoromethoxy, C₁₋₆ alkylthio, C₁₋₆ alkylsulfinyl, C₁₋₆ alkylsulfonyl, (C₁₋₆)alkylsulfonyl(C₁₋₆)alkyl, C₁₋₆ alkylsulfonyloxy, amino, amino(C₁₋₆)alkyl, C₁₋₆ alkylamino, di(C₁₋₆)alkylamino, di(C₁₋₆)alkylamino(C₁₋₆)alkyl, pyrrolidinyl, dioxoisothiazolidinyl, tetrahydropyranyl, morpholinyl, piperazinyl, C₂₋₆ alkylcarbonylamino, C₂₋₆ alkylcarbonylamino(C₁₋₆)alkyl, C₂₋₆ alkoxycarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl, di(C₁₋₆)alkylaminosulfonyl and di(C₁₋₆)alkylsulfoximinyl.

Suitable examples of optional substituents on R⁶ include one, two or three substituents independently selected from C₁₋₆ alkyl, (C₁₋₆)alkylsulfonyl(C₁₋₆)alkyl, C₁₋₆ alkylsulfonylamino and di(C₁₋₆)alkylsulfoximinyl.

Typical examples of specific substituents on R⁶ include one, two or three substituents independently selected from fluoro, chloro, bromo, cyano, nitro, methyl, ethyl, n-propyl, isopropyl, 2-methylpropyl, butan-2-yl, tert-butyl, difluoromethyl, trifluoromethyl, difluoroethyl, trifluoroethyl, trifluoropropyl, cyclopropyl, cyclobutyl, cyclopropylmethyl, phenyl, fluorophenyl, hydroxy, hydroxymethyl, hydroxyethyl, oxo, methoxy, tert-butoxy, methoxymethyl, methoxyethyl, difluoromethoxy, trifluoromethoxy, methylthio, methylsulfinyl, methylsulfonyl, methylsulfonylmethyl, methylsulfonyloxy, amino, aminomethyl, aminoethyl, aminoisopropyl, methylamino, tert-butylamino, dimethylamino, dimethylaminoethyl, pyrrolidinyl, dioxoisothiazolidinyl, tetrahydropyranyl, morpholinyl, piperazinyl, acetylamino, acetylaminoethyl, methoxycarbonylamino, methylsulfonylamino, formyl, acetyl, carboxy, methoxycarbonyl, ethoxycarbonyl, tert-butoxycarbonyl, aminocarbonyl, methylaminocarbonyl, dimethylaminocarbonyl, aminosulfonyl, methylaminosulfonyl, dimethylaminosulfonyl and dimethylsulfoximinyl.

Suitable examples of specific substituents on R⁶ include one, two or three substituents independently selected from methyl, ethyl, methylsulfonylmethyl, methylsulfonylamino and dimethylsulfoximinyl.

Illustrative values of R⁶ include —NR^{6a}R^{6b}, —OR^{6c}, methyl, tert-butyl, hydroxyheptanyl, phenyl, fluorophenyl, methylsulfonylphenyl, methylsulfonylmethylphenyl, dioxoisothiazolidinylphenyl, methylsulfonylaminophenyl, dimethylsulfoximinylphenyl, pyrrolidinyl, methylpyrrolidinyl, indolinyl, piperidinyl, morpholinyl, dioxo-thiomorpholinyl, methylpiperazinyl, methylpyrrolyl, methylpyrazolyl, dimethylpyrazolyl, ethylpyrazolyl, (ethyl)(fluoro)pyrazolyl, (ethyl)(methyl)pyrazolyl, n-propylpyrazolyl, isopropylpyrazolyl, 2-methylpropylpyrazolyl, butan-2-ylpyrazolyl, difluoromethyl-pyrazolyl, (difluoromethyl)(methyl)pyrazolyl, difluoroethylpyrazolyl, trifluoroethyl-pyrazolyl, trifluoropropylpyrazolyl, cyclopropylpyrazolyl, cyclobutylpyrazolyl, cyclopropylmethylpyrazolyl, hydroxyethylpyrazolyl, methoxyethylpyrazolyl, dimethyl-aminoethylpyrazolyl, tetrahydropyranylpyrazolyl, (methyl)(tetrahydropyranyl)pyrazolyl, pyrazolo[1,5-a]pyridinyl, methyl-4,5,6,7-tetrahydropyrazolyl, oxazolyl, methyloxazolyl, ethyloxazolyl, isoxazolyl, methylisoxazolyl, dimethylisoxazolyl, ethylisoxazolyl, isopropylisoxazolyl, tert-butylisoxazolyl, trifluoromethylisoxazolyl, cyclopropyl-isoxazolyl, cyclobutylisoxazolyl, methoxymethylisoxazolyl, aminomethylisoxazolyl, aminoisopropylisoxazolyl, thiazolyl, methylthiazolyl, dimethylthiazolyl, isothiazolyl, methylisothiazolyl, methylimidazolyl, methyloxadiazolyl, ethyloxadiazolyl, methyl-thiadiazolyl, methyltriazolyl, dimethyltriazolyl, ethyltriazolyl, methyltetrazolyl, pyridinyl, methylpyridinyl, pyridazinyl, pyrimidinyl, methylpyrimidinyl, pyridinylmethyl, amino-pyridinylmethyl and spiro[tetrahydrofuran][oxoindole].

Selected values of R⁶ include —NR^{6a}R^{6b}, methylsulfonylmethylphenyl, methylsulfonylaminophenyl, dimethylsulfoximinylphenyl, ethylpyrazolyl, methylisoxazolyl ethylisoxazolyl and ethyloxadiazolyl.

Representative values of R⁶ include —NR^{6a}R^{6b} methylsulfonylmethylphenyl, methylsulfonylaminophenyl, dimethylsulfoximinylphenyl, ethylpyrazolyl, methylisoxazolyl and ethylisoxazolyl.

Apposite values of R⁶ include methylpyrazolyl, ethylpyrazolyl, methylisoxazolyl and ethylisoxazolyl.

Typically, R^{6a} represents C₁₋₆ alkyl, C₃₋₇ cycloalkyl, aryl(C₁₋₆)alkyl, C₃₋₇ heterocycloalkyl or spiro[(C₃₋₇)heterocycloalkyl][heteroaryl], any of which groups may be optionally substituted by one or more substituents.

In a first embodiment, R^{6a} represents hydrogen. In a second embodiment, R^{6a} represents optionally substituted C₁₋₆ alkyl. In a first aspect of that embodiment, R^{6a} represents unsubstituted C₁₋₆ alkyl, especially methyl. In a second aspect of that embodiment, R^{6a} represents monosubstituted, disubstituted or trisubstituted C₁₋₆ alkyl. In a third embodiment, R^{6a} represents optionally substituted C₃₋₇ cycloalkyl. In a fourth embodiment, R^{6a} represents optionally substituted C₃₋₇ cycloalkyl(C₁₋₆)alkyl. In a fifth embodiment, R^{6a} represents optionally substituted aryl. In a sixth embodiment, R^{6a} represents optionally substituted aryl(C₁₋₆)alkyl. In a seventh embodiment, R^{6a} represents optionally substituted C₃₋₇ heterocycloalkyl. In an eighth embodiment, R^{6a} represents optionally substituted C₃₋₇ heterocycloalkyl(C₁₋₆)alkyl. In a ninth embodiment, R^{6a} represents optionally substituted heteroaryl. In a tenth embodiment, R^{6a} represents optionally substituted heteroaryl(C₁₋₆)alkyl. In an eleventh embodiment, R^{6a} represents optionally substituted spiro[(C₃₋₇)heterocycloalkyl][heteroaryl].

Typical values of R^{6a} include methyl, ethyl, n-propyl, isopropyl, 2,2-dimethylpropyl, cyclohexyl, benzyl, tetrahydrofuranyl, tetrahydropyranyl, tetrahydrothiopyranyl and spiro[tetrahydrofuran][indole], any of which groups may be optionally substituted by one or more substituents.

Suitable values of R^{6a} include tetrahydropyranyl, which group may be optionally substituted by one or more substituents.

Typical examples of optional substituents on R^{6a} include one, two or three substituents independently selected from halogen, cyano, nitro, C₁₋₆ alkyl, trifluoromethyl, phenyl, fluorophenyl, hydroxy, hydroxy(C₁₋₆)alkyl, oxo, C₁₋₆ alkoxy, difluoromethoxy, trifluoromethoxy, C₁₋₆ alkylthio, C₁₋₆ alkylsulfinyl, C₁₋₆ alkylsulfonyl, amino, amino(C₁₋₆)alkyl, C₁₋₆ alkylamino, di(C₁₋₆)alkylamino, pyrrolidinyl, morpholinyl, piperazinyl, C₂₋₆ alkylcarbonylamino, C₂₋₆ alkylcarbonylamino(C₁₋₆)alkyl, C₂₋₆ alkoxycarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl and di(C₁₋₆)alkylaminosulfonyl.

Selected examples of optional substituents on R^{6a} include one, two or three substituents independently selected from trifluoromethyl, oxo and C₁₋₆ alkoxy.

Typical examples of specific substituents on R^{6a} include one, two or three substituents independently selected from fluoro, chloro, bromo, cyano, nitro, methyl, ethyl, isopropyl, tert-butyl, trifluoromethyl, phenyl, fluorophenyl, hydroxy, hydroxymethyl, oxo, methoxy, tert-butoxy, difluoromethoxy, trifluoromethoxy, methylthio, methylsulfinyl, methylsulfonyl, amino, aminomethyl, aminoethyl, methylamino, tert-butylamino, dimethylamino, pyrrolidinyl, morpholinyl, piperazinyl, acetylamino, acetylaminoethyl, methoxycarbonylamino, methylsulfonylamino, formyl, acetyl, carboxy, methoxycarbonyl, ethoxycarbonyl, tert-butoxycarbonyl, aminocarbonyl, methylaminocarbonyl, dimethylaminocarbonyl, aminosulfonyl, methylaminosulfonyl and dimethylaminosulfonyl.

Selected examples of specific substituents on R^{6a} include one, two or three substituents independently selected from trifluoromethyl, oxo and methoxy.

Selected values of R^{6a} include methyl, ethyl, trifluoroethyl, methoxyethyl, n-propyl, isopropyl, 2,2-dimethylpropyl, cyclohexyl, benzyl, tetrahydrofuranyl, tetrahydropyranyl, oxotetrahydrothiopyranyl and spiro[tetrahydrofuran][oxoindole].

A particular value of R^{6a} is tetrahydropyranyl.

Suitably, R^{6b} represents hydrogen, methyl, ethyl, n-propyl or isopropyl.

Typically, R^{6b} represents hydrogen or methyl.

In a first embodiment, R^{6b} represents hydrogen. In a second embodiment, R^{6b} represents C₁₋₆ alkyl. In a particular aspect of that embodiment, R^{6b} represents methyl, ethyl, n-propyl or isopropyl, especially methyl.

Typically, R^{6c} represents C₁₋₆ alkyl, C₃₋₇ cycloalkyl, C₃₋₇ cycloalkyl(C₁₋₆)alkyl, C₃₋₇ heterocycloalkyl, C₃₋₇ heterocycloalkyl(C₁₋₆)alkyl or heteroaryl(C₁₋₆)alkyl, any of which groups may be optionally substituted by one or more substituents.

In a first embodiment, R^{6c} represents optionally substituted C₁₋₆ alkyl. In a second embodiment, R^{6c} represents optionally substituted C₃₋₇ cycloalkyl. In a third embodiment, R^{6c} represents optionally substituted C₃₋₇ cycloalkyl(C₁₋₆)alkyl. In a fourth embodiment, R^{6c} represents optionally substituted aryl. In a fifth embodiment, R^{6c} represents optionally substituted aryl(C₁₋₆)alkyl. In a sixth embodiment, R^{6c} represents optionally substituted C₃₋₇ heterocycloalkyl. In a seventh embodiment, R^{6c} represents optionally substituted C₃₋₇ heterocycloalkyl(C₁₋₆)alkyl. In an eighth embodiment, R^{6c} represents optionally substituted heteroaryl. In a ninth embodiment, R^{6c} represents optionally substituted heteroaryl(C₁₋₆)alkyl.

Typical values of R^{6c} include methyl, ethyl, isopropyl, 2-methylpropyl, tert-butyl, 2,2-dimethylpropyl, cyclobutyl, cyclopentyl, cyclohexyl, cyclopropylmethyl, cyclohexylmethyl, oxetanyl, azetidinyl, tetrahydrofuranyl, tetrahydropyranyl, tetrahydropyranylmethyl, pyrazolylmethyl, oxazolylmethyl, isoxazolylmethyl, imidazolylmethyl and pyrazinylmethyl, any of which groups may be optionally substituted by one or more substituents.

Typical examples of optional substituents on R^{6c} include one, two or three substituents independently selected from halogen, cyano, nitro, C₁₋₆ alkyl, trifluoromethyl, phenyl, fluorophenyl, hydroxy, hydroxy(C₁₋₆)alkyl, oxo, C₁₋₆ alkoxy, difluoromethoxy, trifluoromethoxy, C₁₋₆ alkylthio, C₁₋₆ alkylsulfinyl, C₁₋₆ alkylsulfonyl, amino, amino(C₁₋₆)alkyl, C₁₋₆ alkylamino, di(C₁₋₆)alkylamino, pyrrolidinyl, morpholinyl, piperazinyl, C₂₋₆ alkylcarbonylamino, C₂₋₆ alkylcarbonylamino(C₁₋₆)alkyl, C₂₋₆ alkoxycarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl and di(C₁₋₆)alkylaminosulfonyl.

Suitable examples of optional substituents on R^{6c} include one, two or three substituents independently selected from C₁₋₆ alkyl, trifluoromethyl, C₁₋₆ alkoxy and C₂₋₆ alkoxycarbonyl.

Typical examples of specific substituents on R^{6c} include one, two or three substituents independently selected from fluoro, chloro, bromo, cyano, nitro, methyl, ethyl, isopropyl, tert-butyl, trifluoromethyl, phenyl, fluorophenyl, hydroxy, hydroxymethyl, oxo, methoxy, tert-butoxy, difluoromethoxy, trifluoromethoxy, methylthio, methylsulfinyl, methylsulfonyl, amino, aminomethyl, aminoethyl, methylamino, tert-butylamino, dimethylamino, pyrrolidinyl, morpholinyl, piperazinyl, acetylamino, acetylaminoethyl, methoxycarbonylamino, methylsulfonylamino, formyl, acetyl, carboxy, methoxycarbonyl, ethoxycarbonyl, tert-butoxycarbonyl, aminocarbonyl, methylaminocarbonyl, dimethylaminocarbonyl, aminosulfonyl, methylaminosulfonyl and dimethylaminosulfonyl.

Suitable examples of specific substituents on R^{6c} include one, two or three substituents independently selected from methyl, trifluoromethyl, methoxy and tert-butoxycarbonyl.

Typical values of R^{6c} include methyl, trifluoroethyl, methoxyethyl, isopropyl, 2-methylpropyl, tert-butyl, 2,2-dimethylpropyl, cyclobutyl, cyclopentyl, cyclohexyl, cyclopropylmethyl, cyclohexylmethyl, oxetanyl, methyloxetanyl, azetidinyl, tert-butoxycarbonylazetidinyl, tetrahydrofuranyl, tetrahydropyranyl, tetrahydropyranylmethyl, methylpyrazolylmethyl, oxazolylmethyl, isoxazolylmethyl, methylimidazolylmethyl and pyrazinylmethyl.

In a first embodiment, R⁷ represents aryl, which group may be optionally substituted by one or more substituents. In a second embodiment, R⁷ represents heteroaryl, which group may be optionally substituted by one or more substituents. In a third embodiment, R⁷ represents spiro[(C₃₋₇)heterocycloalkyl][heteroaryl], which group may be optionally substituted by one or more substituents.

Typical values of R⁷ include phenyl, pyrazolo[1,5-a]pyrazinyl, benzoxazolyl, benzothiazolyl, benzimidazolyl, imidazo[1,2-b]pyridazinyl, purinyl, pyridinyl, pyridazinyl, cinnolinyl, pyrimidinyl, pyrazinyl and spiro[tetrahydropyranyl][indole], any of which groups may be optionally substituted by one or more substituents.

Typical examples of optional substituents on R⁷ include one, two or three substituents independently selected from halogen, cyano, nitro, C₁₋₆ alkyl, difluoromethyl, trifluoromethyl, phenyl, fluorophenyl, hydroxy, hydroxy(C₁₋₆)alkyl, oxo, C₁₋₆ alkoxy, difluoromethoxy, trifluoromethoxy, C₁₋₆ alkylthio, C₁₋₆ alkylsulfinyl, C₁₋₆ alkylsulfonyl, amino, amino(C₁₋₆)alkyl, C₁₋₆ alkylamino, di(C₁₋₆)alkylamino, pyrrolidinyl, morpholinyl, piperazinyl, C₂₋₆ alkylcarbonylamino, C₂₋₆ alkylcarbonylamino(C₁₋₆)alkyl, C₂₋₆ alkoxycarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl and di(C₁₋₆)alkylaminosulfonyl.

Suitable examples of optional substituents on R⁷ include one, two or three substituents independently selected from halogen, cyano, C₁₋₆ alkyl, difluoromethyl, trifluoromethyl, oxo, C₁₋₆ alkoxy, difluoromethoxy and di(C₁₋₆)alkylamino.

Typical examples of specific substituents on R⁷ include one, two or three substituents independently selected from fluoro, chloro, bromo, cyano, nitro, methyl, ethyl, isopropyl, tert-butyl, difluoromethyl, trifluoromethyl, phenyl, fluorophenyl, hydroxy, hydroxymethyl, oxo, methoxy, isopropoxy, tert-butoxy, difluoromethoxy, trifluoromethoxy, methylthio, methylsulfinyl, methylsulfonyl, amino, aminomethyl, aminoethyl, methylamino, tert-butylamino, dimethylamino, pyrrolidinyl, morpholinyl, piperazinyl, acetylamino, acetylaminoethyl, methoxycarbonylamino, methylsulfonylamino, formyl, acetyl, carboxy, methoxycarbonyl, ethoxycarbonyl, tert-butoxycarbonyl, aminocarbonyl, methylaminocarbonyl, dimethylaminocarbonyl, aminosulfonyl, methylaminosulfonyl and dimethylaminosulfonyl.

Suitable examples of specific substituents on R⁷ include one, two or three substituents independently selected from fluoro, chloro, cyano, methyl, ethyl, isopropyl, difluoromethyl, trifluoromethyl, oxo, methoxy, isopropoxy, difluoromethoxy and dimethylamino.

Selected values of R⁷ include phenyl, pyrazolo[1,5-a]pyrazinyl, benzoxazolyl, fluorobenzoxazolyl, methylbenzoxazolyl, benzothiazolyl, benzimidazolyl, fluoro-benzimidazolyl, imidazo[1,2-b]pyridazinyl, purinyl, pyridinyl, cyanopyridinyl, methylpyridinyl, methoxypyridinyl, pyridazinyl, chloropyridazinyl, cyanopyridazinyl, methylpyridazinyl, ethylpyridazinyl, isopropylpyridazinyl, difluoromethylpyridazinyl, trifluoro-methylpyridazinyl, methoxypyridazinyl, isopropoxypyridazinyl, difluoromethoxy-pyridazinyl, dimethylaminopyridazinyl, cinnolinyl, pyrimidinyl, pyrazinyl, methyl-pyrazinyl and spiro[tetrahydropyranyl][oxoindole].

A particular sub-class of the compounds of formula (IA) above is represented by the compounds of formula (IIA), and pharmaceutically acceptable salts thereof:

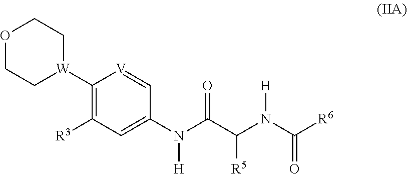

wherein

V represents N or C—R²;

W represents N or C—R¹¹;

R² represents hydrogen, halogen, cyano, C₁₋₆ alkyl, fluoromethyl, difluoromethyl, trifluoromethyl, hydroxy, C₁₋₆ alkoxy, difluoromethoxy, trifluoromethoxy, C₁₋₆ alkylthio, C₁₋₆ alkylsulfinyl, C₁₋₆ alkylsulfonyl, amino, C₁₋₆ alkylamino, di(C₁₋₆)alkylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl or di(C₁₋₆)alkylaminosulfonyl;

R³ represents hydrogen, halogen, C₁₋₆ alkyl or C₁₋₆ alkoxy;

R¹¹ (represents hydrogen, C₁₋₆ alkyl, halogen, cyano, trifluoromethyl, hydroxy, hydroxy(C₁₋₆)alkyl, C₁₋₆ alkoxy, C₁₋₆ alkylthio, C₁₋₆ alkylsulfinyl, C₁₋₆ alkylsulfonyl, C₂₋₆ alkylcarbonyl, amino, C₁₋₆ alkylamino, di(C₁₋₆)alkylamino, aminocarbonyl, C₁₋₆ alkylaminocarbonyl, di(C₁₋₆)alkylaminocarbonyl or difluoroazetidinylcarbonyl; and

R⁵ and R⁶ are as defined above.

In a first embodiment, V is N. In a second embodiment, V is C—R².

In a first embodiment, W is N. In a second embodiment, W is C—R¹¹.

Typically, R² represents hydrogen, halogen, C₁₋₆ alkyl or C₁₋₆ alkoxy.

In a first embodiment, R² represents hydrogen. In a second embodiment, R² represents halogen. In a first aspect of that embodiment, R² represents fluoro. In a second aspect of that embodiment, R² represents chloro. In a third aspect of that embodiment, R² represents bromo. In a third embodiment, R² represents C₁₋₆ alkyl, especially methyl. In a fourth embodiment, R² represents C₁₋₆ alkoxy, especially methoxy.

Suitably, R² represents hydrogen, fluoro, chloro, bromo, methyl or methoxy.

Typically, R³ represents hydrogen or halogen.

In a first embodiment, R³ represents hydrogen. In a second embodiment, R³ represents halogen. In a first aspect of that embodiment, R³ represents fluoro. In a second aspect of that embodiment, R³ represents chloro.

Appositely, R³ represents hydrogen, fluoro or chloro.

Suitably, R³ represents hydrogen or fluoro.

Generally, R¹¹ represents hydrogen, C₁₋₆ alkyl, halogen, cyano, trifluoromethyl, hydroxy, hydroxy(C₁₋₆)alkyl, C₁₋₆ alkoxy, C₁₋₆ alkylthio, C₁₋₆ alkylsulfinyl, C₁₋₆ alkylsulfonyl, C₂₋₆ alkylcarbonyl, amino, C₁₋₆ alkylamino, di(C₁₋₆)alkylamino, aminocarbonyl, C₁₋₆ alkylaminocarbonyl or di(C₁₋₆)alkylaminocarbonyl.

Typically, R¹¹ represents hydrogen, cyano, hydroxy, hydroxy(C₁₋₆)alkyl, C₁₋₆ alkoxy, di(C₁₋₆)alkylaminocarbonyl or difluoroazetidinylcarbonyl.

Suitably, R¹¹ represents hydrogen, cyano, hydroxy, hydroxy(C₁₋₆)alkyl, C₁₋₆ alkoxy or di(C₁₋₆)alkylaminocarbonyl.

Typical values of R¹¹ include hydrogen, methyl, fluoro, chloro, bromo, cyano, trifluoromethyl, hydroxy, hydroxymethyl, methoxy, methylthio, methylsulfinyl, methylsulfonyl, acetyl, amino, methylamino, dimethylamino, aminocarbonyl, methylaminocarbonyl and dimethylaminocarbonyl. Additional values include difluoroazetidinylcarbonyl.

Selected values of R¹¹ include hydrogen, cyano, hydroxy, hydroxymethyl, methoxy, dimethylaminocarbonyl and difluoroazetidinylcarbonyl.

Suitable values of R¹¹ include hydrogen, cyano, hydroxy, hydroxymethyl, methoxy and dimethylaminocarbonyl.

In a first embodiment, R¹¹ is hydrogen. In a second embodiment, R¹¹ is other than hydrogen.

Specific novel compounds in accordance with the present invention include each of the compounds whose preparation is described in the accompanying Examples, and pharmaceutically acceptable salts and solvates thereof.

The compounds in accordance with the present invention are beneficial in the treatment and/or prevention of various human ailments, including inflammatory and autoimmune disorders.

The compounds according to the present invention are useful in the treatment and/or prophylaxis of a pathological disorder that is mediated by a pro-inflammatory IL-17 cytokine or is associated with an increased level of a pro-inflammatory IL-17 cytokine. Generally, the pathological condition is selected from the group consisting of infections (viral, bacterial, fungal and parasitic), endotoxic shock associated with infection, arthritis, rheumatoid arthritis, psoriatic arthritis, systemic onset juvenile idiopathic arthritis (JIA), systemic lupus erythematosus (SLE), asthma, chronic obstructive airways disease (COAD), chronic obstructive pulmonary disease (COPD), acute lung injury, pelvic inflammatory disease, Alzheimer's Disease, Crohn's disease, inflammatory bowel disease, irritable bowel syndrome, ulcerative colitis, Castleman's disease, ankylosing spondylitis and other spondyloarthropathies, dermatomyositis, myocarditis, uveitis, exophthalmos, autoimmune thyroiditis, Peyronie's Disease, coeliac disease, gall bladder disease, Pilonidal disease, peritonitis, psoriasis, atopic dermatitis, vasculitis, surgical adhesions, stroke, autoimmune diabetes, Type I Diabetes, lyme arthritis, meningoencephalitis, immune mediated inflammatory disorders of the central and peripheral nervous system such as multiple sclerosis and Guillain-Barr syndrome, other autoimmune disorders, pancreatitis, trauma (surgery), graft-versus-host disease, transplant rejection, fibrosing disorders including pulmonary fibrosis, liver fibrosis, renal fibrosis, scleroderma or systemic sclerosis, cancer (both solid tumours such as melanomas, hepatoblastomas, sarcomas, squamous cell carcinomas, transitional cell cancers, ovarian cancers and hematologic malignancies and in particular acute myelogenous leukaemia, chronic myelogenous leukemia, chronic lymphatic leukemia, gastric cancer and colon cancer), heart disease including ischaemic diseases such as myocardial infarction as well as atherosclerosis, intravascular coagulation, bone resorption, osteoporosis, periodontitis, hypochlorhydia and pain (particularly pain associated with inflammation).

WO 2009/089036 reveals that modulators of IL-17 activity may be administered to inhibit or reduce the severity of ocular inflammatory disorders, in particular ocular surface inflammatory disorders including Dry Eye Syndrome (DES). Consequently, the compounds in accordance with the present invention are useful in the treatment and/or prevention of an IL-17-mediated ocular inflammatory disorder, in particular an IL-17-mediated ocular surface inflammatory disorder including Dry Eye Syndrome. Ocular surface inflammatory disorders include Dry Eye Syndrome, penetrating keratoplasty, corneal transplantation, lamellar or partial thickness transplantation, selective endothelial transplantation, corneal neovascularization, keratoprosthesis surgery, corneal ocular surface inflammatory conditions, conjunctival scarring disorders, ocular autoimmune conditions, Pemphigoid syndrome, Stevens-Johnson syndrome, ocular allergy, severe allergic (atopic) eye disease, conjunctivitis and microbial keratitis. Particular categories of Dry Eye Syndrome include keratoconjunctivitis sicca (KCS), Sjögren syndrome, Sjögren syndrome-associated keratoconjunctivitis sicca, non-Sjögren syndrome-associated keratoconjunctivitis sicca, keratitis sicca, sicca syndrome, xerophthalmia, tear film disorder, decreased tear production, aqueous tear deficiency (ATD), meibomian gland dysfunction and evaporative loss.

Illustratively, the compounds of the present invention may be useful in the treatment and/or prophylaxis of a pathological disorder selected from the group consisting of arthritis, rheumatoid arthritis, psoriasis, psoriatic arthritis, systemic onset juvenile idiopathic arthritis (JIA), systemic lupus erythematosus (SLE), asthma, chronic obstructive airway disease, chronic obstructive pulmonary disease, atopic dermatitis, scleroderma, systemic sclerosis, lung fibrosis, inflammatory bowel diseases (including Crohn's disease and ulcerative colitis), ankylosing spondylitis and other spondyloarthropathies, cancer and pain (particularly pain associated with inflammation).

Suitably, the compounds of the present invention are useful in the treatment and/or prophylaxis of psoriasis, psoriatic arthritis or ankylosing spondylitis.

The present invention also provides a pharmaceutical composition which comprises a compound in accordance with the invention as described above, or a pharmaceutically acceptable salt thereof, in association with one or more pharmaceutically acceptable carriers.

Pharmaceutical compositions according to the invention may take a form suitable for oral, buccal, parenteral, nasal, topical, ophthalmic or rectal administration, or a form suitable for administration by inhalation or insufflation.

For oral administration, the pharmaceutical compositions may take the form of, for example, tablets, lozenges or capsules prepared by conventional means with pharmaceutically acceptable excipients such as binding agents (e.g. pregelatinised maize starch, polyvinylpyrrolidone or hydroxypropyl methyl cellulose); fillers (e.g. lactose, microcrystalline cellulose or calcium hydrogenphosphate); lubricants (e.g. magnesium stearate, talc or silica); disintegrants (e.g. potato starch or sodium glycollate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents, emulsifying agents, non-aqueous vehicles or preservatives. The preparations may also contain buffer salts, flavouring agents, colouring agents or sweetening agents, as appropriate.

Preparations for oral administration may be suitably formulated to give controlled release of the active compound.

For buccal administration, the compositions may take the form of tablets or lozenges formulated in conventional manner.

The compounds according to the present invention may be formulated for parenteral administration by injection, e.g. by bolus injection or infusion. Formulations for injection may be presented in unit dosage form, e.g. in glass ampoules or multi-dose containers, e.g. glass vials. The compositions for injection may take such forms as suspensions, solutions or emulsions in oily or aqueous vehicles, and may contain formulatory agents such as suspending, stabilising, preserving and/or dispersing agents. Alternatively, the active ingredient may be in powder form for constitution with a suitable vehicle, e.g. sterile pyrogen-free water, before use.

In addition to the formulations described above, the compounds according to the present invention may also be formulated as a depot preparation. Such long-acting formulations may be administered by implantation or by intramuscular injection.

For nasal administration or administration by inhalation, the compounds according to the present invention may be conveniently delivered in the form of an aerosol spray presentation for pressurised packs or a nebuliser, with the use of a suitable propellant, e.g. dichlorodifluoromethane, fluorotrichloromethane, dichlorotetrafluoroethane, carbon dioxide or other suitable gas or mixture of gases.

The compositions may, if desired, be presented in a pack or dispenser device which may contain one or more unit dosage forms containing the active ingredient. The pack or dispensing device may be accompanied by instructions for administration.

For topical administration the compounds according to the present invention may be conveniently formulated in a suitable ointment containing the active component suspended or dissolved in one or more pharmaceutically acceptable carriers. Particular carriers include, for example, mineral oil, liquid petroleum, propylene glycol, polyoxyethylene, polyoxypropylene, emulsifying wax and water. Alternatively, the compounds according to the present invention may be formulated in a suitable lotion containing the active component suspended or dissolved in one or more pharmaceutically acceptable carriers. Particular carriers include, for example, mineral oil, sorbitan monostearate, polysorbate 60, cetyl esters wax, cetearyl alcohol, benzyl alcohol, 2-octyldodecanol and water.

For ophthalmic administration the compounds according to the present invention may be conveniently formulated as micronized suspensions in isotonic, pH-adjusted sterile saline, either with or without a preservative such as a bactericidal or fungicidal agent, for example phenylmercuric nitrate, benzylalkonium chloride or chlorhexidine acetate. Alternatively, for ophthalmic administration the compounds according to the present invention may be formulated in an ointment such as petrolatum.

For rectal administration the compounds according to the present invention may be conveniently formulated as suppositories. These can be prepared by mixing the active component with a suitable non-irritating excipient which is solid at room temperature but liquid at rectal temperature and so will melt in the rectum to release the active component. Such materials include, for example, cocoa butter, beeswax and polyethylene glycols.

The quantity of a compound according to the present invention required for the prophylaxis or treatment of a particular condition will vary depending on the compound chosen and the condition of the patient to be treated. In general, however, daily dosages may range from around 10 ng/kg to 1000 mg/kg, typically from 100 ng/kg to 100 mg/kg, e.g. around 0.01 mg/kg to 40 mg/kg body weight, for oral or buccal administration, from around 10 ng/kg to 50 mg/kg body weight for parenteral administration, and from around 0.05 mg to around 1000 mg, e.g. from around 0.5 mg to around 1000 mg, for nasal administration or administration by inhalation or insufflation.

If desired, a compound in accordance with the present invention may be co-administered with another pharmaceutically active agent, e.g. an anti-inflammatory molecule.

The compounds of formula (I) above wherein R¹ represents —COR^{a} may be prepared by a process which comprises reacting a carboxylic acid of formula R^{a}CO₂H, or a salt thereof, e.g. a lithium salt thereof, with a compound of formula (III):

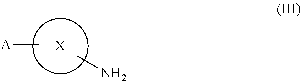

wherein X, A and R^{a} are as defined above.

The reaction is conveniently accomplished in the presence of a coupling agent. Suitable coupling agents may comprise the following:
2-(7-aza-1H-benzotriazol-1-yl)-1,1,3,3-tetramethyluronium hexafluorophosphate (HATU);
2,4,6-tripropyl-1,3,5,2,4,6-trioxatriphosphorinane 2,4,6-trioxide (propylphosphonic anhydride); or
a mixture of N-(3-dimethylaminopropyl)-N′-ethylcarbodiimide hydrochloride and 1-hydroxybenzotriazole.

The reaction is generally carried out in the presence of a base. Suitable bases include organic amines, e.g. a trialkylamine such as N,N-diisopropylethylamine or triethylamine. The reaction is conveniently performed at ambient or elevated temperature in a suitable solvent, e.g. a cyclic ether such as tetrahydrofuran, or a dipolar aprotic solvent such as N,N-dimethylformamide, or a chlorinated solvent such as dichloromethane.

Alternatively, the reaction may be accomplished in a two-step procedure which comprises: (i) treating a carboxylic acid of formula R^{a}CO₂H, or a salt thereof, e.g. a lithium salt thereof, with N-(3-dimethylaminopropyl)-N′-ethylcarbodiimide hydrochloride; and (ii) reacting the resulting material with compound (III) in the presence of acetic acid. Step (i) is conveniently effected at ambient temperature in a suitable solvent, e.g. a chlorinated solvent such as dichloromethane. Step (ii) is conveniently carried out at an elevated temperature in a suitable solvent, e.g. a cyclic ether such as tetrahydrofuran.

Where R^{a} represents —CH(R⁵)N(H)C(O)R⁶, the intermediates of formula R^{a}CO₂H may be prepared by a two-step procedure which comprises: (i) reacting a carboxylic acid of formula R⁶—CO₂H with a compound of formula (IV):

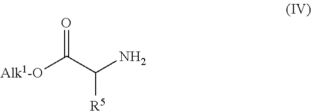

wherein Alk¹ represents C₁₋₄ alkyl, e.g. methyl, and R⁵ and R⁶ are as defined above; under conditions analogous to those described above for the reaction between compound (III) and a carboxylic acid of formula R^{a}CO₂H; and (ii) saponification of the resulting material by treatment with a base.

The saponification reaction in step (ii) will generally be effected by treatment with a base. Suitable bases include inorganic hydroxides, e.g. an alkali metal hydroxide such as lithium hydroxide. Where lithium hydroxide is employed in step (ii) of the above procedure, the product may be the lithium salt of the carboxylic acid of formula R^{a}CO₂H.

Step (ii) is conveniently effected at ambient temperature in water and a suitable organic solvent, e.g. a cyclic ether such as tetrahydrofuran, optionally in admixture with a C₁₋₄ alkanol such as methanol.

In another procedure, the compounds of formula (I) above wherein R¹ represents —SO₂R^{b} may be prepared by a process which comprises reacting a compound of formula R^{b}SO₂Cl with a compound of formula (III) as defined above.

The reaction is conveniently accomplished at ambient temperature in the presence of a base, e.g. an organic base such as triethylamine, in a suitable solvent, e.g. a chlorinated hydrocarbon solvent such as dichloromethane.

In another procedure, the compounds of formula (I) above wherein R¹ represents —COR^{a} may be prepared by a process which comprises reacting an amide of formula R^{a}CONH₂ with a compound of formula (V):

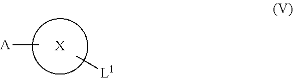

wherein X, A and R^{a} are as defined above, and L¹ represents a suitable leaving group; in the presence of a transition metal catalyst.

The leaving group L¹ is suitably a halogen atom, e.g. chloro or bromo.

The transition metal catalyst is suitably [(2-di-tert-butylphosphino-3,6-dimethoxy-2′,4′,6′-triisopropyl-1,1′-biphenyl)-2-(2′-amino-1,1′-biphenyl)]palladium(II) methanesulfonate (tBuBrettPhos Pd G3), in which case the reaction will generally be performed in the presence of 2-(di-tert-butylphosphino)-2′,4′,6′-triisopropyl-3,6-dimethoxy-1,1′-biphenyl (tBuBrettPhos). The reaction is conveniently carried out at an elevated temperature in the presence of a base, e.g. an inorganic base such as potassium carbonate, in a suitable solvent, e.g. a lower alkanol such as tert-butanol.

Alternatively, the transition metal catalyst may suitably be tris(dibenzylidene-acetone)dipalladium(0), in which case the reaction will generally be performed in the presence of 2-dicyclohexylphosphino-2′,4′,6′-triisopropylbiphenyl (XPhos) or 4,5-bis(diphenylphosphino)-9,9-dimethylxanthene (Xantphos). The reaction is conveniently carried out at an elevated temperature in the presence of a base, e.g. a carbonate salt such as potassium carbonate or cesium carbonate, in a suitable solvent, e.g. a cyclic ether such as 1,4-dioxane, or a C₁₋₆ alkanol such as tert-butanol.

In another procedure, the compounds of formula (I) above wherein R¹ is an aryl or heteroaryl moiety may be prepared by a process which comprises reacting a compound of formula R¹—NH₂ with a compound of formula (V) as defined above in the presence of a transition metal catalyst.

The transition metal catalyst is suitably tris(dibenzylideneacetone)dipalladium(0), in which case the reaction will generally be performed in the presence of 2-(di-tert-butyl)-phosphino-2′,4′,6′-triisopropylbiphenyl (tert-BuXPhos). The reaction is conveniently carried out at an elevated temperature in the presence of a base, e.g. a tert-butoxide salt such as sodium tert-butoxide, in a suitable solvent, e.g. a cyclic ether such as 1,4-dioxane.

The intermediates of formula (III) above may be prepared from the corresponding compound of formula (V) above by a two-step procedure which comprises: (i) reaction of compound (V) with tert-butyl carbamate in the presence of a transition metal catalyst; and (ii) removal of the tert-butoxycarbonyl (BOC) group from the material thereby obtained by treatment with an acid.

The transition metal catalyst of use in step (i) above is suitably palladium(II) acetate, in which case the reaction will generally be performed in the presence of 2-dicyclohexylphosphino-2′,4′,6′-triisopropylbiphenyl (XPhos). The reaction is conveniently carried out at an elevated temperature in the presence of a base, e.g. a carbonate salt such as potassium carbonate or cesium carbonate, in a suitable solvent, e.g. an aromatic hydrocarbon such as toluene.

Removal of the BOC group in step (ii) is conveniently effected by treatment with a mineral acid such as hydrochloric acid, or an organic acid such as trifluoroacetic acid.

In another procedure, the compounds of formula (IA) above may be prepared by a process which comprises reacting a compound of formula (III) as defined above with a compound of formula (VI):

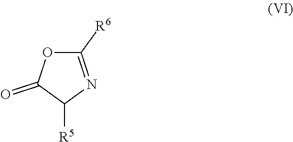

wherein R⁵ and R⁶ are as defined above.

The reaction between compounds (III) and (VI) will generally be performed in the presence of acetic acid. The reaction is conveniently carried out at an elevated temperature in a suitable solvent, e.g. a cyclic ether such as tetrahydrofuran.

Similarly, the compounds of formula (IF) above may be prepared by a process which comprises reacting a compound of formula (III) as defined above with a compound of formula (VII):

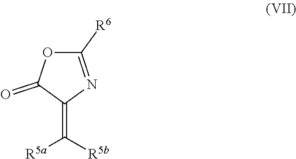

wherein R^{5a}, R^{5b} and R⁶ are as defined above; under conditions analogous to those described above for the reaction between compounds (III) and (VI).

Where the respective values of R⁵, R^{5a} and R^{5b} permit, an intermediate of formula (VI) may be obtained from the corresponding intermediate of formula (VII) by conventional catalytic hydrogenation.

The intermediates of formula (VII) above may be prepared by reacting a compound of formula R^{5a} C(O)R^{5b} with a compound of formula (VI) as defined above wherein R⁵ represents hydrogen.

The reaction is conveniently effected by treating the reagents with titanium tetrachloride; followed by treatment of the resulting material with pyridine.

In another procedure, the compounds of formula (IA) above may be prepared by a process which comprises reacting a carboxylic acid of formula R⁶—CO₂H with a compound of formula (VIII):

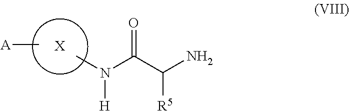

wherein X, A, R⁵ and R⁶ are as defined above; under conditions analogous to those described above for the reaction between compound (III) and a carboxylic acid of formula R^{a}CO₂H.

Similarly, the compounds of formula (IA) above wherein R⁶ represents —NR^{6a}R^{6b} may be prepared by a process which comprises reacting a carbamate derivative of formula L²-C(O)NR^{6a}R^{6b}, wherein L² represents a suitable leaving group, with a compound of formula (VIII) as defined above.

The leaving group L² is suitably a halogen atom, e.g. chloro; or L² is suitably phenoxy.

Where L² is a halogen atom, the reaction is conveniently carried out at ambient temperature in the presence of a base, e.g. a trialkylamine such as N,N-diisopropylethylamine or triethylamine, in a suitable solvent, e.g. a chlorinated solvent such as dichloromethane.

Where L² is phenoxy, the reaction is conveniently carried out at an elevated temperature in the presence of 4-(dimethylamino)pyridine, in a suitable solvent, e.g. a nitrile solvent such as acetonitrile.

Similarly, the compounds of formula (IA) above wherein R⁶ represents —OR^{6c} may be prepared by a process which comprises reacting a compound of formula L³-C(O)OR^{6c}, wherein L³ represents a suitable leaving group, with a compound of formula (VIII) as defined above.

The leaving group L³ is suitably a halogen atom, e.g. chloro.

The reaction is conveniently carried out at ambient temperature in the presence of a base, e.g. an organic amine such as triethylamine, typically in admixture with pyridine, in a suitable solvent, e.g. a cyclic ether such as tetrahydrofuran.

In another procedure, the compounds of formula (IB) above may be prepared by a process which comprises reacting a compound of formula (VIII) as defined above with a compound of formula L⁴-S(O)₂R⁶, wherein R⁶ is as defined above, and L⁴ represents a suitable leaving group.

The leaving group L⁴ is suitably a halogen atom, e.g. chloro.

The reaction is conveniently carried out at ambient temperature in the presence of a base, e.g. an organic amine such as N,N-diisopropylethylamine, in a suitable solvent, e.g. a chlorinated solvent such as dichloromethane.

In another procedure, the compounds of formula (IC) above may be prepared by a process which comprises reacting a compound of formula (VIII) as defined above with a compound of formula L⁵-R⁷, wherein R⁷ is as defined above, and L⁵ represents a suitable leaving group.

The leaving group L⁵ is suitably a halogen atom, e.g. chloro or bromo.

The reaction is conveniently carried out in the presence of a base. Suitable bases include organic amines, e.g. a trialkylamine such as N,N-diisopropylethylamine. The reaction is typically performed at an elevated temperature in a suitable solvent, e.g. a cyclic ether such as 1,4-dioxane.

Alternatively, the reaction may be performed in the presence of a transition metal catalyst. Suitable transition metal catalysts of use in this procedure include [(2-di-tert-butylphosphino-3,6-dimethoxy-2′,4′,6′-triisopropyl-1,1′-biphenyl)-2-(2′-amino-1,1′-biphenyl)]palladium(II) methanesulfonate (tBuBrettPhos Pd G3). The reaction is conveniently carried out at an elevated temperature in the presence of a base, e.g. an inorganic base such as potassium tert-butoxide, in a suitable solvent or solvent mixture. The solvent or solvents may suitably be selected from a cyclic ether such as 1,4-dioxane, and a sulfoxide solvent such as dimethyl sulfoxide.

The intermediates of formula (VIII) above may be prepared by reacting a compound of formula (III) as defined above with a compound of formula (IX), or a salt thereof, e.g. a lithium salt thereof:

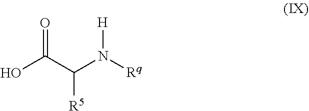

wherein R⁵ is as defined above, and R^{q} represents hydrogen or an N-protecting group;

under conditions analogous to those described above for the reaction between compound (III) and a carboxylic acid of formula R^{a}CO₂H; followed, as necessary, by removal of the N-protecting group R^{q}.

The N-protecting group R^{q} will suitably be tert-butoxycarbonyl (BOC).

Where the N-protecting group R^{q} is BOC, the subsequent removal thereof may conveniently be effected by treatment with an acid, e.g. a mineral acid such as hydrochloric acid, or an organic acid such as trifluoroacetic acid.

In another procedure, the compounds of formula (ID) above may be prepared by a process which comprises reacting a compound of formula R⁷—NH₂ with a compound of formula (X):

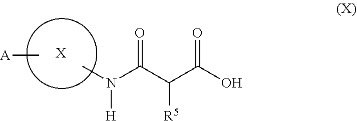

wherein X, A, R⁵ and R⁷ are as defined above; under conditions analogous to those described above for the reaction between compound (III) and a carboxylic acid of formula R^{a}CO₂H.

The intermediates of formula (X) above may be prepared by a two-step procedure which comprises: (i) reacting a compound of formula (III) as defined above with a compound of formula (XI), or a salt thereof, e.g. a lithium salt thereof:

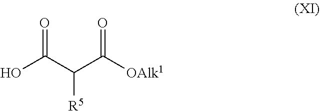

wherein R⁵ and Alk¹ are as defined above; under conditions analogous to those described above for the reaction between compound (III) and a carboxylic acid of formula R^{a}CO₂H; and (ii) saponification of the resulting material by treatment with a base.

The saponification reaction in step (ii) will generally be effected by treatment with a base. Suitable bases include inorganic hydroxides, e.g. an alkali metal hydroxide such as lithium hydroxide. Where lithium hydroxide is employed in step (ii) of the above procedure, the product may be the lithium salt of the carboxylic acid of formula (X).

Step (ii) is conveniently effected at ambient temperature in water and a suitable organic solvent, e.g. a C₁₋₄ alkanol such as ethanol.

The compounds of formula (IA) above may alternatively be prepared by a two-step procedure which comprises:

(i) reacting a compound of formula (XII):

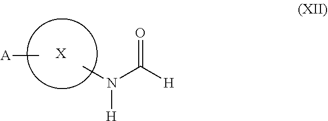

wherein X and A are as defined above; with phosphorus oxychloride; and

(ii) reacting the resulting material with a compound of formula R⁵—CHO and a compound of formula R⁶—CO₂H in the presence of ammonia.

Step (i) is conveniently carried out in the presence of a base. Suitable bases include organic amines, e.g. a trialkylamine such as triethylamine. The reaction is typically performed at ambient temperature in a suitable solvent, e.g. a chlorinated solvent such as dichloromethane.

Step (ii) is suitably effected at ambient temperature in a suitable solvent, e.g. a mixture of 2,2,2-trifluoroethanol and a lower alkanol such as methanol.

Where they are not commercially available, the starting materials of formula (IV), (V), (IX), (XI) and (XII) may be prepared by methods analogous to those described in the accompanying Examples, or by standard methods well known from the art.

It will be understood that any compound of formula (I) initially obtained from any of the above processes may, where appropriate, subsequently be elaborated into a further compound of formula (I) by techniques known from the art. By way of example, a compound of formula (I) comprising a N-BOC moiety (wherein BOC is an abbreviation for tert-butoxycarbonyl) may be converted into the corresponding compound comprising a N—H moiety by treatment with an acid, e.g. a mineral acid such as hydrochloric acid, or an organic acid such as trifluoroacetic acid.

A compound of formula (I) comprising an amino (—NH₂) moiety may be acylated, e.g. acetylated, by treatment with a suitable acyl halide, e.g. acetyl chloride, typically in the presence of a base, e.g. an organic base such as N,N-diisopropylethylamine.

A compound which contains an N—H moiety may be alkylated, e.g. methylated, by treatment with the appropriate alkyl halide, e.g. iodomethane, typically at ambient temperature in the presence of a base, e.g. sodium hydride, in a suitable solvent, e.g. a dipolar aprotic solvent such as N,N-dimethylformamide.

Where the respective values of R⁵, R^{5a} and R^{5b} permit, a compound of formula (IA) may be obtained from the corresponding compound of formula (IF) by conventional catalytic hydrogenation, e.g. by treatment with gaseous hydrogen in the presence of a hydrogenation catalyst such as palladium on charcoal.

A compound containing the moiety —S— may be converted into the corresponding compound containing the moiety —S(O)— by treatment with 3-chloroperoxybenzoic acid. Likewise, a compound containing the moiety —S— or —S(O)— may be converted into the corresponding compound containing the moiety —S(O)₂— by treatment with 3-chloroperoxybenzoic acid.

A compound containing the moiety —S— may be converted into the corresponding compound containing the moiety —S(O)(NH)— by treatment with ammonium carbamate and (diacetoxyiodo)benzene.

Where a mixture of products is obtained from any of the processes described above for the preparation of compounds according to the invention, the desired product can be separated therefrom at an appropriate stage by conventional methods such as preparative HPLC; or column chromatography utilising, for example, silica and/or alumina in conjunction with an appropriate solvent system.

Where the above-described processes for the preparation of the compounds according to the invention give rise to mixtures of stereoisomers, these isomers may be separated by conventional techniques. In particular, where it is desired to obtain a particular enantiomer of a compound of formula (I) this may be produced from a corresponding mixture of enantiomers using any suitable conventional procedure for resolving enantiomers. Thus, for example, diastereomeric derivatives, e.g. salts, may be produced by reaction of a mixture of enantiomers of formula (I), e.g. a racemate, and an appropriate chiral compound, e.g. a chiral base. The diastereomers may then be separated by any convenient means, for example by crystallisation, and the desired enantiomer recovered, e.g. by treatment with an acid in the instance where the diastereomer is a salt. In another resolution process a racemate of formula (I) may be separated using chiral HPLC. Moreover, if desired, a particular enantiomer may be obtained by using an appropriate chiral intermediate in one of the processes described above. Alternatively, a particular enantiomer may be obtained by performing an enantiomer-specific enzymatic biotransformation, e.g. an ester hydrolysis using an esterase, and then purifying only the enantiomerically pure hydrolysed acid from the unreacted ester antipode. Chromatography, recrystallisation and other conventional separation procedures may also be used with intermediates or final products where it is desired to obtain a particular geometric isomer of the invention.

During any of the above synthetic sequences it may be necessary and/or desirable to protect sensitive or reactive groups on any of the molecules concerned. This may be achieved by means of conventional protecting groups, such as those described in Greene's Protective Groups in Organic Synthesis, ed. P. G. M. Wuts, John Wiley & Sons, 5^{th} edition, 2014. The protecting groups may be removed at any convenient subsequent stage utilising methods known from the art.

The compounds in accordance with this invention potently inhibit the ability of IL-17A to bind to IL-17RA. When tested in the IL-17 FRET assay described below, compounds of the present invention exhibit an IC₅₀ value of 10 μM or less, generally of 5 μM or less, usually of 1 μM or less, typically of 500 nM or less, suitably of 100 nM or less, ideally of 50 nM or less, and preferably of 25 nM or less (the skilled person will appreciate that a lower IC₅₀ figure denotes a more active compound).

Moreover, certain compounds in accordance with this invention potently inhibit IL-17 induced IL-6 release from human dermal fibroblasts. Indeed, when tested in the HDF cell line assay described below, compounds of the present invention exhibit an IC₅₀ value of 10 μM or less, generally of 5 μM or less, usually of 1 μM or less, typically of 500 nM or less, suitably of 100 nM or less, ideally of 50 nM or less, and preferably of 25 nM or less (as before, the skilled person will appreciate that a lower IC₅₀ figure denotes a more active compound).

### IL-17 FRET Assay

The purpose of this assay is to test the ability of compounds to disrupt the interaction between IL-17A and soluble IL-17 Receptor A (IL-17RA). The ability of a compound to inhibit IL-17A binding to IL-17RA is measured in this assay.

An IL-17AA-TEV-Human Fc construct was expressed in a CHO SXE cell system and purified by protein A chromatography and size exclusion. The protein was labelled with an amine reactive AlexaFluor 647 dye (Thermo Fisher #A20006), as per manufacturer's instruction.

Soluble IL-17RA (33-317)-HKH-TEV-Fc was expressed in an Expi HEK293 cell system and purified by protein A chromatography and size exclusion. The Fc tag was cleaved by TEV, producing IL-17RA (33-317)-HKH, and the protein was labelled with amine reactive terbium (Thermo Fisher #PV3581).

In assay buffer [Dulbecco's PBS (Sigma #14190-094), 0.05% P20 (Thermo Scientific #28320), 1 mg/mL BSA (Sigma #A2153-500G)] the following solutions were prepared:

### For IL-17A Assay

IL-17A-Fc-AF647 at 5 nM
IL-17RA-HKH—Tb at 5 nM

Compounds were serially diluted in DMSO before receiving an aqueous dilution into a 384 well dilution plate (Greiner #781281), to give a 25% DMSO solution.

IL-17A (10 μL) was added to a black low volume assay plate (Costar #4511) and diluted compound (5 μL) was transferred from the aqueous dilution plate. The cytokine and compound were allowed to incubate for 1 h, then IL-17RA (10 μL) was added. The plates were wrapped in foil and incubated at room temperature for 18-20 h with gentle shaking (<400 rpm) before being read on a Perkin Elmer Envision plate reader (Excitation: 330 nm; Emission 615/645 nm).

The final assay concentrations were IL-17A-AF647 2 nM and IL-17RA-Tb 2 nM, 5% DMSO.

When tested in the IL-17 FRET assay, the compounds of the accompanying Examples were all found to exhibit IC₅₀ values of 10 μM or better.

When tested in the IL-17 FRET assay, compounds of the accompanying Examples exhibit IC₅₀ values generally in the range of about 0.01 nM to about 10 μM, usually in the range of about 0.01 nM to about 5 μM, typically in the range of about 0.01 nM to about 1 μM, suitably in the range of about 0.01 nM to about 500 nM, appositely in the range of about 0.01 nM to about 100 nM, ideally in the range of about 0.01 nM to about 50 nM, and preferably in the range of about 0.01 nM to about 25 nM.

Inhibition of IL-17A Induced IL-6 Release from Dermal Fibroblast Cell Line

The purpose of this assay is to test the neutralising ability to IL-17 proteins, in a human primary cell system. Stimulation of normal human dermal fibroblasts (HDF) with IL-17 alone produces only a very weak signal but in combination with certain other cytokines, such as TNFα, a synergistic effect can be seen in the production of inflammatory cytokines, i.e. IL-6.

HDFs were stimulated with IL-17A (50 pM) in combination with TNF-α (25 pM). The resultant IL-6 response was then measured using a homogenous time-resolved FRET kit from Cisbio. The kit utilises two monoclonal antibodies, one labelled with Eu-Cryptate (Donor) and the second with d2 or XL665 (Acceptor). The intensity of the signal is proportional to the concentration of IL-6 present in the sample (Ratio is calculated by 665/620×104).

The ability of a compound to inhibit IL-17 induced IL-6 release from human dermal fibroblasts is measured in this assay.

HDF cells (Sigma #106-05n) were cultured in complete media (DMEM+10% FCS+2 mM L-glutamine) and maintained in a tissue culture flask using standard techniques. Cells were harvested from the tissue culture flask on the morning of the assay using TrypLE (Invitrogen #12605036). The TrypLE was neutralised using complete medium (45 mL) and the cells were centrifuged at 300×g for 3 minutes. The cells were re-suspended in complete media (5 mL) counted and adjusted to a concentration of 3.125×10⁴ cells/mL before being added to the 384 well assay plate (Corning #3701) at 40 μL per well. The cells were left for a minimum of three hours, at 37° C./5% CO₂, to adhere to the plate.

Compounds were serially diluted in DMSO before receiving an aqueous dilution into a 384 well dilution plate (Greiner #781281), where 5 μL from the titration plate was transferred to 45 μL of complete media and mixed to give a solution containing 10% DMSO.

Mixtures of TNFα and IL-17 cytokine were prepared in complete media to final concentrations of TNFα 25 pM/IL-17A 50 pM, then 30 μL of the solution was added to a 384 well reagent plate (Greiner #781281).

10 μL from the aqueous dilution plate was transferred to the reagent plate containing 30 μL of the diluted cytokines, to give a 2.5% DMSO solution. The compounds were incubated with the cytokine mixtures for one hour at 37° C. After the incubation, 10 μL was transferred to the assay plate, to give a 0.5% DMSO solution, then incubated for 18-20 h at 37° C./5% CO₂.

From the Cisbio IL-6 FRET kit (Cisbio #62IL6PEB) europium cryptate and Alexa 665 were diluted in reconstitution buffer and mixed 1:1, as per kit insert. To a white low volume 384 well plate (Greiner #784075) were added FRET reagents (10 μL), then supernatant (10 μL) was transferred from the assay plate to Greiner reagent plate. The mixture was incubated at room temperature for 3 h with gentle shaking (<400 rpm) before being read on a Synergy Neo 2 plate reader (Excitation: 330 nm; Emission: 615/645 nm). When tested in the above assay, compounds of the accompanying Examples were found to exhibit IC₅₀ values of 10 μM or better.

When tested in the above assay, compounds of the accompanying Examples exhibit IC₅₀ values generally in the range of about 0.01 nM to about 10 μM, usually in the range of about 0.01 nM to about 5 μM, typically in the range of about 0.01 nM to about 1 μM, suitably in the range of about 0.01 nM to about 500 nM, appositely in the range of about 0.01 nM to about 100 nM, ideally in the range of about 0.01 nM to about 50 nM, and preferably in the range of about 0.01 nM to about 25 nM.

The following Examples illustrate the preparation of compounds according to the invention.

### EXAMPLES

Abbreviations

| | |
|---|---|
| DCM: dichloromethane | DMF: N,N-dimethylformamide |
| MeOH: methanol | THF: tetrahydrofuran |
| DMSO: dimethyl sulfoxide | DIPEA: N,N-diisopropylethylamine |
| EtOH: ethanol | EtOAc: ethyl acetate |
| TFA: trifluoroacetic acid | |
| EDC.HCl: N-(3-dimethylaminopropyl)-N′- | |
| ethylcarbodiimide hydrochloride | |
| HATU: 2-(7-aza-1H-benzotriazol-1-yl)-1,1,3,3- | |
| tetramethyluronium hexafluorophosphate | |
| XPhos: 2-dicyclohexylphosphino-2′,4′,6′- | |
| triisopropylbiphenyl | |
| {Ir[dF(CF₃)ppy]₂(dtbpy)}PF₆: [4,4′-bis(1,1- | |
| dimethylethyl)-2,2′-bipyridine-N¹,N¹′]bis- | |
| {3,5-difluoro-2-[5-(trifluoromethyl)-2-pyridinyl- | |
| N]phenyl-C}iridium(III) hexafluoro-phosphate | |
| h: hour | r.t.: room temperature |
| M: mass | RT: retention time |
| HPEC: High Performance Liquid Chromatography | |
| LCMS: Liquid Chromatography Mass Spectrometry | |
| ES+: Electrospray Positive Ionisation | |
| LED: light-emitting diode | PTFE: poly(tetrafluoroethylene) |

### Analytical Conditions

Compounds were named with the aid of ACD/Name Batch (Network) version 11.01, and/or Accelrys Draw 4.2, and/or Elemental, Dotmatics, and/or Chemaxon.

All reactions involving air- or moisture-sensitive reagents were performed under a nitrogen atmosphere using dried solvents and glassware.

NMR spectra were recorded on a Bruker Avance III HD 500 MHz, 400 MHz, 300 MHz or 250 MHz spectrometer.

uPLC-MS

Performed on a Waters Acquity UPLC system coupled to a Waters Acquity PDA detector, an ELS detector and an MSD (Scan Positive: 150-850).

Method 1

Phenomenex Kinetex-XB, C18 (2.1×100 mm, 1.7 μm) column

Mobile Phase A: 0.1% formic acid in water

Mobile Phase B: 0.1% formic acid in acetonitrile

Gradient: Flow rate 0.6 mL/minute; column temperature 40° C.

| Time | A % | B % |
|---|---|---|
| 0.00 | 95.00 | 5.00 |
| 5.30 | 0.00 | 100.0 |
| 5.80 | 0.00 | 100.0 |
| 5.82 | 95.00 | 5.00 |
| 7.00 | 95.00 | 5.00 |

HPLC-MS

Performed on a Shimadzu LCMS-2010EV system coupled to SPD-M20A PDA and PL 2100 detectors.

Method 2

Phenomenex Kinetex Core-Shell C8 (2.1×50 mm, 5 μm) column, protected by a

Phenomenex ‘Security Guard’ column

Mobile Phase A: 0.1% formic acid in water

Mobile Phase B: 0.1% formic acid in acetonitrile

Gradient: Flow rate 1.2 mL/minute; column temperature 40° C.

| Time | A % | B % |
|---|---|---|
| 0.00 | 95.00 | 5.00 |
| 1.20 | 0.00 | 100.0 |
| 1.30 | 0.00 | 100.0 |
| 1.31 | 95.00 | 5.00 |

Performed on an Agilent 1200-6120 LC-MS system coupled to Detection (230 to 400 nm and 215 nm) and Mass Spec Detection Agilent 6120 Mass Spectrometer (ES) m/z 120 to 800.

Method 3

Waters X-Bridge C18 (2.1×20 mm, 2.5 μm) column

Mobile Phase A: 10 mM ammonium formate in water+0.1% formic acid

Mobile Phase B: acetonitrile+5% water+0.1% formic acid

Gradient: Flow rate 1 mL/minute

| Time | A % | B % |
|---|---|---|
| 0.00 | 95.00 | 5.00 |
| 1.50 | 5.00 | 95.00 |
| 2.25 | 5.00 | 95.00 |
| 2.50 | 95.00 | 5.00 |

Performed on a Waters ZQ system coupled to Waters 2996 PDA and Waters 2420 detectors.

Method 4

Phenomenex Gemini-NX C18 (2.0×50 mm, 3 μm) column

Mobile Phase A: 2 mM ammonium bicarbonate modified to pH 10 with NH₄OH

Mobile Phase B: acetonitrile

Gradient: Flow rate 1 mL/minute; column temperature 40° C.

| Time | A % | B % |
|---|---|---|
| 0.00 | 99.00 | 1.00 |
| 1.80 | 0.00 | 100.0 |
| 2.10 | 0.00 | 100.0 |
| 2.30 | 99.00 | 1.00 |
| 3.50 | 99.00 | 1.00 |

Automated Preparative Reverse Phase HPLC Purification

Performed using a Gilson system with a Gilson 331&332 pump, a Gilson GX281 autoinjector, a Gilson GX281 fraction collector and a Gilson 155&157 UV detector.

Method 5

X-Bridge C18 Waters (30×100 mm, 10 μm) column

Mobile Phase A: water+0.2% ammonia solution

Mobile Phase B: acetonitrile+0.2% ammonia solution

Gradient: Flow rate 40 mL/minute

| Time | A % | B % |
|---|---|---|
| 0.00 | 90 | 10 |
| 0.55 | 90 | 10 |
| 14.44 | 5 | 95 |
| 16.55 | 5 | 95 |
| 16.75 | 90 | 10 |

Performed using a Gilson system with a Gilson 331&332 pump, a Gilson GX281 autoinjector, a Gilson GX281 fraction collector and a Gilson 159 UV detector.

Method 6

Sunfire C18 Waters (30×100 mm, 10 μm) column

Mobile Phase A: water+0.1% formic acid

Mobile Phase B: acetonitrile+0.1% formic acid

Gradient: Flow rate 40 mL/minute

| Time | A % | B % |
|---|---|---|
| 0.00 | 90.00 | 10.00 |
| 0.55 | 90.00 | 10.00 |
| 11.00 | 5.00 | 95.00 |
| 13.10 | 5.00 | 95.00 |
| 13.31 | 90.00 | 10.00 |

Performed using an Agilent 1260-6120 LC-MS system, with an Agilent binary pump and Agilent DAD (240-400 nm) module. 6120 mass detection (ES) m/z 120-1000

Method 7

X-Bridge C18 (2.1×20 mm, 2.5 μm) column

Mobile Phase A: 10 nM ammonium formate in water+0.1% ammonium hydroxide

Mobile Phase B: acetonitrile+5% water+0.1% ammonium hydroxide

Gradient: Flow rate 1 mL/minute

| Time | A % | B % |
|---|---|---|
| 0.00 | 95.00 | 5.00 |
| 1.50 | 5.00 | 95.00 |
| 2.25 | 5.00 | 95.00 |
| 2.50 | 95.00 | 5.00 |

Performed using an Agilent 1200RR-6140 LC-MS system, with an Agilent binary pump and Agilent DAD (230-400 nm) module. 6140 mass detection (ES) m/z 100-1000

Method 8

X-Bridge C18 (2.1×20 mm, 2.5 μm) column

Mobile Phase A: 10 nM ammonium formate in water+0.1% ammonia solution

Mobile Phase B: acetonitrile+5% water+0.1% ammonia solution

Gradient: Flow rate 1 mL/minute

| Time | A % | B % |
|---|---|---|
| 0.00 | 95.10 | 5.00 |
| 4.00 | 5.00 | 95.00 |
| 5.00 | 5.00 | 95.00 |
| 5.10 | 95.10 | 5.00 |

Method 9

X-Bridge C18 (2.1×20 mm, 2.5 μm) column

Mobile Phase A: 10 nM ammonium formate in water+0.1% formic acid

Mobile Phase B: acetonitrile+5% water+0.1% formic acid

Gradient: Flow rate 1 mL/minute

| Time | A % | B % |
|---|---|---|
| 0.00 | 95.00 | 5.00 |
| 4.00 | 5.00 | 95.00 |
| 5.00 | 5.00 | 95.00 |
| 5.10 | 95.00 | 5.00 |

Performed using an Agilent 1290-MSD-XT LC-MS system, with an Agilent binary pump and Agilent DAD (230-400 nm) module. MSD-XT mass detection (ES) m/z 100-1000.

Method 10

Acquity UPLC BEH C18 (2.1×50 mm, 1.7 μm) column

Mobile Phase A: 10 mM ammonium formate in water+0.1% ammonia solution

Mobile Phase B: acetonitrile+5% water+0.1% ammonia solution

Gradient: Flow rate 1.5 mL/minute

| Time | A % | B % |
|---|---|---|
| 0.00 | 95.00 | 5.00 |
| 0.10 | 95.00 | 5.00 |
| 3.50 | 5.00 | 95.00 |
| 4.00 | 5.00 | 95.00 |
| 4.05 | 95.00 | 5.00 |

Method 11

Acquity UPLC BEH C18 (2.1×50 mm, 1.7 μm) column

Mobile Phase A: 10 mM ammonium formate in water+0.1% formic acid

Mobile Phase B: acetonitrile+5% water+0.1% formic acid

Gradient: Flow rate 1.5 mL/minute

| Time | A % | B % |
|---|---|---|
| 0.00 | 95.00 | 5.00 |
| 0.10 | 95.00 | 5.00 |
| 3.50 | 5.00 | 95.00 |
| 4.00 | 5.00 | 95.00 |
| 4.05 | 95.00 | 5.00 |

Performed using a QDA Waters simple quadrupole mass spectrometer with an ESI source and a UPLC Acquity Classic with diode array detector (210 to 400 nm). Data are acquired in a full MS scan from m/z 70 to 800 in positive/negative mode.

Method 12

Waters Acquity UPLC BEH C18 (2.1×50 mm, 1.7 μm) column

Mobile Phase A: water/acetonitrile/ammonium formate (95/5/63 mg/L)+100 μg/L NH₄OH

Mobile Phase B: acetonitrile/water/ammonium formate (95/5/63 mg/L)+100 μg/L NH₄OH

Gradient: Flow rate 0.4 mL/minute to 0.5 mL/minute

| Time | A % | B % |
|---|---|---|
| 0 | 99 | 1 |
| 0.3 | 99 | 1 |
| 3.2 | 0 | 100 |
| 3.25 | 0 | 100 |
| 4 | 0 | 100 |
| 4.1 | 99 | 1 |
| 4.8 | 90 | 1 |

Performed using a QDA Waters simple quadrupole mass spectrometer with an ESI source and a UPLC Acquity with diode array detector (200 to 400 nm). Data are acquired in a full MS scan from m/z 70 to 800 in positive/negative mode.

Method 13

Waters Acquity UPLC XSelect HSS T3 (2.1×50 mm, 1.8 μm) column

Mobile Phase A: water/acetonitrile/formic acid (95:5:0.05)

Mobile Phase B: acetonitrile/formic acid (99.95:0.05)

Gradient: Flow rate 0.4 mL/minute to 0.5 mL/minute

| Time | A % | B % |
|---|---|---|
| 0 | 99 | 1 |
| 0.3 | 99 | 1 |
| 3.2 | 5 | 95 |
| 3.25 | 5 | 95 |
| 4 | 5 | 95 |
| 4.1 | 99 | 1 |
| 5.5 | 99 | 1 |

Performed using a SYNAPT G2-SI Waters Q-TOF mass spectrometer for QC analysis, with an ESI source and a Waters Acquity H-class UPLC with diode array detector (210 to 400 nm). Data are acquired in a full MS scan from m/z 50 to 1200 in positive mode.

Method 14

Acquity UPLC HSS T3 C18 (1.8 μm, 2.1×50 mm) column

Solvent A: water/acetonitrile/formic acid (95/5/750 μg/L)

Solvent B: water/acetonitrile/formic acid (5/95/500 μg/L)

Gradient: Flow rate 0.5 mL/minute to 0.8 mL/minute

| Time | A % | B % |
|---|---|---|
| 0 | 98 | 2 |
| 0.3 | 98 | 2 |
| 3 | 5 | 95 |
| 4 | 5 | 95 |
| 4.1 | 98 | 2 |
| 5.1 | 98 | 2 |

Performed using a Waters I-Class UPLC system coupled to PDA and QDa MS detectors.

Method 15

Waters XBridge BEH C18 XP (2.5 μm, 2.1×50 mm) column

Mobile Phase A: 10 mM ammonium formate+0.1% NH₃ (pH 10)

Mobile Phase B: acetonitrile+5% water+0.1% NH₃ (pH 10)

Gradient: Flow rate 1 mL/minute

| Time | A % | B % |
|---|---|---|
| 0 | 95 | 5 |
| 0.1 | 95 | 5 |
| 2.6 | 5 | 95 |
| 2.75 | 5 | 95 |
| 2.8 | 95 | 5 |
| 3 | 95 | 5 |

Method 16

Waters XBridge BEH C18 XP (2.5 μm, 2.1×50 mm) column

Mobile Phase A: 10 mM ammonium formate+0.1% formic acid (pH 3)

Mobile Phase B: acetonitrile+5% water+0.1% formic acid (pH 3)

Gradient: Flow rate 1 mL/minute

| Time | A % | B % |
|---|---|---|
| 0 | 95 | 5 |
| 0.1 | 95 | 5 |
| 2.6 | 5 | 95 |
| 2.75 | 5 | 95 |
| 2.8 | 95 | 5 |
| 3 | 95 | 5 |

Performed using LCMS purification (Basic mode, LCMS prep) using SQD Waters single quadrupole mass spectrometer with an ESI source, Waters 2535 quaternary pump coupled with 2767 Sample Manager and with diode array detector (210 to 400 nm). Data are acquired in a full MS scan from m/z 100 to 850 in positive and negative modes with a basic elution.

Method 17

Waters XBridge OBD MS C18 (5 μm, 30×50 mm) column

Mobile Phase A: water+10 mM NH₄HCO₃+50 μg/L NH₄OH

Mobile Phase B: acetonitrile

Mobile Phase D: water+100 mM NH₄HCO₃+500 μg/L NH₄OH (pH˜8.5)

Gradient: Flow rate 35 mL/minute to 45 mL/minute

| Time | A % | B % | D % |
|---|---|---|---|
| 0 | 85 | 5 | 10 |
| 1 | 85 | 5 | 10 |
| 7 | 10 | 85 | 5 |
| 9 | 10 | 85 | 5 |
| 12 | 10 | 85 | 5 |

Performed on a Shimadzu LCMS-2010EV system coupled to SPD-M20A PDA and Softa Model 400 ELS detectors.

Method 18

Waters XBridge C18 (50 mm×3.0 mm, 2.5 μm) column

Mobile Phase A: 5 mM ammonium bicarbonate in water

Mobile Phase B: acetonitrile

Gradient program: Flow rate 1.2 mL/minute; column oven: 50° C.

| Time | A % | B % |
|---|---|---|
| 0.0 | 100 | 0 |
| 2.0 | 5 | 95 |
| 3.0 | 5 | 95 |
| 3.2 | 100 | 0 |
| 4.0 | 100 | 0 |

Performed on a Shimadzu LCMS-2010EV system coupled to SPD-M20A PDA and PL 2100 detectors.

Method 19

Waters Atlantis dC18 (2.1×100 mm, 3 μm) column

Mobile Phase A: 0.1% formic acid in water

Mobile Phase B: 0.1% formic acid in acetonitrile

Gradient program: Flow rate 0.6 mL/minute; column temperature 40° C.

| Time | A % | B % |
|---|---|---|
| 0.00 | 95.00 | 5.00 |
| 5.00 | 0.00 | 100.0 |
| 5.40 | 0.00 | 100.0 |
| 5.42 | 95.00 | 5.00 |

Method 20

Waters Acquity UPLC BEH C18 (2.1×50 mm, 1.7 μm) column

Solvent A: 10 mM ammonium formate in water+0.1% formic acid

Solvent B: acetonitrile+5% water+0.1% formic acid

Gradient: Flow rate 0.7 mL/minute

| Time | A % | B % |
|---|---|---|
| 0.00 | 95.00 | 5.00 |
| 4.00 | 5.00 | 95.00 |
| 5.00 | 5.00 | 95.00 |
| 5.10 | 95.00 | 5.00 |

Performed using a Waters I-Class UPLC system coupled to PDA and QDa MS detectors.

Method 21

X-Bridge C18 (2.1×20 mm, 2.5 μm) column

Mobile Phase A: 10 mM ammonium formate in water+0.1% ammonia solution

Mobile Phase B: acetonitrile+5% water+0.1% ammonia solution

Gradient program: Flow rate 1 mL/minute

| Time | A % | B % |
|---|---|---|
| 0.00 | 95.00 | 5.00 |
| 1.50 | 5.00 | 95.00 |
| 2.25 | 5.00 | 95.00 |
| 2.50 | 95.00 | 5.00 |

Method 22

Waters Thar 3100 SFC system connected to a Waters 2998 PDA detector

Column: Amylose-2 25 cm

Isocratic eluent: 80% heptane-20% 2-propanol at 1 mL/minute

Method 23

Waters Thar 3100 SFC system connected to a Waters 2998 PDA detector

Column: Chiralpak AS-H 25 cm

Isocratic eluent: 10% methanol-90% CO₂ at 4 mL/minute

LC-MS (Basic Method)

Performed on a Shimadzu LCMS-2010EV system coupled to SPD-M20A PDA and Softa Model 400 ELS detectors.

Method 24

Waters XBridge C18 (30 mm×2.1 mm, 2.5 μm) column

Mobile Phase A: 0.1% ammonia in 5 mM ammonium formate buffer

Mobile Phase B: 0.1% NH₃ in acetonitrile/5 mM ammonium formate buffer (95:5)

Gradient program: Flow rate 1.0 mL/minute

| Time | A % | B % |
|---|---|---|
| 0.0 | 95 | 5 |
| 4.0 | 5 | 95 |
| 5.0 | 5 | 95 |
| 5.1 | 95 | 5 |
| 6.5 | 95 | 5 |

### Intermediate 1

### Methyl 2-cyclooctylidene-2-formamidoacetate

A solution of potassium tert-butoxide in THF (1M, 48 mL, 48 mmol) was added dropwise to a solution of methyl isocyanoacetate (4.0 mL, 41.8 mmol) in anhydrous THF (40 mL) at approximately −65° C. under nitrogen. After stirring for 5 minutes, a solution of cyclooctanone (5 g, 39.62 mmol) in anhydrous THF (20 mL) was added slowly at −70° C. The reaction mixture was stirred at −70° C. for 30 minutes, then the cooling bath was removed and the mixture was allowed to warm to 20° C. with stirring under nitrogen for 60 h. The resultant deep red solution was quenched with water (100 mL) and stirred at 20° C. for 1 h. The residue was extracted with ethyl acetate (3×100 mL). The combined organic extracts were washed with brine (50 mL) and dried over magnesium sulfate, then filtered and concentrated in vacuo. The resulting crude viscous orange oil was separated by flash column chromatography, using a gradient of ethyl acetate in heptane (0-90%), to afford the title compound (5.37 g, 58%) as a viscous orange oil, which solidified upon standing. δ_{H} (500 MHz, DMSO-d₆) 9.31 (s, 1H), 8.01 (d, J 1.5 Hz, 1H), 3.60 (s, 3H), 2.52-2.47 (m, 2H), 2.31-2.23 (m, 2H), 1.74-1.60 (m, 4H), 1.50-1.31 (m, 6H). HPLC-MS (method 7): MNa+ m/z 248, RT 1.63 minutes.

### Intermediate 2

### Methyl 2-cyclooctyl-2-formamidoacetate

Magnesium turnings (3.15 g, 129.6 mmol) were added carefully to a stirred solution of Intermediate 1 (2.91 g, 12.95 mmol) in anhydrous methanol (65 mL) at 0° C. under nitrogen. The suspension was stirred at 0° C. for 1 h, then allowed to warm to 20° C. over 2 h. Stirring of the turbid suspension was continued at 20° C. for 16 h. An additional portion of magnesium turnings (1 g, 41.14 mmol) was added, and the suspension was stirred at 20° C. for 3.5 h under nitrogen. The mixture was carefully concentrated in vacuo. The residue was suspended in ethyl acetate (100 mL) and water (200 mL), then cooled to 0° C. The mixture was treated with aqueous hydrochloric acid to aid dissolution of the solids (approx. pH 1). The layers were separated, and the aqueous layer was further extracted with ethyl acetate (3×100 mL). The combined organic extracts were washed with brine (50 mL) and dried over magnesium sulfate, then filtered and concentrated in vacuo. The resulting crude orange viscous oil was separated by flash column chromatography, using a gradient of ethyl acetate in heptane (0-80%), to afford the title compound (1.53 g, 48%) as a viscous orange oil. Major rotamer: δ_{H} (500 MHz, DMSO-d₆) 8.46 (d, J 8.5 Hz, 1H), 8.06 (s, 1H), 4.29 (dd, J 8.6, 6.1 Hz, 1H), 3.64 (s, 3H), 2.04-1.93 (m, 1H), 1.73-1.19 (m, 14H). HPLC-MS (method 19): MH+ m/z 228, RT 3.94 minutes.

### Intermediate 3

### Methyl 2-amino-2-cyclooctylacetate hydrochloride

Acetyl chloride (1.9 mL, 26.7 mmol) was added cautiously at 0° C. to a stirred solution of Intermediate 2 (1.54 g, 6.77 mmol) in methanol (68 mL) under nitrogen. After stirring for 5 minutes, the solution was heated at 50° C. for 2 h, then the mixture was concentrated in vacuo. The resulting crude orange powder was triturated from diethyl ether (40 mL) and the solids were collected by filtration, washing with diethyl ether (2×20 mL). The solids were dried in vacuo at 50° C. for 6 h to afford the title compound (1.43 g, 81%) as a tan powder. δ_{H} (500 MHz, DMSO-d₆) 8.61 (br s, 3H), 3.86 (d, J 4.4 Hz, 1H), 3.73 (s, 3H), 2.19-2.09 (m, 1H), 1.68-1.37 (m, 13H), 1.32-1.20 (m, 1H).

### Intermediate 4

### Methyl 2-cyclooctyl-2-[(3-methylisoxazole-4-carbonyl)amino]acetate

To a solution of 3-methyl-4-isoxazolecarboxylic acid (1.65 g, 12.7 mmol) and Intermediate 3 (3 g, 12.73 mmol) in DMF (20 mL) at 0° C. was added HATU (5.99 g, 15.3 mmol), followed by DIPEA (8.9 mL, 51 mmol, 8.9 mL). The mixture was stirred overnight, with warming to r.t., then diluted with water (100 mL) and extracted with EtOAc (2×80 mL). The organic extracts were dried over sodium sulfate, then filtered and evaporated to dryness. The residue was purified by flash chromatography, using a gradient of EtOAc/hexanes (10-80%), to yield the title compound (3.85 g, 98%) as a tan oil. δ_{H} (400 MHz, CDCl₃) 8.77 (d, J 0.7 Hz, 1H), 6.32 (d, J 8.6 Hz, 1H), 4.72 (dd, J 8.6, 4.8 Hz, 1H), 3.80 (s, 3H), 2.54 (d, J 0.6 Hz, 3H), 2.28-2.12 (m, 1H), 1.83-1.32 (m, 14H). HPLC-MS (method 7): [M+H]⁺ m/z 309, RT 1.37 minutes.

### Intermediate 5

### 2-Cyclooctyl-2-[(3-methylisoxazole-4-carbonyl)amino]acetic acid

To a solution of Intermediate 4 (3.85 g, 12.5 mmol) in THF (40 mL) was added a solution of lithium hydroxide monohydrate (786 mg, 18.7 mmol) in water (10 mL). The reaction mixture was stirred for 72 h at r.t., then diluted with water (30 mL) and acidified to pH 3 with 2N aqueous HCl (approx. 10 mL). The material was extracted with EtOAc (50 mL). The aqueous extracts were dried over sodium sulfate, then filtered and evaporated. To the resulting beige oil was added acetonitrile (50 mL) with stirring. The precipitated solid was filtered and dried on a sintered funnel for 2 h to give the title compound (560 mg). The mother liquor was concentrated. To the resulting white slurry was added diethyl ether (60 mL) with stirring, then the mixture was concentrated. The resultant gum was lyophilised from acetonitrile (10 mL) and water (30 mL) to give another crop of the title compound (1.7 g). δ_{H} (400 MHz, CDCl₃) 8.79 (s, 1H), 6.25 (d, J 8.5 Hz, 1H), 4.78 (dd, J 8.5, 4.5 Hz, 1H), 2.55 (s, 3H), 2.28 (s, 1H), 1.86-1.36 (m, 14H). HPLC-MS (method 21): [M+H]⁺ m/z 295, RT 0.91 minutes.

### Intermediate 6

### Methyl 2-[(tert-butoxycarbonyl)amino]-2-cyclooctylacetate

To Intermediate 3 (40 g, 0.17 mol) dissolved in DCM (500 mL), at 0° C., were added triethylamine (68.4 g, 0.68 mol) and di-tert-butyl dicarbonate (38.8 g, 0.18 mol). The reaction mixture was stirred at r.t. for 20 h, then diluted with water (400 mL) and extracted with DCM (2×500 mL). The organic layer was washed with brine (400 mL) and dried over sodium sulfate, then filtered and concentrated in vacuo. The crude residue was triturated with petroleum ether to afford the title compound (45.2 g, 89%) as a white solid. δ_{H} (250 MHz, DMSO-d₆) 7.14 (d, J 8.6 Hz, 1H), 4.03-3.78 (m, 1H), 3.61 (s, 3H), 2.02-1.84 (m, 1H), 1.75-1.19 (m, 23H).

### Intermediate 7

### 2-[(tert-Butoxycarbonyl)amino]-2-cyclooctylacetic acid

Lithium hydroxide monohydrate (75 mg, 1.78 mmol) was added to a stirred solution of Intermediate 6 (485 mg, 1.62 mmol) in 2:1 THF-water (12 mL). The reaction mixture was stirred at 20° C. for 15 h, then concentrated and dried in vacuo for 2 h. The resulting crude material (471 mg) was suspended in EtOAc (20 mL) and treated with saturated aqueous ammonium chloride solution (20 mL), followed by aqueous hydrochloric acid (5 mL). The layers were separated, and the aqueous layer was extracted with EtOAc (2×30 mL). The combined organic extracts were washed with brine (50 mL) and dried over sodium sulfate, then filtered and concentrated in vacuo, to give the title compound as a white powder. δ_{H} (400 MHz, CDCl₃) 4.97 (d, J 9.1 Hz), 4.24 (dd, J 9.2, 4.5 Hz), 2.14 (s, 2H), 1.45 (s, 21H).

### Intermediate 8

### 4-(4-Bromo-2-methylphenyl)oxane-4-carbonitrile

Sodium bis(trimethylsilyl)amide solution in THF (1M, 19.5 mL, 19.5 mmol) was added dropwise to a solution of 2-(4-bromo-2-methylphenyl)acetonitrile (3.75 g, 17.85 mmol) in THF (90 mL) at 0° C. After stirring for 0.5 h, the cooling bath was removed and the reaction mixture was stirred at 20° C. for 0.5 h. 1-Iodo-2-(2-iodoethoxy)ethane (2.8 mL, 19.67 mmol) was added dropwise. The reaction mixture was stirred for 0.5 h at 20° C. Sodium bis(trimethylsilyl)amide solution in THF (1M, 19.5 mL, 19.5 mmol) was added dropwise. The reaction mixture was stirred for 18 h at 20° C., then quenched with saturated aqueous ammonium chloride solution (25 mL) and diluted with water (25 mL). The aqueous layer was extracted with EtOAc (3×50 mL). The combined organic extracts were washed with brine (50 mL) and dried over sodium sulfate, then filtered and concentrated in vacuo. The resulting brown oil was purified by flash column chromatography, using a gradient of tert-butyl methyl ether in heptane (0-25%), to afford the title compound (2.3 g, 45%) as a yellow solid. δ_{H} (250 MHz, CDCl₃) 7.47-7.36 (m, 2H), 7.16 (d, J 8.4 Hz, 1H), 4.16-4.06 (m, 2H), 4.06-3.91 (m, 2H), 2.65 (s, 3H), 2.33-2.21 (m, 2H), 2.17-1.99 (m, 2H). HPLC-MS (method 9): [M+water]+ m/z 297 and 299, RT 1.80 minutes.

### Intermediate 9

### tert-Butyl N-[4-(4-cyanooxan-4-yl)-3-methylphenyl]carbamate

A sealable tube was charged with Intermediate 8 (200 mg, 0.71 mmol), tert-butyl carbamate (167 mg, 1.43 mmol) and cesium carbonate (395 mg, 1.21 mmol). The reagents were suspended in toluene (2 mL). The reaction mixture was charged with palladium(II) acetate (4.8 mg, 21.4 μmol) and XPhos (20.4 mg, 42.8 μmol), then purged with nitrogen and sonicated for 5 minutes. The reaction vessel was sealed and heated at 90° C. for 3 h. The reaction mixture was quenched with water (10 mL), then extracted with EtOAc (20 mL) and filtered. The layers were separated, and the aqueous layer was extracted with EtOAc (20 mL). The combined organic extracts were washed with brine (10 mL) and dried over sodium sulfate, then filtered and concentrated in vacuo. The residue was separated by column chromatography, using a gradient of tert-butyl methyl ether/heptane (0-50%), to afford the title compound (237 mg, 95%) as a beige solid. δ_{H} (250 MHz, CDCl₃) 7.31-7.27 (m, 1H), 7.26-7.16 (m, 2H), 6.45 (s, 1H), 4.14-4.05 (m, 2H), 4.05-3.92 (m, 2H), 2.61 (s, 3H), 2.34-2.20 (m, 2H), 2.16-1.98 (m, 2H), 1.52 (s, 9H).

HPLC-MS (method 4): [M+H]⁺ m/z 317, RT 1.79 minutes.

### Intermediate 10

### 4-(4-Amino-2-methylphenyl)oxane-4-carbonitrile

TFA (0.8 mL, 10.5 mmol) was added to a solution of Intermediate 9 (90% purity, 0.24 g, 0.68 mmol) in DCM (5 mL). The reaction mixture was stirred for 5 h at 20° C., then quenched with saturated aqueous sodium hydrogen carbonate solution (20 mL) and stirred for 15 minutes at 20° C. The layers were separated. The aqueous layer was extracted with DCM (2×15 mL). The combined organic extracts were filtered using a hydrophobic frit, and the solvent was concentrated in vacuo, to afford the title compound (0.15 g, 99%) as a brown solid. δ_{H} (250 MHz, CDCl₃) 6.96 (d, J 8.3 Hz, 1H), 6.52-6.41 (m, 2H), 4.04-3.94 (m, 2H), 3.94-3.82 (m, 2H), 3.60 (br s, 2H), 2.47 (s, 3H), 2.23-2.09 (m, 2H), 2.05-1.88 (m, 2H). HPLC-MS (method 2): [M+H]⁺ m/z 217, RT 0.73 minutes.

### Intermediate 11

### 4-(4-Bromo-2-methylphenyl)oxane-4-carboxamide

Potassium hydroxide (78.5 mg, 1.40 mmol) was added to a solution of Intermediate 8 (100 mg, 0.35 mmol) in ethylene glycol (2 mL) and water (0.4 mL). The reaction mixture was heated at 120° C. for 20 h. An additional portion of potassium hydroxide (109 mg, 1.94 mmol) was added, and heating was recommenced at 140° C. for 3 h. The reaction mixture was cooled to 20° C., then water (5 mL) was added and the aqueous layer was extracted with diethyl ether (2×20 mL). The combined organic extracts were washed with water (5 mL) and brine (5 mL), then dried over magnesium sulfate and filtered. The solvent was concentrated in vacuo, and azeotroped with heptane, to afford the title compound (107 mg, 98%) as a sticky cream-coloured solid. δ_{H} (250 MHz, CDCl₃) 7.36-7.26 (m, 2H), 7.25-7.20 (m, 1H), 5.23 (br s, 1H), 4.94 (br s, 1H), 4.00-3.85 (m, 2H), 3.74-3.61 (m, 2H), 2.38-2.22 (m, 2H), 2.28 (s, 3H), 2.06-1.91 (m, 2H). HPLC-MS (method 2): [M+H]⁺ m/z 298 and 300, RT 1.01 minutes.

### Intermediate 12

### 4-(4-Bromo-2-methylphenyl)-N,N-dimethyloxane-4-carboxamide

Intermediate 11 (95% purity, 107 mg, 0.34 mmol) in THF (2 mL) was added dropwise to a suspension of sodium hydride (60% purity, 40.9 mg, 1.02 mmol) in THF (1 mL) at 0° C. The reaction mixture was stirred at 0° C. for 1 h, then iodomethane (63.7 μL, 1.02 mmol) was added. The reaction mixture was warmed to 20° C. and stirred for 18 h, then cooled to 0° C. and quenched with water (2 mL). The mixture was warmed to 20° C., and was extracted with diethyl ether (2×10 mL). The organic extracts were washed with brine (5 mL) and dried over magnesium sulfate, then filtered and concentrated in vacuo. The oily residue was separated by flash column chromatography, using a gradient of tert-butyl methyl ether/heptane (0-100%), to afford the title compound (88 mg, 73%) as a white solid. δ_{H} (250 MHz, CDCl₃) 7.32-7.26 (m, 1H), 7.24-7.20 (m, 2H), 4.00-3.85 (m, 2H), 3.83-3.71 (m, 2H), 2.86 (s, 3H), 2.42 (s, 3H), 2.27-2.13 (m, 2H), 2.17 (s, 3H), 2.05-1.85 (m, 2H). HPLC-MS (method 2): [M+H]⁺ m/z 326 and 328, RT 1.15 minutes.

### Intermediate 13

### tert-Butyl N-{4-[4-(dimethylcarbamoyl)oxan-4-yl]-3-methylphenyl}carbamate

A sealable tube was charged with Intermediate 12 (83% purity, 95.9 mg, 0.24 mmol), tert-butyl carbamate (57 mg, 0.49 mmol) and cesium carbonate (135 mg, 0.41 mmol). The reagents were suspended in toluene (1 mL). The reaction mixture was charged with palladium(II) acetate (1.6 mg, 7.3 μmol) and XPhos (7.0 mg, 14.6 μmol), then purged with nitrogen and sonicated for 5 minutes. The reaction vessel was sealed and heated at 90° C. for 3 h. The reaction mixture was cooled to 20° C. and quenched with water (10 mL), then extracted with EtOAc (10 mL) and filtered. The layers were separated. The aqueous layer was extracted with EtOAc (10 mL). The combined organic extracts were washed with brine (5 mL) and dried over sodium sulfate, then filtered and concentrated in vacuo. The resulting orange oil was purified by column chromatography, using a gradient of tert-butyl methyl ether/heptane (0-100%), to afford the title compound (68 mg, 61%) as a pale yellow solid. δ_{H} (250 MHz, CDCl₃) 7.38-7.32 (m, 1H), 7.27-7.13 (m, 2H), 6.40 (s, 1H), 4.12-3.94 (m, 2H), 3.91-3.79 (m, 2H), 2.92 (br s, 3H), 2.49 (br s, 3H), 2.36-2.20 (m, 2H), 2.22 (s, 3H), 2.17-1.94 (m, 2H), 1.52 (s, 9H). HPLC-MS (method 4): [M+H]⁺ m/z 363, RT 1.69 minutes.

### Intermediate 14

### 4-(4-Amino-2-methylphenyl)-N,N-dimethyloxane-4-carboxamide

TFA (0.17 mL, 2.25 mmol) was added to a solution of Intermediate 13 (86%, 68 mg, 0.16 mmol) in DCM (2 mL). The reaction mixture was stirred for 3 h at 20° C., then quenched with saturated aqueous sodium hydrogen carbonate solution (5 mL) and stirred for 15 minutes at 20° C. The layers were separated, and the aqueous layer was extracted with DCM (2×10 mL). The combined organic extracts were washed with saturated aqueous sodium hydrogen carbonate solution (5 mL) and filtered using a hydrophobic frit, then the solvent was concentrated in vacuo, to afford the title compound (46.9 mg, 97%) as a brown solid. δ_{H}(250 MHz, CDCl₃) 7.19 (d, J 8.4 Hz, 1H), 6.58 (dd, J 8.5, 2.6 Hz, 1H), 6.50 (d, J 2.5 Hz, 1H), 4.12-3.92 (m, 2H), 3.91-3.77 (m, 2H), 2.94 (br s, 3H), 2.55 (br s, 3H), 2.36-2.23 (m, 2H), 2.18 (s, 3H), 2.12-1.94 (m, 2H), 1.81-1.43 (m, 2H). HPLC-MS (method 2): [M+H]⁺ m/z 263, RT 0.55 minutes.

### Intermediate 15

### 4-{5-Chlorobicyclo[4.2.0]octa-1(6),2,4-trien-7-ylidene}-2-(3-methylisoxazol-4-yl)-4,5-dihydro-1,3-oxazol-5-one

To a stirred solution of 2[(3-methylisoxazole-4-carbonyl)amino]acetic acid (44.2 g, 240 mmol) in anhydrous DCM (440 mL) was added EDC.HCl (59.8 g, 312 mmol) portion wise. The reaction mixture was stirred at ambient temperature for 1.5 h, then diluted with DCM (200 mL) and quenched with water (500 mL). The organic layer was separated and washed with brine (2×500 mL), then dried over anhydrous sodium sulfate and filtered. The solvent was concentrated in vacuo to afford 2-(3-methylisoxazol-4-yl)-4H-oxazol-5-one (34 g) as a yellow solid, which was utilised without further purification. δ_{H} (400 MHz, CDCl₃) 8.83 (s, 1H), 4.37 (s, 2H), 2.56 (s, 3H).

Titanium tetrachloride in DCM (1M, 4.8 mL, 4.80 mmol) was added to anhydrous THF (9 mL) at 0° C. A solution of 2-(3-methylisoxazol-4-yl)-4H-oxazol-5-one (0.2 g, 1.20 mmol) in anhydrous THF (1.5 mL) and a solution of 5-chlorobicyclo[4.2.0]octa-1,3,5-trien-7-one (0.2 g, 1.32 mmol) in anhydrous THF (1.5 mL) were added dropwise sequentially. The reaction mixture was stirred at 0° C. for 20 minutes. Anhydrous pyridine (0.78 mL, 14.47 mmol) was added dropwise at 0° C. over 30 minutes. The reaction mixture was stirred at 0° C. for a further 2 h, then at 20° C. for 16 h. The reaction mixture was quenched by the addition of saturated aqueous ammonium chloride solution (12 mL), and was stirred for a further 10 minutes. The solution was extracted with EtOAc (2×20 mL). The combined organic extracts were washed with brine (20 mL) and dried over magnesium sulfate, then filtered and concentrated in vacuo. The residue was separated by column chromatography, using a gradient of EtOAc/heptane (0-100%), to afford the title compound (311 mg, 83%) as a yellow solid. δ_{H} (500 MHz, DMSO-d₆) 9.73 (s, 1H), 7.54 (dd, J 8.1, 7.2 Hz, 1H), 7.48 (d, J 8.0 Hz, 1H), 7.39 (d, J 7.0 Hz, 1H), 4.06 (s, 2H), 2.60 (s, 3H). HPLC-MS (method 3): [M+H]⁺ m/z 301 and 303, RT 1.99 minutes.

### Intermediate 16

### tert-Butyl N-(4-bromo-3-fluorophenyl)carbamate

4-Bromo-3-fluoroaniline (2.48 g, 12.7 mmol) was suspended in water (20 mL) and treated with di-tert-butyl dicarbonate (3.42 g, 15.2 mmol) portionwise. The resulting suspension was stirred rapidly at r.t. for 40 h. The thick off-white suspension was diluted with water (20 mL) and stirred for 20 minutes, then filtered through a sintered funnel. The isolated solid was washed with water (2×10 mL), then dried under suction for 45 minutes, to yield the title compound (3.46 g, 94%) as an off-white solid. δ_{H} (400 MHz, CDCl₃) 7.44-7.37 (m, 2H), 6.90 (ddd, J 8.7, 2.5, 1.0 Hz, 1H), 6.51 (s, 1H), 1.52 (s, 9H). HPLC-MS (method 7): [M−^{t}Bu+H]⁺ m/z 234 and 236, RT 1.22 minutes.

### Intermediate 17

### 3-Fluoro-4-(tetrahydropyran-4-yl)aniline

In a capped vial, nickel chloride dimethoxyethane adduct (5.4 mg, 0.024 mmol) and 4,4′-di-tert-butyl-2,2′-dipyridyl (8 mg, 0.029 mmol) were suspended in anhydrous 1,2-dimethoxyethane (2 mL). Nitrogen gas was bubbled through the suspension, which was stirred for 10 minutes. In a second vial, Intermediate 16 (75 mg, 0.26 mmol) and {Ir[dF(CF₃)ppy]₂(dtbpy)}PF₆ (3 mg, 2.6 μmol) were dissolved in anhydrous 1,2-dimethoxyethane (2.4 mL) under a gentle stream of nitrogen, then 4-bromotetrahydropyran (44 μL, 0.26 mmol), 2,6-lutidine (62 μL, 0.527 mmol) and tris(trimethylsilyl)silane (80 μL, 0.26 mmol) were added. A portion of the nickel chloride/dipyridyl solution (0.1 mL) was added, and the second vial was sparged with nitrogen for 15 minutes. The resulting mixture was sealed and stirred at ambient temperature, whilst undergoing irradiation with a blue LED (450 nm) for 1 h. The residue was purified by column chromatography, using a gradient of 0-100% EtOAc/isohexane. The resulting crude tert-butyl N-[3-fluoro-4-(tetrahydropyran-4-yl)phenyl]carbamate (54 mg) was dissolved in DCM (2 mL), treated with TFA (0.5 mL) and stirred at r.t. for 1 h. The reaction mixture was concentrated in vacuo. The residue was diluted with DCM (5 mL) and washed with saturated aqueous sodium hydrogen carbonate solution (10 mL). The aqueous layer was re-extracted twice with DCM. The organic layers were combined, then filtered through a PTFE phase separator cartridge and concentrated in vacuo. The crude residue was purified by column chromatography, using a gradient of 0-100% EtOAc/isohexane, to yield the title compound (25 mg, 48%). HPLC-MS (method 7): [M+H]⁺ m/z 196, RT 1.15 minutes.

### Intermediate 18

### trans-(4-Methylcyclohexyl)methanol

To a cold (−20° C. to −5° C.) solution of trans-4-methylcyclohexanecarboxylic acid (68.5 g, 0.481 mol) in THF (550 mL) was added a solution of lithium aluminum hydride (2.4M in THF, 200 mL, 0.48 mol) slowly over circa 1 h. The mixture was stirred at −20° C. for 1.5 h, then allowed to warm to ambient temperature. The mixture was re-cooled in an ice-salt bath before water (16 mL), aqueous sodium hydroxide solution (15 wt %, 16 mL), and water (40 mL) were slowly and cautiously added. The resulting viscous mixture was stirred for 10 minutes, then diethyl ether (500 mL) was added. The resulting suspension was filtered through a pad of kieselguhr. The solvents were evaporated under reduced pressure to afford the title compound (63.5 g, 100%) as a clear, colourless mobile oil. δ_{H} (500 MHz, CDCl₃) 3.44 (d, J 6.3 Hz, 2H), 1.79-1.69 (m, 4H), 1.47-1.23 (m, 3H), 1.04-0.89 (m, 4H), 0.88 (d, J 6.6 Hz, 3H).

### Intermediate 19

### trans-4-Methylcyclohexanecarbaldehyde

To a cold (−10° C. to −5° C.) solution of Intermediate 18 (30.31 g, 0.229 mol) in DCM (250 mL), DIPEA (122 mL, 1.15 mol) and DMSO (81.4 mL, 0.688 mol) was added solid pyridine-sulfur trioxide complex (73 g, 0.458 mol) portionwise, maintaining the internal temperature below 20° C. The reaction mixture was stirred at ambient temperature for 16 h, then washed in turn with aqueous citric acid (1M, 200 mL) and brine (200 mL). The organic layer was filtered through phase separating filter paper. The solvent was removed under reduced pressure to afford the title compound (34.9 g, 100%) as a pale yellow oil. δ_{H} (250 MHz, CDCl₃) 9.61 (d, J 1.6 Hz, 1H), 2.28-2.03 (m, 1H), 1.95 (m, 2H), 1.80 (m, 2H), 1.56-1.14 (m, 3H), 1.07-0.80 (m, 5H, including the Me signal at δ 0.90 (d, J 6.5 Hz)).

### Intermediate 20

### (S)-4-Methyl-N-[(1E)-(trans-4-methylcyclohexyl)methylidene]benzenesulfinamide

To a solution of Intermediate 19 (34.9 g, 229 mmol) and (S)-4-methylbenzenesulfinamide (35.6 g, 229 mmol) in DCM (1.2 L) was added titanium(IV) ethoxide (85-90% purity, 174.5 g, 160 mL). The resulting solution was heated at reflux for 2 h. The reaction mixture was cooled to ambient temperature, then water (300 mL) was added slowly. The resulting thick paste was filtered through a pad of kieselguhr, then rinsed with DCM (300 mL) and water (300 mL). The two phases were separated. The DCM phase was dried over anhydrous sodium sulfate and filtered, then the solvent was evaporated, to give the title compound (55.7 g, 78%) as a yellow oil, which partially solidified upon standing. δ_{H} (250 MHz, CDCl₃) 8.11 (d, J 4.9 Hz, 1H), 7.70-7.49 (m, 2H), 7.29 (m, 2H), 2.40 (s, 2H), 2.38-2.24 (m, 1H), 2.06-1.66 (m, 4H), 1.53-1.16 (m, 4H), 1.07-0.91 (m, 2H), 0.89 (d, J 6.5 Hz, 3H).

### Intermediate 21

### N—[(S)-Cyano(trans-4-methylcyclohexyl)methyl]-(S)-4-methylbenzenesulfinamide

To a solution of diethylaluminium cyanide (1M in toluene, 103 mL, 103 mmol) in THF (400 mL) at −78° C. was added anhydrous isopropyl alcohol (5.3 mL, 69 mmol). The mixture was stirred at −78° C. for 30-60 minutes, then cannulated into a solution of Intermediate 20 (90% purity, 20.2 g, 69 mmol) in THF (800 mL) at −78° C. over circa 45 minutes. The mixture was allowed to warm to room temperature, then stirred overnight. The mixture was cooled in an ice-water bath, then saturated aqueous ammonium chloride solution (300 mL) was added; some gas was evolved and the internal temperature increased to circa 30° C. After 1 h, the mixture was filtered through a pad of kieselguhr, then the pad was washed with water (300 mL) and ethyl acetate (300 mL). The organic layers were divided, and the aqueous layers were washed with more ethyl acetate. The combined organic layers were washed with brine, dried over anhydrous sodium sulfate and filtered, then the solvent was evaporated. The resulting pale yellow oil, which solidified upon standing, was taken up in hot heptane-ethyl acetate, then allowed to crystallise, to afford the title compound (7.78 g, 38%) as a white solid. The residues were evaporated and purified by automated column chromatography to give a clean mixture of the two diastereoisomers. Recrystallisation of this mixture from ethyl acetate-heptane, seeded using some of the first crop, gave a further batch of the title compound (4.05 g, 20%). δ_{H} (250 MHz, CDCl₃) 7.61 (d, J 8.3 Hz, 2H), 7.36 (d, J 8.2 Hz, 3H), 4.50 (d, J 7.8 Hz, 1H), 3.95 (dd, J 7.9, 5.8 Hz, 1H), 2.43 (s, 3H), 2.25-1.78 (m, 3H), 1.44-0.91 (m, 5H), 0.89 (d, J 6.5 Hz, 3H).

### Intermediate 22

### [(S)-Cyano(trans-4-methylcyclohexyl)methyl]ammonium chloride

To a stirred solution of Intermediate 21 (6.6 g, 22.73 mmol) in dry methanol (130 mL) was added 4M hydrogen chloride in 1,4-dioxane (60 mL) dropwise over 2 minutes, whereupon an exotherm to 26° C. had occurred. The reaction mixture was cooled externally and 4M hydrogen chloride (60 mL) in 1,4-dioxane was added over 3 minutes. After 5 minutes, the flask was stoppered and the reaction mixture was stirred at ambient temperature for 2 h. The volatiles were concentrated in vacuo. Diethyl ether (100 mL) was added, then the mixture was sonicated and stirred for 15 minutes. The solids were filtered off and washed with diethyl ether (3×100 mL), then dried under a stream of nitrogen gas, to afford the title compound (4.10 g, 96%) as a white solid. δ_{H} (500 MHz, DMSO-d₆) 9.20 (s, 3H), 4.50 (d, J 5.5 Hz, 1H), 1.92-1.77 (m, 3H), 1.77-1.67 (m, 2H), 1.29 (ddp, J 11.4, 6.8, 3.4 Hz, 1H), 1.18-1.01 (m, 2H), 0.95-0.83 (m, 5H). HPLC-MS (method 1): [M+H]⁺ m/z 153, RT 0.46 minutes (100%). Chiral LC (method 22): RT 8.84 minutes (S, 93%).

### Intermediate 23

### [(S)-Carboxy(trans-4-methylcyclohexyl)methyl]ammonium chloride

A stirred solution of Intermediate 22 (4.05 g, 21.46 mmol) in a mixture of acetic acid (17 mL) and concentrated hydrochloric acid (85 mL) was heated to an external temperature of 130° C. (105° C. internal temperature). After 3 h, another portion of concentrated hydrochloric acid (25 mL) was added, followed by another portion (25 mL) after a further 2 h. The reaction mixture was heated for 1 h, then cooled. The precipitated solid was filtered and rinsed with tert-butyl methyl ether, then dried in vacuo, to afford the title compound (3.04 g, 68%) as a white solid. δ_{H} (500 MHz, DMSO-d₆) 8.35 (s, 3H), 3.69 (d, J 4.2 Hz, 1H), 1.82-1.65 (m, 4H), 1.64-1.54 (m, 1H), 1.32-1.18 (m, 2H), 1.15-1.02 (m, 1H), 0.93-0.80 (m, 5H). HPLC-MS (method 2): [M+H]+m/z 172, RT 0.63 minutes.

### Intermediate 24

### (2S)-2-(tert-Butoxycarbonylamino)-2-(trans-4-methylcyclohexyl)acetic acid

To a stirred suspension of Intermediate 23 (25.1 g, 120.8 mmol) in water (350 mL) was added sodium carbonate (55 g, 0.52 mol), then di-tert-butyl dicarbonate (39.6 g, 181 mmol) in 1,4-dioxane (500 mL). The reaction mixture was stirred for 4 h. The volatiles were removed in vacuo, then the suspension was cooled and 1N hydrochloric acid was carefully added to achieve a pH of 1. The mixture was extracted with ethyl acetate (3×250 mL). The organic layers were combined, washed in turn with water (200 mL) and brine (200 mL), then filtered through phase separating paper. The volatiles were evaporated. The resulting solid was triturated in heptane (500 mL), then filtered, washed with heptane (2×100 mL) and oven-dried, to give the title compound (28.8 g, 87%) as a white solid. δ_{H} (500 MHz, DMSO-d₆) 12.40 (s, 1H), 6.89 (d, J 8.5 Hz, 1H), 3.81-3.74 (m, 1H), 1.69-1.53 (m, 5H), 1.37 (s, 9H), 1.28-1.19 (m, 1H), 1.09 (dp, J 22.9, 12.6, 11.6 Hz, 2H), 0.91-0.76 (m, 5H). HPLC-MS (method 1): [M+H]+m/z 271, RT 3.34 minutes. Chiral SFC (method 23): RT 2.61 minutes (100%). [α]^{D}₂₀ 28.3° (c 3.202, chloroform).

### Intermediate 25 (Procedure A)

### tert-Butyl N-{(1S)-1-(trans-4-methylcyclohexyl)-2-oxo-2-[4-(tetrahydropyran-4-yl)-anilino]ethyl}carbamate

Intermediate 24 (250 mg, 0.92 mmol) was dissolved in DCM (6 mL), then HATU (433 mg, 1.11 mmol) and DIPEA (0.321 mL, 1.85 mmol) were added at r.t. After 5-10 minutes, 4-(tetrahydropyran-4-yl)aniline (172 mg, 0.97 mmol) was added. The reaction mixture was stirred at r.t. overnight, then diluted with DCM (15 mL) and quenched with water (20 mL). The aqueous phase was extracted once with DCM. The combined organic extracts were passed through a hydrophobic phase separator and dried under vacuum. The resulting orange residue was purified by column chromatography, using a gradient of isohexane/EtOAc (0-100%), then MeOH/EtOAc (0-30%), to yield the title compound (411 mg, 98%) as a pale brown solid. δ_{H} (300 MHz, CDCl₃) 7.75 (s, 1H), 7.53-7.41 (m, 2H), 7.18 (d, J 8.4 Hz, 2H), 5.06 (s, 1H), 4.16-4.02 (m, 2H), 3.95 (dd, J 8.5, 6.7 Hz, 1H), 3.59-3.42 (m, 2H), 2.81-2.64 (m, 1H), 1.91-1.63 (m, 11H), 1.45 (s, 9H), 1.39-1.19 (m, 1H), 1.19-0.91 (m, 2H), 0.87 (d, J 6.5 Hz, 3H). HPLC-MS (method 7): [M-^{t}Bu+H]⁺ m/z 375, RT 1.27 minutes.

### Intermediate 26 (Procedure B)

### {(1S)-1-(trans-4-Methylcyclohexyl)-2-oxo-2-[4-(tetrahydropyran-4-yl)anilino]ethyl}-ammonium chloride

Intermediate 25 (411 mg, 0.91 mmol) was dissolved in MeOH (8 mL) and DCM (4 mL). The mixture was treated with HCl in 1,4-dioxane (4N, 1.82 mL, 7.28 mmol) and stirred at r.t. overnight. The reaction was concentrated in vacuo to afford the crude title compound, which was utilised without further purification.

### Intermediate 27

### tert-Butyl N-(4-bromo-3,5-difluorophenyl)carbamate

4-Bromo-3,5-difluoroaniline (2.58 g, 11.32 mmol) was suspended in water (20 mL), and di-tert-butyl dicarbonate (3.08 g, 13.7 mmol) was added. The mixture was stirred vigorously for 72 h, then THF (10 mL) was added. After stirring at r.t. for 1.5 h, di-tert-butyl dicarbonate (1.65 g) was added portionwise. The mixture was stirred at r.t. for another 18 h, then concentrated in vacuo. The residue was purified by column chromatography, using a gradient of EtOAc/isohexane (0-20%), to yield the title compound (2.63 g, 75%) as a white crystalline solid. HPLC-MS (method 7): [M−^{t}Bu+H]⁺ m/z 252 and 254, RT 1.28 minutes.

### Intermediate 28

### [3,5-Difluoro-4-(tetrahydropyran-4-yl)phenyl]ammonium trifluoroacetate

In a capped vial, nickel chloride dimethoxyethane adduct (5.4 mg, 0.024 mmol) and 4,4′-di-tert-butyl-2,2′-dipyridyl (8 mg, 0.029 mmol) were suspended in anhydrous 1,2-dimethoxyethane (2 mL). Nitrogen gas was bubbled through the suspension, which was stirred for 10 minutes. In a second vial, Intermediate 27 (100 mg, 0.325 mmol) and {Ir[dF(CF₃)ppy]₂(dtbpy)}PF₆ (3.8 mg, 3.2 μmol) were dissolved in anhydrous 1,2-dimethoxyethane (2.4 mL) under nitrogen, then 4-bromotetrahydropyran (58 μL, 0.33 mmol), 2,6-lutidine (76 μL, 0.646 mmol) and tris(trimethylsilyl)silane (100 μL, 0.33 mmol) were added. A portion of the nickel chloride/dipyridyl solution (0.1 mL) was added to the second vial, and the vessel was sparged with nitrogen for 10 minutes. The tube was sealed and the mixture was stirred at ambient temperature, whilst undergoing irradiation with a blue LED (450 nm) for 2 h. The residue was purified by column chromatography, using a gradient of EtOAc/isohexane (0-100%). The resulting crude tert-butyl N-[3,5-difluoro-4-(tetrahydropyran-4-yl)phenyl]carbamate, a brown solid (11 mg), was dissolved in DCM (1.5 mL), treated with TFA (0.2 mL) and stirred at r.t. for 18 h. The reaction mixture was concentrated in vacuo, and azeotroped with toluene, to yield the crude title compound (11 mg), which was utilised without further purification. HPLC-MS (method 7): [M+H]⁺ m/z 214, RT 0.81 minutes.

### Intermediate 29

### Ethyl 2-(4-nitrophenyl)acetate

To a solution of 4-nitrophenylacetic acid (10.0 g, 55.2 mmol) in EtOH (80 mL) was added thionyl chloride (0.80 mL, 11.0 mmol). The reaction mixture was stirred at r.t. for 16 h, then concentrated in vacuo. The residue was dissolved in DCM (600 mL), then washed with water (600 mL) and saturated aqueous sodium hydrogen carbonate solution (600 mL). The organic layer was separated, then dried over anhydrous sodium sulfate and concentrated in vacuo, to afford the title compound (11.0 g, 95%) as an off-white solid. δ_{H} (400 MHz, DMSO-d₆) 8.20 (d, J 8.31 Hz, 2H), 7.57 (d, J 8.31 Hz, 2H), 4.10 (q, J 6.85 Hz, 2H), 3.88 (s, 2H), 1.19 (t, J 7.09 Hz, 3H).

### Intermediate 30

### Ethyl 4-(4-nitrophenyl)tetrahydro-2H-pyran-4-carboxylate

To a solution of Intermediate 29 (11.0 g, 52.6 mmol) in DMF (100 mL) was added NaH (2.31 g, 57.8 mmol) at 0° C. The reaction mixture was stirred for 30 minutes, then 1-bromo-2-(2-bromoethoxy)ethane (18.3 g, 78.9 mmol) was added dropwise. The reaction mixture was heated at 80° C. for 16 h, then cooled to room temperature, quenched with water (1000 mL) and extracted with EtOAc (3×600 mL). The organic layer was separated, dried over anhydrous sodium sulfate and concentrated in vacuo. The crude residue was purified by column chromatography (silica, 100-200 mesh, 10% EtOAc in hexanes) to afford the title compound (6.0 g, 41%) as an off-white solid. δ_{H} (400 MHz, DMSO-d₆) 8.23 (d, J 8.80 Hz, 2H), 7.67 (d, J 8.80 Hz, 2H), 4.11 (q, J 6.85 Hz, 2H), 3.78-3.87 (m, 2H), 3.42-3.49 (m, 2H), 2.40-2.43 (m, 2H), 1.89-1.99 (m, 2H), 1.11 (t, J 7.09 Hz, 3H).

### Intermediate 31

### 4-(4-Nitrophenyl)tetrahydro-2H-pyran-4-carboxylic acid

To a solution of Intermediate 30 (0.70 g, 2.51 mmol) in THF (7 mL) and water (3 mL) was added LiOH.H₂O (0.42 g, 10.0 mmol). The reaction mixture was stirred at r.t. for 16 h, then concentrated in vacuo. The residue was diluted with water (15 mL), then acidified with 1M HCl to pH 4 and extracted with EtOAc (2×25 mL). The organic layer was separated, then dried over anhydrous sodium sulfate and concentrated in vacuo, to afford the title compound (0.60 g, 83%) as a white solid. δ_{H} (400 MHz, DMSO-d₆) 13.04 (br s, 1H), 8.23 (d, J 8.80 Hz, 2H), 7.69 (d, J 8.80 Hz, 2H), 3.80-3.86 (m, 2H), 3.46-3.51 (m, 2H), 2.40 (d, J 13.21 Hz, 2H), 1.84-1.93 (m, 2H).

### Intermediate 32

### N,N-Dimethyl-4-(4-nitrophenyl)tetrahydro-2H-pyran-4-carboxamide

To a solution of Intermediate 31 (0.60 g, 2.39 mmol) and dimethylamine (2M solution in THF, 3.58 mL, 7.16 mmol) in DCM (6 mL) were added DIPEA (0.83 mL, 4.78 mmol) and HATU (50%, 2.18 g, 2.87 mmol). The reaction mixture was stirred at r.t. for 4 h, then quenched with water (10 mL) and extracted with EtOAc (3×10 mL). The organic layer was separated, dried over anhydrous sodium sulfate and concentrated in vacuo. The crude residue was purified by column chromatography (70% EtOAc in hexanes) to afford the title compound (0.42 g, 63%) as a white solid. δ_{H} (400 MHz, DMSO-d₆) 8.24 (d, J 8.80 Hz, 2H), 7.53 (d, J 8.80 Hz, 2H), 3.76-3.79 (m, 2H), 3.53-3.66 (m, 2H), 2.69 (s, 6H), 2.20 (d, J 13.69 Hz, 2H), 1.88-2.00 (m, 2H). HPLC-MS (method 18): [M+H]⁺ m/z 279, RT 1.64 minutes.

### Intermediate 33

### 4-(4-Aminophenyl)-N,N-dimethyltetrahydro-2H-pyran-4-carboxamide

To a solution of Intermediate 32 (0.42 g, 1.51 mmol) in MeOH (6 mL) was added SnCl₂.2H₂O (1.02 g, 4.53 mmol). The reaction mixture was stirred at r.t. for 16 h, then concentrated in vacuo. The residue was washed with 2% MeOH in DCM (3×25 mL), then decanted and dried in vacuo, to afford the title compound (0.50 g, 87%) as an off-white solid. δ_{H} (400 MHz, DMSO-d₆) 6.88 (d, J 8.80 Hz, 2H), 6.57 (d, J 8.80 Hz, 2H), 4.12 (br s, 2H), 3.68-3.75 (m, 2H), 3.54 (t, J 10.52 Hz, 2H), 3.17 (s, 6H), 2.11 (d, J 13.69 Hz, 2H), 1.76-1.85 (m, 2H). HPLC-MS (method 18): [M+H]⁺ m/z 249, RT 1.38 minutes.

### Intermediate 34

### tert-Butyl N-{(1S)-2-[3-fluoro-4-(tetrahydropyran-4-yl)anilino]-1-(trans-4-methyl-cyclohexyl)-2-oxoethyl}carbamate

Intermediate 24 (100 mg, 0.37 mmol) was dissolved in DCM (3 mL), then HATU (173 mg, 0.44 mmol) and DIPEA (128 μL, 0.74 mmol) were added at r.t. After 5-10 minutes, Intermediate 17 (76 mg, 0.39 mmol) was added as a solution in DCM (2 mL). The reaction mixture was stirred at r.t. overnight, then diluted with DCM (5 mL) and quenched with water (5 mL). The aqueous phase was extracted three times with DCM. The combined organic layers were passed through a hydrophobic phase separator and concentrated in vacuo. The crude material was purified by column chromatography, using a gradient of EtOAc/DCM (0-50%), then MeOH/DCM (0-10%), to yield the title compound (191 mg, 97%) as an off-white solid. δ_{H} (400 MHz, CDCl₃) 7.96 (s, 1H), 7.46 (dd, J 12.2, 2.1 Hz, 1H), 7.14 (t, J 8.1 Hz, 1H), 7.09 (dd, J 8.4, 2.1 Hz, 1H), 5.05 (s, 1H), 4.09-4.03 (m, 2H), 3.94 (dd, J 8.5, 6.8 Hz, 1H), 3.54 (td, J 11.7, 2.3 Hz, 3H), 3.06 (tt, J 11.8, 3.9 Hz, 1H), 1.88-1.66 (m, 8H), 1.45 (s, 10H), 1.37-1.19 (m, 3H), 1.19-0.89 (m, 3H), 0.87 (d, J 6.5 Hz, 3H). HPLC-MS (method 7): [M−^{t}Bu+H]⁺ m/z 393, RT 1.34 minutes.

### Intermediate 35

### (2S)-2-Amino-N-[3-fluoro-4-(tetrahydropyran-4-yl)phenyl]-2-(trans-4-methyl-cyclohexyl)acetamide trifluoroacetate Salt

Intermediate 34 (191 mg, 0.38 mmol) was dissolved in DCM (4 mL) under an atmosphere of nitrogen, then cooled to 0° C. (ice bath) and treated with TFA (0.5 mL, 7 mmol). The reaction mixture was stirred for 18 h, then concentrated in vacuo, to afford the crude title compound, which was utilised without further purification.

### Intermediate 36

### [4-(4-Aminophenyl)tetrahydropyran-4-yl](3,3-difluoroazetidin-1-yl)methanone

4-(4-Aminophenyl)tetrahydropyran-4-carboxylic acid (500 mg, 2.26 mmol), 3,3-difluoroazetidine hydrochloride (324 mg, 2.37 mmol) and HATU (930 mg, 2.37 mmol) were added to a round-bottomed flask, followed by DCM (30 mL) and DIPEA (0.83 mL, 4.8 mmol). The reaction mixture was stirred at r.t. for 3 h, then quenched with saturated aqueous sodium hydrogen carbonate solution (20 mL) and extracted with DCM (3×15 mL). The organic extracts were combined, filtered through a hydrophobic frit and concentrated in vacuo to afford the title compound (800 mg crude, 100%) as an off-white solid, which was utilised without further purification. δ_{H} (300 MHz, DMSO-d₆) 6.99-6.85 (m, 2H), 6.63-6.50 (m, 2H), 5.13 (s, 2H), 4.08 (s, 4H), 3.70 (dt, J 11.6, 4.0 Hz, 2H), 3.53 (td, J 11.6, 2.3 Hz, 2H), 2.14-2.09 (m, 2H), 1.80 (ddd, J 13.9, 10.0, 4.1 Hz, 2H). LCMS (method 8): MH+ m/z 297.0, RT 0.86 minutes.

### Intermediate 37

### Benzyl N-[(1S)-1-cyclohexyl-2-{4-[4-(3,3-difluoroazetidine-1-carbonyl)tetrahydropyran-4-yl]anilino}-2-oxoethyl]carbamate

Intermediate 36 (500 mg, 1.68 mmol), (2S)-2-cyclohexyl-2-(phenoxycarbonyl-amino)acetic acid (516 mg, 1.77 mmol) and HATU (694 mg, 1.77 mmol) were added to a round-bottomed flask, followed by DCM (20 mL). The mixture was stirred at r.t., then DIPEA (0.62 mL, 3.6 mmol) was added. The mixture was stirred at r.t. for 18 h, then concentrated in vacuo and purified by column chromatography (silica, 10-50% gradient of EtOAc in DCM). The combined fractions were concentrated in vacuo to afford the title compound (430 mg, 45%) as a white solid. δ_{H} (300 MHz, DMSO-d₆) 10.13 (s, 1H), 7.65 (d, J 8.5 Hz, 2H), 7.49 (d, J 8.4 Hz, 1H), 7.41-7.28 (m, 4H), 7.27-7.19 (m, 2H), 5.03 (s, 2H), 4.47-4.00 (br s, 4H), 4.10-3.86 (m, 2H), 3.72 (d, J 11.5 Hz, 2H), 3.56 (t, J 10.6 Hz, 2H), 2.18 (d, J 13.4 Hz, 2H), 1.96-1.42 (m, 7H), 1.28-0.94 (m, 6H). LCMS (method 8): MH⁺ m/z 570.4, RT 2.21 minutes.

### Intermediate 38

### (2S)-2-Amino-2-cyclohexyl-N-{4-[4-(3,3-difluoroazetidine-1-carbonyl)tetrahydropyran-4-yl]phenyl}acetamide

Intermediate 37 (405 mg, 0.71 mmol) was dissolved in ethanol (30 mL) and ethyl acetate (10 mL). Pd/C (10% w/w, 80 mg) was added. The mixture was degassed and placed under a hydrogen atmosphere (balloon), then stirred for 18 h. The mixture was degassed and filtered through a celite pad, then washed with ethanol (2×5 mL) and concentrated in vacuo, to afford the title compound (308 mg, 99%) as a white solid. δ_{H} (300 MHz, DMSO-d₆) 10.00 (br s, 1H), 7.77-7.59 (m, 2H), 7.28-7.17 (m, 2H), 4.35 (t, J 5.1 Hz, 1H), 4.12 (br s, 4H), 3.72 (dt, J 11.4, 4.0 Hz, 2H), 3.56 (t, J 10.5 Hz, 2H), 3.49-3.38 (m, 1H), 3.10 (d, J 5.7 Hz, 1H), 2.18 (d, J 13.4 Hz, 2H), 1.87 (ddd, J 13.6, 9.9, 3.9 Hz, 2H), 1.79-1.46 (m, 5H), 1.12 (m, 6H). LCMS (method 8): MH⁺ m/z 436.0, RT 1.57 minutes.

### Example 1

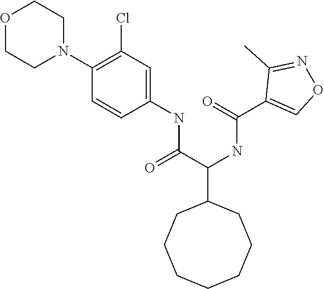 [SMILES: Cc1nocc1C(=O)NC(C(=O)Nc1ccc(N2CCOCC2)c(Cl)c1)C1CCCCCCC1]

### N-{2-[3-Chloro-4-(morpholin-4-yl)anilino]-1-cyclooctyl-2-oxoethyl}-3-methylisoxazole-4-carboxamide

A solution of 3-chloro-4-(morpholin-4-yl)aniline (37 mg, 0.17 mmol) in DMF (700 μL) was added to Intermediate 5 (50 mg, 0.17 mmol), followed by DIPEA (40 μL, 0.187 mmol) and HATU (80 mg, 0.187 mmol). The resulting suspension was stirred at r.t. for 60 h in a sealed tube. The reaction mixture was purified by preparative HPLC (method 20) to yield the title compound (30 mg, 36%) as a white solid. δ_{H} (400 MHz, DMSO-d₆) 10.31 (s, 1H), 9.52-9.28 (m, 1H), 8.51 (d, J 8.6 Hz, 1H), 7.84 (d, J 2.4 Hz, 1H), 7.49 (dd, J 8.7, 2.5 Hz, 1H), 7.15 (d, J 8.8 Hz, 1H), 4.43 (t, J 8.7 Hz, 1H), 3.73 (t, J 4.5 Hz, 4H), 2.92 (t, J 4.7 Hz, 4H), 2.37 (s, 3H), 1.79-1.28 (m, 15H). uPLC-MS (method 15): [M+H]⁺ m/z 489 and 491, RT 2.99 minutes.

### Example 2

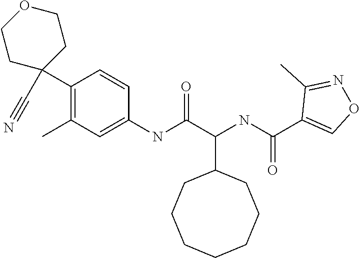 [SMILES: Cc1cc(NC(=O)C(NC(=O)c2conc2C)C2CCCCCCC2)ccc1C1(C#N)CCOCC1]

### N-[4-(4-Cyanotetrahydropyran-4-yl)-3-methylphenyl]-2-cyclooctyl-2-[(3-methylisoxazol-4-yl)formamido]acetamide

A sealed tube was charged with EDC.HCl (81.4 mg, 0.42 mmol) and Intermediate 5 (125 mg, 0.42 mmol) in DCM (2 mL). The reaction mixture was stirred for 0.5 h at 20° C. The solvent was removed under a stream of nitrogen and Intermediate 10 (62.3 mg, 0.29 mmol) in THF (2 mL) was added, followed by acetic acid (0.25 mL, 4.33 mmol). The reaction mixture was sealed and heated at 60° C. for 1 h. After cooling, the reaction mixture was quenched with saturated aqueous sodium hydrogen carbonate solution (10 mL). The aqueous layer was extracted with EtOAc (2×20 mL). The combined organic extracts were washed with saturated aqueous sodium hydrogen carbonate solution (10 mL) and dried over sodium sulfate, then filtered and concentrated in vacuo. The resulting orange oil was purified by flash column chromatography, using a gradient of EtOAc/heptane (0-65%), to afford, after freeze-drying, the title compound (94 mg, 67%) as a cream-coloured solid. δ_{H} (500 MHz, DMSO-d₆) 10.27 (s, 1H), 9.44 (s, 1H), 8.48 (d, J 8.6 Hz, 1H), 7.59-7.54 (m, 1H), 7.53 (d, J 2.1 Hz, 1H), 7.29 (d, J 8.8 Hz, 1H), 4.51-4.44 (m, 1H), 4.04-3.96 (m, 2H), 3.77-3.68 (m, 2H), 2.54 (s, 3H), 2.38 (s, 3H), 2.29-2.22 (m, 2H), 2.15-2.06 (m, 1H), 2.03-1.93 (m, 2H), 1.73-1.62 (m, 3H), 1.61-1.45 (m, 7H), 1.45-1.36 (m, 4H). uPLC-MS (method 1): [M+H]⁺ m/z 493, RT 3.74 minutes.

### Example 3 (Procedure C)

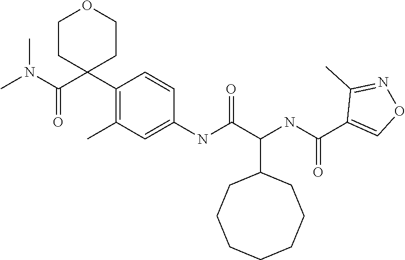 [SMILES: Cc1cc(NC(=O)C(NC(=O)c2conc2C)C2CCCCCCC2)ccc1C1(C(=O)N(C)C)CCOCC1]

### 4-(4-{2-Cyclooctyl-2-[(3-methylisoxazol-4-yl)formamido]acetamido}-2-methylphenyl)-N,N-dimethyltetrahydropyran-4-carboxamide

A sealed tube was charged with EDC.HCl (52 mg, 0.27 mmol) and Intermediate 5 (80 mg, 0.27 mmol) in DCM (1 mL). The reaction mixture was stirred for 0.5 h at 20° C. The solvent was removed using a flow of nitrogen and Intermediate 14 (46.9 mg, 0.18 mmol) in THF (1 mL) was added, followed by acetic acid (0.15 mL, 2.68 mmol). The reaction mixture was sealed and heated at 60° C. for 1 h. After cooling, the reaction mixture was quenched with saturated aqueous sodium hydrogen carbonate solution (10 mL). The aqueous layer was extracted with EtOAc (2×20 mL). The combined organic extracts were washed with saturated aqueous sodium hydrogen carbonate solution (10 mL) and dried over sodium sulfate, then filtered and concentrated in vacuo. The resulting orange oil was separated by flash column chromatography, using a gradient of EtOAc/heptane (0-100%), followed by preparative HPLC (method 13), to afford, after freeze-drying, the title compound (44 mg, 44%) as a white solid. δ_{H} (500 MHz, CD₃OD) 9.14 (s, 1H), 7.55 (dd, J 8.7, 2.4 Hz, 1H), 7.48 (d, J 8.7 Hz, 1H), 7.41 (d, J 2.2 Hz, 1H), 4.51 (d, J 8.4 Hz, 1H), 4.04-3.88 (m, 2H), 3.87-3.79 (m, 2H), 2.94 (s, 3H), 2.57 (s, 3H), 2.45 (s, 3H), 2.38-2.30 (m, 2H), 2.27-2.20 (m, 4H), 2.21-1.96 (m, 2H), 1.85-1.74 (m, 3H), 1.74-1.63 (m, 4H), 1.62-1.49 (m, 7H). uPLC-MS (method 1): [M+H]⁺ m/z 439, RT 3.45 minutes.

### Examples 4 to 19

The title compounds were prepared according to Procedure C from Intermediate 5 and the appropriate aniline or heteroaryl amine.

The aniline or heteroaryl amine starting materials for Examples 4 to 18 are commercially available. The aniline starting material for Example 19 is Intermediate 33.

| | | | | | LCMS |
|---|---|---|---|---|---|
| | | | LCMS | LCMS | RT |
| Ex. | Structure | Name | Method | Mass | (min) |
| 4 | 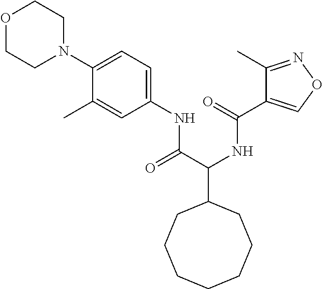 [SMILES: Cc1cc(NC(=O)C(NC(=O)c2conc2C)C2CCCCCCC2)ccc1N1CCOCC1] | N-{1-Cyclooctyl-2-[3- methyl-4-(morpholin- 4-yl)anilino]-2-oxo- ethyl}-3-methyl- isoxazole-4- carboxamide | 15 | 468.9 | 2.94 |
| 5 | 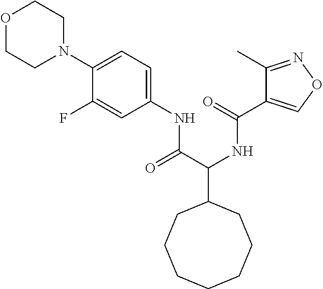 [SMILES: Cc1nocc1C(=O)NC(C(=O)Nc1ccc(N2CCOCC2)c(F)c1)C1CCCCCCC1] | N-{1-Cyclooctyl-2-[3- fluoro-4-(morpholin-4- yl)anilino]-2-oxo- ethyl}-3-methyl- isoxazole-4- carboxamide | 15 | 472.9 | 2.83 |
| 6 | 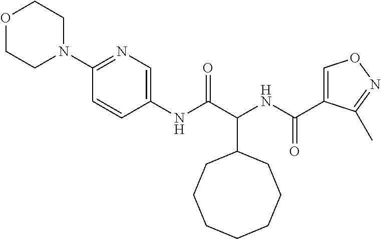 [SMILES: Cc1nocc1C(=O)NC(C(=O)Nc1ccc(N2CCOCC2)nc1)C1CCCCCCC1] | N-(1-Cyclooctyl-2-{[6- (morpholin-4-yl)- pyridin-3-yl]amino}-2- oxoethyl)-3-methyl- isoxazole-4- carboxamide | 14 | 456.2 | 1.86 |
| 7 | 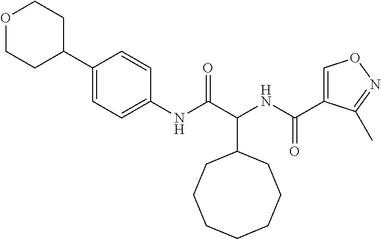 [SMILES: Cc1nocc1C(=O)NC(C(=O)Nc1ccc(C2CCOCC2)cc1)C1CCCCCCC1] | N-{1-Cyclooctyl-2- oxo-2-[4-(tetrahydro- pyran-4-yl)anilino]- ethyl}-3-methyl- isoxazole-4- carboxamide | 14 | 454.3 | 2.44 |
| 8 | 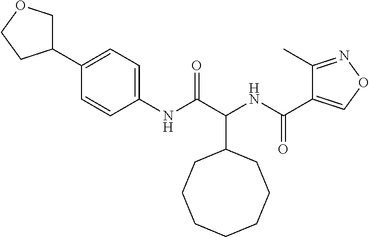 [SMILES: Cc1nocc1C(=O)NC(C(=O)Nc1ccc(C2CCOC2)cc1)C1CCCCCCC1] | N-{1-Cyclooctyl-2- oxo-2-[4-(tetrahydro- furan-3-yl)anilino]- ethyl}-3-methyl- isoxazole-4- carboxamide | 8 | 440.0 | 2.51 |
| 9 | 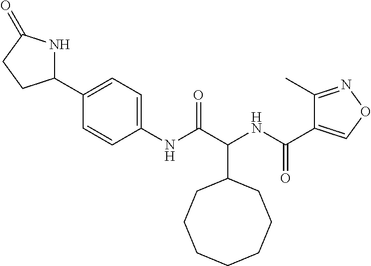 [SMILES: Cc1nocc1C(=O)NC(C(=O)Nc1ccc(C2CCC(=O)N2)cc1)C1CCCCCCC1] | N-{1-Cyclooctyl-2- oxo-2-[4-(5-oxo- pyrrolidin-2-yl)- anilino]ethyl}-3- methylisoxazole-4- carboxamide | 8 | 453.0 | 2.19 |
| 10 | 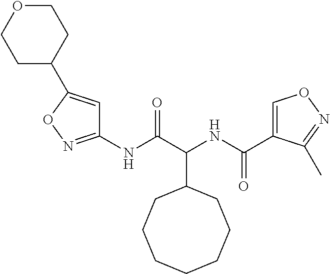 [SMILES: Cc1nocc1C(=O)NC(C(=O)Nc1cc(C2CCOCC2)on1)C1CCCCCCC1] | N-(1-Cyclooctyl-2-oxo- 2-{[5-(tetrahydropyran- 4-yl)isoxazol-3-yl]- amino}ethyl)-3- methylisoxazole-4- carboxamide | 14 | 445.0 | 2.13 |
| 11 | 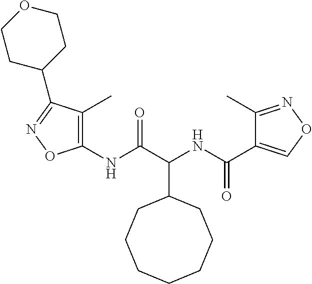 [SMILES: Cc1nocc1C(=O)NC(C(=O)Nc1onc(C2CCOCC2)c1C)C1CCCCCCC1] | N-(1-Cyclooctyl-2-{[4- methyl-3-(tetrahydro- pyran-4-yl)isoxazol-5- yl]amino}-2-oxoethyl)- 3-methylisoxazole-4- carboxamide | 13 | 459.0 | 2.64 |
| 12 | 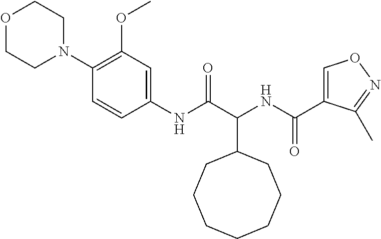 [SMILES: COc1cc(NC(=O)C(NC(=O)c2conc2C)C2CCCCCCC2)ccc1N1CCOCC1] | N-{1-Cyclooctyl-2-[3- methoxy-4-(morpholin- 4-yl)anilino]-2-oxo- ethyl}-3-methyl- isoxazole-4- carboxamide | 14 | 485.3 | 2.09 |
| 13 | 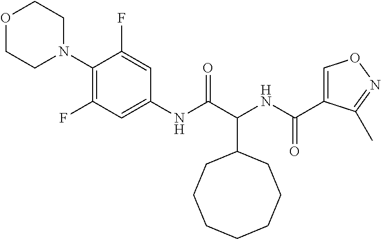 [SMILES: Cc1nocc1C(=O)NC(C(=O)Nc1cc(F)c(N2CCOCC2)c(F)c1)C1CCCCCCC1] | N-{1-Cyclooctyl-2- [3,5-difluoro-4- (morpholin-4-yl)- anilino]-2-oxoethyl}-3- methylisoxazole-4- carboxamide | 14 | 491.3 | 2.55 |
| 14 | 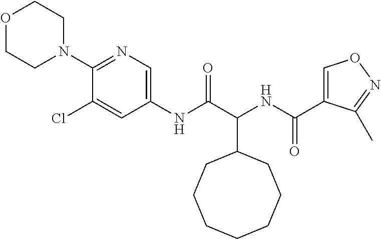 [SMILES: Cc1nocc1C(=O)NC(C(=O)Nc1cnc(N2CCOCC2)c(Cl)c1)C1CCCCCCC1] | N-(2-{[5-Chloro-6- (morpholin-4-yl)- pyridin-3-yl]amino}-1- cyclooctyl-2-oxoethyl)- 3-methylisoxazole-4- carboxamide | 14 | 490.2 & 492.2 | 2.42 |
| 15 | 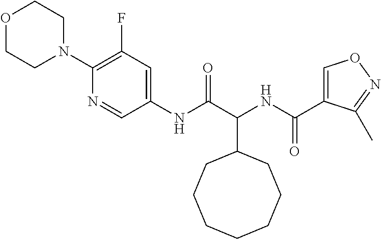 [SMILES: Cc1nocc1C(=O)NC(C(=O)Nc1cnc(N2CCOCC2)c(F)c1)C1CCCCCCC1] | N-(1-Cyclooctyl-2-{[5- fluoro-6-(morpholin-4- yl)pyridin-3-yl]- amino}-2-oxoethyl)-3- methylisoxazole-4- carboxamide | 14 | 474.3 | 2.31 |
| 16 | 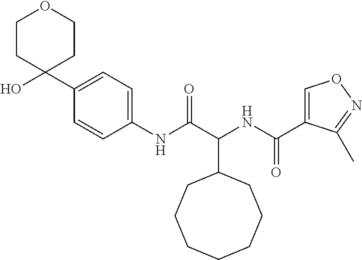 [SMILES: Cc1nocc1C(=O)NC(C(=O)Nc1ccc(C2(O)CCOCC2)cc1)C1CCCCCCC1] | N-{1-Cyclooctyl-2-[4- (4-hydroxytetrahydro- pyran-4-yl)anilino]-2- oxoethyl}-3-methyl- isoxazole-4- carboxamide | 14 | 470.3 | 2.11 |
| 17 | 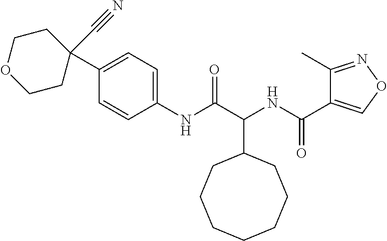 [SMILES: Cc1nocc1C(=O)NC(C(=O)Nc1ccc(C2(C#N)CCOCC2)cc1)C1CCCCCCC1] | N-{2-[4-(4-Cyano- tetrahydropyran-4-yl)- anilino]-1-cyclooctyl- 2-oxoethyl}-3-methyl- isoxazole-4- carboxamide | 12 | 479.2 | 2.79 |
| 18 | 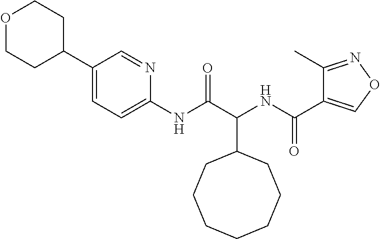 [SMILES: Cc1nocc1C(=O)NC(C(=O)Nc1ccc(C2CCOCC2)cn1)C1CCCCCCC1] | N-(1-Cyclooctyl-2-oxo- 2-{[5-(tetrahydropyran- 4-yl)pyridin-2-yl]- amino}ethyl)-3- methylisoxazole-4- carboxamide | 12 | 455.2 | 2.75 |
| 19 | 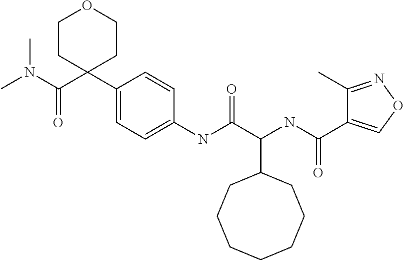 [SMILES: Cc1nocc1C(=O)NC(C(=O)Nc1ccc(C2(C(=O)N(C)C)CCOCC2)cc1)C1CCCCCCC1] | 4-(4-{2-Cyclooctyl-2- [(3-methylisoxazol-4- yl)formamido]- acetamido}phenyl)- N,N-dimethyl- tetrahydropyran-4- carboxamide | 24 | 525.0 | 2.76 |

Selected ¹H NMR data

Example 4: δ_{H} (400 MHz, CD₃OD) 9.14 (s, 1H), 7.40 (s, 1H), 7.45-7.27 (m, 1H), 7.04 (d, J 8.3 Hz, 1H), 3.93-3.71 (m, 4H), 2.88 (m, 4H), 2.46 (d, J 0.6 Hz, 3H), 2.32 (s, 3H), 2.19 (m, 1H), 1.85-1.44 (m, 15H).

Example 5: δ_{H} (400 MHz, DMSO-d₆) 10.31 (s, 1H), 9.44 (s, 1H), 8.54 (d, J 8.6 Hz, 1H), 7.59 (dd, J 14.9, 2.3 Hz, 1H), 7.35-7.13 (m, 1H), 7.01 (dd, J 9.9, 8.8 Hz, 1H), 4.44 (t, J 8.7 Hz, 1H), 3.77-3.69 (m, 4H), 2.98-2.91 (m, 4H), 2.38 (s, 3H), 2.13-2.04 (m, 1H), 1.72-1.31 (m, 14H).

### Example 20

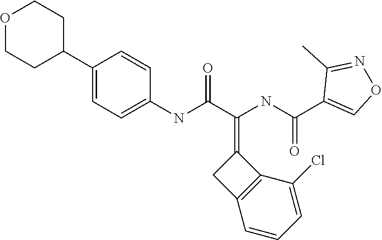 [SMILES: Cc1nocc1C(=O)N/C(C(=O)Nc1ccc(C2CCOCC2)cc1)=C1/Cc2cccc(Cl)c21]

### 2-[(7Z)-5-Chlorobicyclo[4.2.0]octa-1,3,5-trien-7-ylidene]-2-[(3-methylisoxazol-4-yl)-formamido]-N-[4-(tetrahydropyran-4-yl)phenyl]acetamide

Acetic acid (121 μL, 2.11 mmol) was added to a stirred solution of Intermediate 15 (65 mg, 0.21 mmol) and 4-(tetrahydro-2H-pyran-4-yl)aniline (38 mg, 0.21 mmol) in anhydrous THF (1.5 mL). The vessel was purged with nitrogen, sealed and stirred at 60° C. for 18 h. Upon cooling to room temperature, the reaction mixture was concentrated in vacuo. The residue was purified by preparative HPLC (method 15) to afford, after freeze-drying, the title compound (14.1 mg, 13%) as an off-white solid. δ_{H} (500 MHz, DMSO-d₆) 10.08 (s, 1H), 9.99 (s, 1H), 9.43 (s, 1H), 7.63 (d, J 8.5 Hz, 2H), 7.42-7.35 (m, 1H), 7.31 (d, J 8.0 Hz, 1H), 7.29 (d, J 7.1 Hz, 1H), 7.21 (d, J 8.6 Hz, 2H), 4.04-3.86 (m, 4H), 3.43 (td, J 11.3, 3.1 Hz, 2H), 2.80-2.67 (m, 1H), 2.40 (s, 3H), 1.82-1.57 (m, 4H). uPLC-MS (method 1): [M+H]⁺ m/z 476 and 478, RT 3.31 minutes.

### Example 21 (Procedure D)

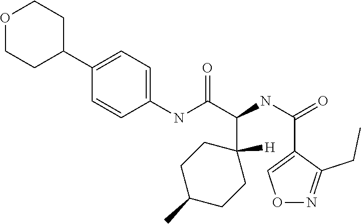 [SMILES: CCc1nocc1C(=O)N[C@H](C(=O)Nc1ccc(C2CCOCC2)cc1)[C@H]1CC[C@H](C)CC1]

### 3-Ethyl-N-{(1S)-1-(trans-4-methylcyclohexyl)-2-oxo-2-[4-(tetrahydropyran-4-yl)anilino]-ethyl}isoxazole-4-carboxamide

3-Ethylisoxazole-4-carboxylic acid (40.51 mg, 0.29 mmol), HATU (135.0 mg, 0.344 mmol) and DIPEA (200 μL, 1.15 mmol) were stirred in DCM (1 mL) for 15 minutes at r.t. Intermediate 26 (100 mg, 0.29 mmol) was added in one portion as a solution in DCM (3 mL). The reaction mixture was stirred at room temperature overnight, then concentrated under a stream of nitrogen. The residue was purified by column chromatography, using a gradient of EtOAc/isohexane (0-100%), then MeOH/EtOAc (0-20%), to yield the title compound as a white solid (98 mg, 67%). δ_{H} (400 MHz, DMSO-d₆) 10.34 (s, 1H), 9.41 (s, 1H), 8.50 (d, J 8.2 Hz, 1H), 7.57 (dd, J 13.2, 1.8 Hz, 1H), 7.32-7.24 (m, 2H), 4.36 (t, J 8.4 Hz, 1H), 3.98-3.86 (m, 2H), 3.44 (td, J 11.6, 2.3 Hz, 2H), 2.98 (tt, J 11.7, 3.9 Hz, 1H), 2.83 (q, J 7.6 Hz, 2H), 1.88-1.77 (m, 1H), 1.77-1.49 (m, 8H), 1.35-1.25 (m, 1H), 1.25-1.18 (m, 1H), 1.20-1.12 (m, 4H), 1.10-0.95 (m, 1H), 0.94-0.76 (m, 4H). HPLC-MS (method 8): [M+H]⁺ m/z 454, RT 2.46 minutes.

### Examples 22 to 30

The title compounds were prepared by a three-step procedure comprising:

(i) reacting Intermediate 7 or Intermediate 24 and the appropriate aniline or heteroaryl amine according to Procedure A;

(ii) deprotection of the material thereby obtained according to procedure B; and

(iii) reacting the material thereby obtained with a commercially available acid according to procedure D.

The aniline starting materials in step (i) for Examples 22 and 30 are Intermediates 17 and 28 respectively. The corresponding starting materials for Examples 23-29 are commercially available anilines or heteroaryl amines.

| | | | | | LCMS |
|---|---|---|---|---|---|
| | | | LCMS | LCMS | RT |
| Ex. | Structure | Name | Method | Mass | (min) |
| 22 | 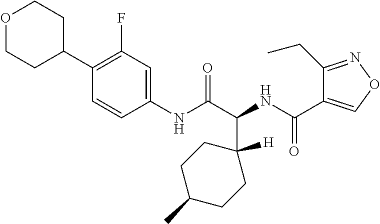 [SMILES: CCc1nocc1C(=O)N[C@H](C(=O)Nc1ccc(C2CCOCC2)c(F)c1)[C@H]1CC[C@H](C)CC1] | 3-Ethyl-N-{(1S)-2-[3- fluoro-4-(tetrahydro- pyran-4-yl)anilino]-1- (trans-4-methylcyclo- hexyl)-2-oxoethyl}- isoxazole-4- carboxamide | 8 | 472.0 | 2.66 |
| 23 | 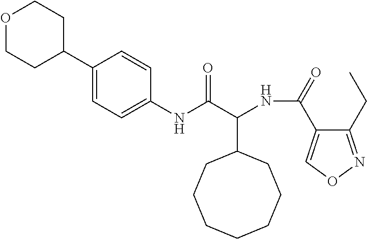 [SMILES: CCc1nocc1C(=O)NC(C(=O)Nc1ccc(C2CCOCC2)cc1)C1CCCCCCC1] | N-{1-Cyclooctyl-2- oxo-2-[4-(tetrahydro- pyran-4-yl)anilino]- ethyl}-3-ethyl- isoxazole-4- carboxamide | 8 | 468.0 | 2.78 |
| 24 | 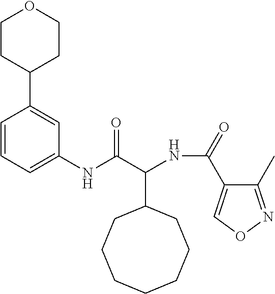 [SMILES: Cc1nocc1C(=O)NC(C(=O)Nc1cccc(C2CCOCC2)c1)C1CCCCCCC1] | N-{1-Cyclooctyl-2- oxo-2-[3-(tetrahydro- pyran-4-yl)anilino]- ethyl}-3-methyl- isoxazole-4- carboxamide | 8 | 454.0 | 2.65 |
| 25 | 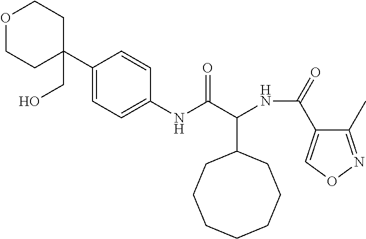 [SMILES: Cc1nocc1C(=O)NC(C(=O)Nc1ccc(C2(CO)CCOCC2)cc1)C1CCCCCCC1] | N-(1-Cyclooctyl-2-{4- [4-(hydroxymethyl)- tetrahydropyran-4-yl]- anilino}-2-oxoethyl)-3- methylisoxazole-4- carboxamide | 8 | 484.0 | 2.17 |
| 26 | 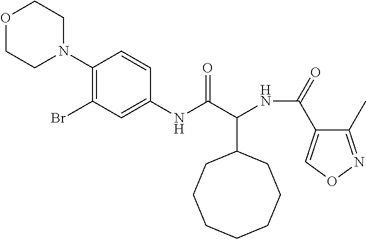 [SMILES: Cc1nocc1C(=O)NC(C(=O)Nc1ccc(N2CCOCC2)c(Br)c1)C1CCCCCCC1] | N-{2-[3-Bromo-4- (morpholin-4-yl)- anilino]-1-cyclooctyl- 2-oxoethyl}-3-methyl- isoxazole-4- carboxamide | 8 | 535.0 & 537.0 | 2.81 |
| 27 | 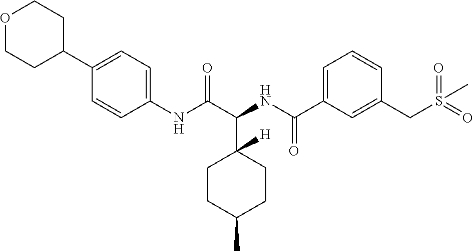 [SMILES: CS(=O)(=O)Cc1cccc(C(=O)N[C@H](C(=O)Nc2ccc(C3CCOCC3)cc2)[C@H]2CC[C@H](C)CC2)c1] | N-{(1S)-1-(4-Methyl- cyclohexyl)-2-oxo-2- [4-(tetrahydropyran-4- yl)anilino]ethyl}-3- (methylsulfonyl- methyl)benzamide | 10 | 527.3 | 1.96 |
| 28 | 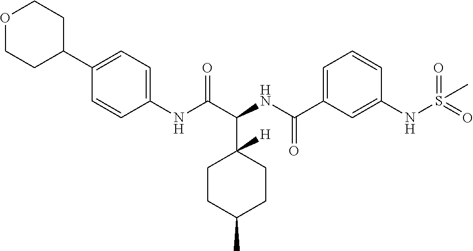 [SMILES: CS(=O)(=O)Nc1cccc(C(=O)N[C@H](C(=O)Nc2ccc(C3CCOCC3)cc2)[C@H]2CC[C@H](C)CC2)c1] | 3-(Methane- sulfonamido)-N-{(1S)- 1-(trans-4-methyl- cyclohexyl)-2-oxo-2- [4-(tetrahydropyran-4- yl)anilino]ethyl}- benzamide | 10 | 528.2 | 1.96 |
| 29 | 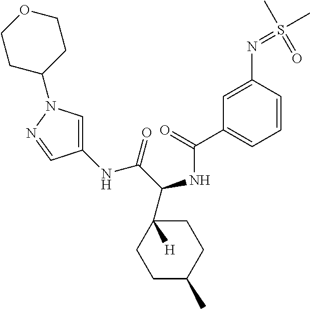 | 3-{[Dimethyl(oxo)-λ⁶- sulfanylidene]amino}- N-[(1S)-1-(trans-4- methylcyclohexyl)-2- oxo-2-{[1-(tetrahydro- pyran-4-yl)pyrazol-4- yl]amino}ethyl]- benzamide | 10 | 516.2 | 1.51 |
| 30 | 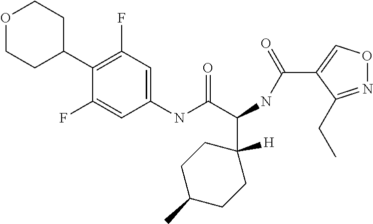 [SMILES: CCc1nocc1C(=O)N[C@H](C(=O)Nc1cc(F)c(C2CCOCC2)c(F)c1)[C@H]1CC[C@H](C)CC1] | N-{(1S)-2-[3,5- Difluoro-4-(tetrahydro- pyran-4-yl)anilino]-1- (trans-4-methyl- cyclohexyl)-2-oxo- ethyl}-3-ethyl- isoxazole-4- carboxamide | 8 | 490.0 | 2.78 |

### Example 31

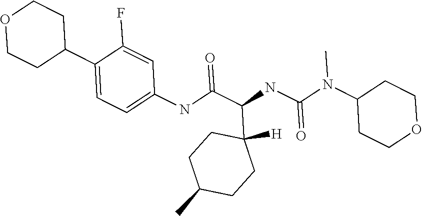 [SMILES: CN(C(=O)N[C@H](C(=O)Nc1ccc(C2CCOCC2)c(F)c1)[C@H]1CC[C@H](C)CC1)C1CCOCC1]

### (2S)—N-[3-Fluoro-4-(tetrahydropyran-4-yl)phenyl]-2-(trans-4-methylcyclohexyl)-2-{[methyl(tetrahydropyran-4-yl)carbamoyl]amino}acetamide

Intermediate 35 (33 mg, 0.095 mmol) was dissolved in DCM (1 mL) and treated with DIPEA (33 μL, 0.189 mmol), then N-methyl-N-(tetrahydropyran-4-yl)carbamoyl chloride (21 mg, 0.11 mmol) was added. The resulting mixture was stirred at r.t. for 18 h. DIPEA and N-methyl-N-(tetrahydropyran-4-yl)carbamoyl chloride (21 mg, 0.11 mmol) were each added repeatedly over the next 48 h, to bring the reaction to completion. The crude reaction mixture was purified by column chromatography, using a gradient of EtOAc/isohexane (0-100%), then MeOH/EtOAc (0-20%), to yield the title compound (25 mg, 21%), as a colourless glass. δ_{H} (400 MHz, DMSO-d₆) 10.15 (s, 1H), 7.61-7.51 (m, 1H), 7.31-7.18 (m, 2H), 6.10 (d, J 8.3 Hz, 1H), 4.15 (tt, J 11.8, 4.0 Hz, 1H), 4.05 (t, J 8.4 Hz, 1H), 3.90 (ddt, J 20.7, 10.6, 4.0 Hz, 4H), 3.48-3.34 (m, 4H), 2.98 (ddt, J 11.7, 7.8, 4.1 Hz, 1H), 2.71 (s, 3H), 1.83 (d, J 12.4 Hz, 1H), 1.77-1.55 (m, 8H), 1.50 (d, J 12.9 Hz, 1H), 1.40 (t, J 13.0 Hz, 3H), 1.23 (s, 1H), 1.18-1.04 (m, 1H), 1.02-0.88 (m, 1H), 0.85 (dd, J 8.5, 4.7 Hz, 5H). HPLC-MS (method 8): [M+H]⁺ m/z 490, RT 2.42 minutes.

### Example 32 (Procedure E)

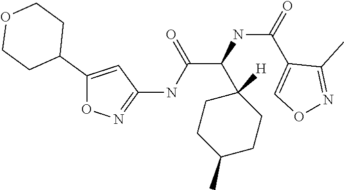 [SMILES: Cc1nocc1C(=O)N[C@H](C(=O)Nc1cc(C2CCOCC2)on1)[C@H]1CC[C@H](C)CC1]

### 3-Methyl-N-[(1S)-1-(trans-4-methylcyclohexyl)-2-oxo-2-{[5-(tetrahydropyran-4-yl)-isoxazol-3-yl]amino}ethyl]isoxazole-4-carboxamide

Intermediate 24 (21 mg, 0.077 mmol) was dissolved in DMF (0.4 mL, 5 mmol), then 5-(tetrahydropyran-4-yl)isoxazol-3-amine (14 mg, 0.079 mmol), DIPEA (20 μL, 0.12 mmol) and 2,4,6-tripropyl-1,3,5,2,4,6-trioxatriphosphorinane-2,4,6-trioxide solution (51 mg, 0.08 mmol) were added. The vial was sealed and the reaction mixture was heated at 70° C. for 18 h, then diluted with DCM (0.5 mL) and water (0.5 mL). The mixture was shaken and filtered through a PTFE phase separator (3 mL), then concentrated in vacuo. The crude residue was dissolved in MeOH (0.4 mL) and treated with HCl in 1,4-dioxane (4N, 0.5 mL). The reaction mixture was stirred at r.t. for 1 h, then concentrated in vacuo. The crude residue was suspended in DCM (1.0 mL) and treated with 3-methylisoxazole-4-carboxylic acid (10 mg, 0.08 mmol), HATU (75 mg, 0.2 mmol), and DIPEA (100 μL, 0.60 mmol). The reaction mixture was stirred at r.t. After 18 h, more HATU (50 mg, 0.13 mmol), and DIPEA (100 μL, 0.60 mmol) were added. The mixture was stirred at r.t. overnight, then purified by preparative HPLC (method 15), to yield the title compound (2.4 mg, 3%). HPLC-MS (method 13): [M+H]⁺ m/z 431, RT 2.59 minutes.

### Examples 33 to 37

The title compounds were prepared from Intermediate 24 and the appropriate commercially available heteroaryl amine according to Procedure E.

| | | | | | LCMS |
|---|---|---|---|---|---|
| | | | LCMS | LCMS | RT |
| Ex. | Structure | Method | Mass | Name | (min) |
| 33 | 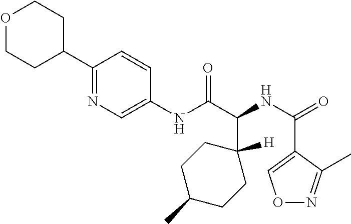 [SMILES: Cc1nocc1C(=O)N[C@H](C(=O)Nc1ccc(C2CCOCC2)nc1)[C@H]1CC[C@H](C)CC1] | 3-Methyl-N-[(1S)-1- (trans-4-methyl- cyclohexyl)-2-oxo-2- {[6-(tetrahydropyran-4- yl)pyridin-3-yl]- amino}ethyl]isoxazole- 4-carboxamide | 12 | 441.3 | 4.16 |
| 34 | 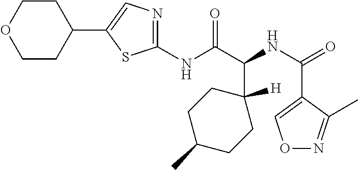 [SMILES: Cc1nocc1C(=O)N[C@H](C(=O)Nc1ncc(C2CCOCC2)s1)[C@H]1CC[C@H](C)CC1] | 3-Methyl-N-[(1S)-1- (trans-4-methyl- cyclohexyl)-2-oxo-2- {[5-(tetrahydropyran-4- yl)thiazol-2-yl]amino}- ethyl]isoxazole-4- carboxamide | 12 | 447.3 | 4.36 |
| 35 | 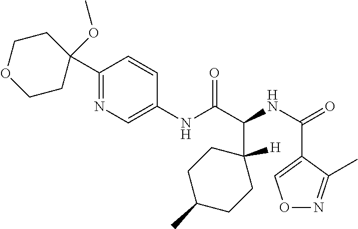 [SMILES: COC1(c2ccc(NC(=O)[C@@H](NC(=O)c3conc3C)[C@H]3CC[C@H](C)CC3)cn2)CCOCC1] | N-[(1S)-2-{[6-(4- Methoxytetrahydro- pyran-4-yl)pyridin-3- yl]amino}-1-(trans-4- methylcyclohexyl)-2- oxoethyl]-3-methyl- isoxazole-4- carboxamide | 12 | 471.3 | 4.23 |
| 36 | 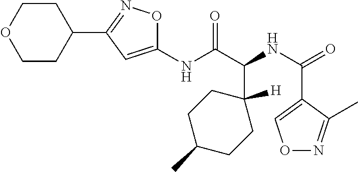 [SMILES: Cc1nocc1C(=O)N[C@H](C(=O)Nc1cc(C2CCOCC2)no1)[C@H]1CC[C@H](C)CC1] | 3-Methyl-N-[(1S)-1- (trans-4-methyl- cyclohexyl)-2-oxo-2- {[3-(tetrahydropyran-4- yl)isoxazol-5-yl]- amino}ethyl]isoxazole- 4-carboxamide | 12 | 431.3 | 4.27 |
| 37 | 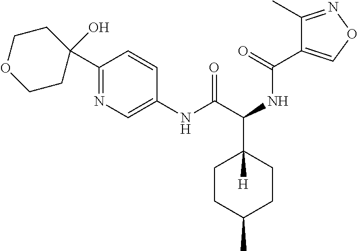 | N-[(1S)-2-{[6-(4- Hydroxytetrahydro- pyran-4-yl)pyridin-3- yl]amino}-1-(trans-4- methylcyclohexyl)-2- oxoethyl]-3-methyl- isoxazole-4- carboxamide | 12 | 457.3 | 3.93 |

### Example 38

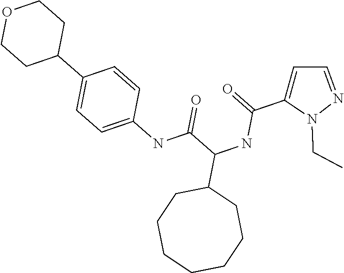 [SMILES: CCn1nccc1C(=O)NC(C(=O)Nc1ccc(C2CCOCC2)cc1)C1CCCCCCC1]

### N-{1-Cyclooctyl-2-oxo-2-[4-(tetrahydropyran-4-yl)anilino]ethyl}-2-ethylpyrazole-3-carboxamide

N-[4-(Tetrahydropyran-4-yl)phenyl]formamide (50 mg, 0.22 mmol) was dissolved in DCM (2 mL) and stirred at r.t., then treated with triethylamine (140 μL, 0.99 mmol). The reaction mixture was cooled to 0° C., and phosphorus oxychloride (35 μL, 0.37 mmol) was added. The reaction mixture was stirred at 0° C. for 15 minutes, then warmed to r.t. and stirred overnight, then quenched with water (2 mL) and DCM (1 mL). The layers were separated with a phase separating cartridge. The aqueous phase was extracted with DCM (1 mL). The organic layers were combined and concentrated in vacuo. The crude residue was dissolved in 2,2,2-trifluoroethanol (2 mL), and cyclooctanecarbaldehyde (31 mg, 0.21 mmol), 2-ethylpyrazole-3-carboxylic acid (30 mg, 0.21 mmol) and ammonia (7N in MeOH, 60 μL, 0.42 mmol) were added. The mixture was stirred at r.t. overnight, then concentrated in vacuo. The residue was purified by preparative HPLC (method 17) to yield the title compound (12 mg). HPLC-MS (method 12): [M+H]⁺ m/z 467.4, RT 5.06 minutes.

### Example 39

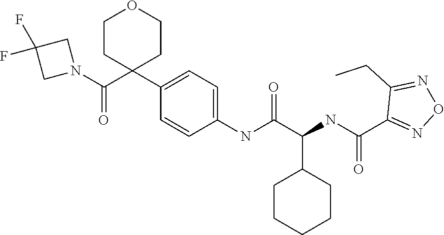 [SMILES: CCc1nonc1C(=O)N[C@H](C(=O)Nc1ccc(C2(C(=O)N3CC(F)(F)C3)CCOCC2)cc1)C1CCCCC1]

### N-[(1S)-1-Cyclohexyl-2-{4-[4-(3,3-difluoroazetidine-1-carbonyl)tetrahydropyran-4-yl]-anilino}-2-oxoethyl]-4-ethyl-1,2,5-oxadiazole-3-carboxamide

Intermediate 38 (125 mg, 0.29 mmol), 4-ethyl-1,2,5-oxadiazole-3-carboxylic acid (43 mg, 0.30 mmol) and HATU (118 mg, 0.30 mmol) were added to a round-bottomed flask, followed by DCM (10 mL). The mixture was stirred, then DIPEA (0.10 mL, 0.60 mmol) was added. The mixture was stirred for 18 h at room temperature, then concentrated in vacuo and purified by preparative HPLC (method 15), to afford, after freeze-drying, the title compound (55 mg, 34%) as a white solid. δ_{H} (300 MHz, DMSO-d₆) 10.32 (s, 1H), 9.16 (d, J 8.0 Hz, 1H), 7.66 (d, J 8.3 Hz, 2H), 7.26 (d, J 8.3 Hz, 2H), 4.48 (t, J 8.1 Hz, 1H), 4.12 (br s, 4H), 3.84-3.45 (m, 4H), 2.90 (q, J 7.5 Hz, 2H), 2.18 (d, J 13.6 Hz, 2H), 2.00-1.54 (m, 7H), 1.35-0.89 (m, 9H). LCMS (method 8): MH⁺ m/z 560.4, RT 2.14 minutes.

1. A compound of formula (I) or an N-oxide thereof, or a pharmaceutically acceptable salt thereof: 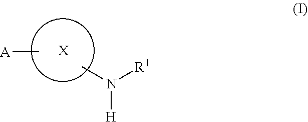
wherein
X represents an optionally substituted benzene ring; or an optionally substituted five-membered heteroaromatic ring selected from furyl, thienyl, pyrrolyl, pyrazolyl, oxazolyl, isoxazolyl, thiazolyl, isothiazolyl and imidazolyl; or an optionally substituted six-membered heteroaromatic ring selected from pyridinyl, pyridazinyl, pyrimidinyl and pyrazinyl;
A represents C₃₋₉ cycloalkyl, C₃₋₇ heterocycloalkyl or C₄₋₉ heterobicycloalkyl, any of which groups may be optionally substituted by one or more substituents;
R¹ represents —COR^{a} or —SO₂R^{b}; or R¹ represents C₁₋₆ alkyl, C₃₋₉ cycloalkyl, C₃₋₉ cycloalkyl(C₁₋₆)alkyl, C₅₋₉ spirocycloalkyl(C₁₋₆)alkyl, aryl, aryl(C₁₋₆)alkyl, C₃₋₇ heterocycloalkyl, C₃₋₇ heterocycloalkyl(C₁₋₆)alkyl, heteroaryl or heteroaryl(C₁₋₆)alkyl, any of which groups may be optionally substituted by one or more substituents;
R^{a} represents hydrogen; or R^{a} represents C₁₋₆ alkyl, C₂₋₇ alkenyl, C₃₋₉ cycloalkyl, C₃₋₉ cycloalkyl(C₁₋₆)alkyl, C₃₋₉ cycloalkylidenyl(C₁₋₆)alkyl, C₄₋₉ bicycloalkyl(C₁₋₆)alkyl, C₄₋₉ bicycloalkylidenyl(C₁₋₆)alkyl, C₅₋₉ spirocycloalkyl(C₁₋₆)alkyl, C₉₋₁₁ tricycloalkyl-(C₁₋₆)alkyl, aryl, aryl(C₁₋₆)alkyl, C₃₋₇ heterocycloalkyl, C₃₋₇ heterocycloalkyl(C₁₋₆)alkyl, C₃₋₇ heterocycloalkylidenyl(C₁₋₆)alkyl, heteroaryl or heteroaryl(C₁₋₆)alkyl, any of which groups may be optionally substituted by one or more substituents; and
R^{b} represents C₁₋₆ alkyl, C₂₋₇ alkenyl, C₃₋₉ cycloalkyl, C₃₋₉ cycloalkyl(C₁₋₆)alkyl, C₃₋₉ cycloalkylidenyl(C₁₋₆)alkyl, C₄₋₉ bicycloalkyl(C₁₋₆)alkyl, C₄₋₉ bicycloalkylidenyl-(C₁₋₆)alkyl, C₅₋₉ spirocycloalkyl(C₁₋₆)alkyl, C₉₋₁₁ tricycloalkyl(C₁₋₆)alkyl, aryl, aryl(C₁₋₆)-alkyl, C₃₋₇ heterocycloalkyl, C₃₋₇ heterocycloalkyl(C₁₋₆)alkyl, C₃₋₇ heterocycloalkylidenyl-(C₁₋₆)alkyl, heteroaryl or heteroaryl(C₁₋₆)alkyl, any of which groups may be optionally substituted by one or more substituents.

2. A compound as claimed in claim 1 wherein X represents an optionally substituted benzene ring; or an optionally substituted five-membered heteroaromatic ring selected from pyrazolyl, isoxazolyl and thiazolyl; or an optionally substituted six-membered heteroaromatic ring selected from pyridinyl; wherein the optional substituents on X include one, two or three substituents independently selected from halogen, C₁₋₆ alkyl and C₁₋₆ alkoxy.

3. A compound as claimed in claim 1 wherein A represents tetrahydrofuranyl, pyrrolidinyl, tetrahydropyranyl or morpholinyl, any of which groups may be optionally substituted by one, two or three substituents independently selected from cyano, hydroxy, hydroxy(C₁₋₆)alkyl, oxo, C₁₋₆ alkoxy, di(C₁₋₆)alkylaminocarbonyl and difluoroazetidinylcarbonyl.

4. A compound as claimed in claim 1 wherein R¹ represents —COR^{a}, in which R^{a} is as defined in claim 1.

5. A compound as claimed in claim 4 wherein R^{a} represents —CH(R⁵)N(H)C(O)R⁶, —CH(R⁵)N(H)S(O)₂R⁶, —C(═CR^{5a}R^{5b})N(H)C(O)R⁶, —CH(R⁵)R⁷, —CH(R⁵)N(H)R⁷ or
—CH(R⁵)C(O)N(H)R⁷, in which
R⁵ represents hydrogen; or R⁵ represents C₁₋₅ alkyl, C₃₋₉ cycloalkyl, C₃₋₉ cyclo-alkyl(C₁₋₅)alkyl, C₄₋₉ bicycloalkyl, C₄₋₉ bicycloalkyl(C₁₋₅)alkyl, C₅₋₉ spirocycloalkyl, C₅₋₉ spirocycloalkyl(C₁₋₅)alkyl, C₉₋₁₁ tricycloalkyl, C₉₋₁₁ tricycloalkyl(C₁₋₅)alkyl, aryl, aryl-(C₁₋₅)alkyl, C₃₋₇ heterocycloalkyl, C₃₋₇ heterocycloalkyl(C₁₋₅)alkyl, heteroaryl or heteroaryl(C₁₋₅)alkyl, any of which groups may be optionally substituted by one or more substituents;
R^{5a} represents C₃₋₇ cycloalkyl, C₄₋₉ bicycloalkyl, aryl, C₃₋₇ heterocycloalkyl or heteroaryl, any of which groups may be optionally substituted by one or more substituents; and
R^{5b} represents hydrogen or C₁₋₆ alkyl; or
R^{5a} and R^{5b}, when taken together with the carbon atom to which they are both attached, represent C₃₋₇ cycloalkyl, C₄₋₉ bicycloalkyl or C₃₋₇ heterocycloalkyl, any of which groups may be optionally substituted by one or more substituents;
R⁶ represents —NR^{6a}R^{6b} or —OR^{6c}; or R⁶ represents C₁₋₉ alkyl, C₃₋₉ cycloalkyl, C₃₋₉ cycloalkyl(C₁₋₆)alkyl, aryl, aryl(C₁₋₆)alkyl, C₃₋₇ heterocycloalkyl, C₃₋₇ heterocycloalkyl-(C₁₋₆)alkyl, heteroaryl, heteroaryl(C₁₋₆)alkyl or spiro[(C₃₋₇)heterocycloalkyl][heteroaryl], any of which groups may be optionally substituted by one or more substituents;
R^{6a} represents hydrogen; or R^{6a} represents C₁₋₆ alkyl, C₃₋₇ cycloalkyl, C₃₋₇ cyclo-alkyl(C₁₋₆)alkyl, aryl, aryl(C₁₋₆)alkyl, C₃₋₇ heterocycloalkyl, C₃₋₇ heterocycloalkyl(C₁₋₆)-alkyl, heteroaryl, heteroaryl(C₁₋₆)alkyl or spiro[(C₃₋₇)heterocycloalkyl][heteroaryl], any of which groups may be optionally substituted by one or more substituents;
R^{6b} represents hydrogen or C₁₋₆ alkyl;
R^{6c} represents C₁₋₆ alkyl, C₃₋₇ cycloalkyl, C₃₋₇ cycloalkyl(C₁₋₆)alkyl, aryl, aryl(C₁₋₆)alkyl, C₃₋₇ heterocycloalkyl, C₃₋₇ heterocycloalkyl(C₁₋₆)alkyl, heteroaryl or heteroaryl(C₁₋₆)alkyl, any of which groups may be optionally substituted by one or more substituents; and
R⁷ represents aryl, heteroaryl or spiro[(C₃₋₇)heterocycloalkyl][heteroaryl], any of which groups may be optionally substituted by one or more substituents.

6. A compound as claimed in claim 5 represented by formula (IIA), or a pharmaceutically acceptable salt thereof: 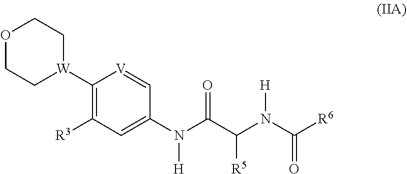
wherein
V represents N or C—R²;
W represents N or C—R¹¹;
R² represents hydrogen, halogen, cyano, C₁₋₆ alkyl, fluoromethyl, difluoromethyl, trifluoromethyl, hydroxy, C₁₋₆ alkoxy, difluoromethoxy, trifluoromethoxy, C₁₋₆ alkylthio, C₁₋₆ alkylsulfinyl, C₁₋₆ alkylsulfonyl, amino, C₁₋₆ alkylamino, di(C₁₋₆)alkylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl or di(C₁₋₆)alkylaminosulfonyl;
R³ represents hydrogen, halogen, C₁₋₆ alkyl or C₁₋₆ alkoxy;
R¹¹ represents hydrogen, C₁₋₆ alkyl, halogen, cyano, trifluoromethyl, hydroxy, hydroxy(C₁₋₆)alkyl, C₁₋₆ alkoxy, C₁₋₆ alkylthio, C₁₋₆ alkylsulfinyl, C₁₋₆ alkylsulfonyl, C₂₋₆ alkylcarbonyl, amino, C₁₋₆ alkylamino, di(C₁₋₆)alkylamino, aminocarbonyl, C₁₋₆ alkylaminocarbonyl, di(C₁₋₆)alkylaminocarbonyl or difluoroazetidinylcarbonyl; and
R⁵ and R⁶ are as defined in claim 5.

7. A compound as claimed in claim 5 wherein R⁵ represents C₁₋₅ alkyl, C₃₋₉ cycloalkyl, C₃₋₉ cycloalkyl(C₁₋₅)alkyl, C₄₋₉ bicycloalkyl, C₄₋₉ bicycloalkyl(C₁₋₅)alkyl, C₅₋₉ spirocycloalkyl, C₉₋₁₁ tricycloalkyl, C₉₋₁₁ tricycloalkyl(C₁₋₅)alkyl, aryl, aryl(C₁₋₅)alkyl, C₃₋₇ heterocycloalkyl, C₃₋₇ heterocycloalkyl(C₁₋₅)alkyl or heteroaryl(C₁₋₅)alkyl, any of which groups may be optionally substituted by one, two or three substituents independently selected from halogen, cyano, C₁₋₆ alkyl, trifluoromethyl, phenyl, hydroxy, C₁₋₆ alkoxy and aminocarbonyl.

8. A compound as claimed in claim 5 wherein R⁶ represents
—NR^{6a}R^{6b} or —OR^{6c}; or R⁶ represents C₁₋₉ alkyl, aryl, C₃₋₇ heterocycloalkyl, heteroaryl, heteroaryl(C₁₋₆)alkyl or spiro[(C₃₋₇)heterocycloalkyl][heteroaryl], any of which groups may be optionally substituted by one, two or three substituents independently selected from halogen, cyano, nitro, C₁₋₆ alkyl, difluoromethyl, trifluoromethyl, difluoroethyl, trifluoroethyl, trifluoropropyl, cyclopropyl, cyclobutyl, cyclopropylmethyl, phenyl, fluorophenyl, hydroxy, hydroxy(C₁₋₆)alkyl, oxo, C₁₋₆ alkoxy, C₁₋₆ alkoxy(C₁₋₆)alkyl, difluoromethoxy, trifluoromethoxy, C₁₋₆ alkylthio, C₁₋₆ alkylsulfinyl, C₁₋₆ alkylsulfonyl, 6)alkylsulfonyl(C₁₋₆)alkyl, C₁₋₆ alkylsulfonyloxy, amino, amino(C₁₋₆)alkyl, C₁₋₆ alkylamino, di(C₁₋₆)alkylamino, di(C₁₋₆)alkylamino(C₁₋₆)alkyl, pyrrolidinyl, dioxoisothiazolidinyl, tetrahydropyranyl, morpholinyl, piperazinyl, C₂₋₆ alkylcarbonylamino,
C₂₋₆ alkylcarbonylamino(C₁₋₆)alkyl, C₂₋₆ alkoxycarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl, 6)alkylaminosulfonyl and di(C₁₋₆)alkylsulfoximinyl.

9. A compound as claimed in claim 1 which is
N-{2-[3-Chloro-4-(morpholin-4-yl)anilino]-1-cyclooctyl-2-oxoethyl}-3-methylisoxazole-4-carboxamide;
N-[4-(4-Cyanotetrahydropyran-4-yl)-3-methylphenyl]-2-cyclooctyl-2-[(3-methylisoxazol-4-yl)formamido]acetamide;
4-(4-{2-Cyclooctyl-2-[(3-methylisoxazol-4-yl)formamido]acetamido}-2-methylphenyl)-N,N-dimethyltetrahydropyran-4-carboxamide;
N-{1-Cyclooctyl-2[3-methyl-4-(morpholin-4-yl)anilino]-2-oxo-ethyl}-3-methyl-isoxazole-4-carboxamide;
N-{1-Cyclooctyl-2-[3-fluoro-4-(morpholin-4-yl)anilino]-2-oxo-ethyl}-3-methyl-isoxazole-4-carboxamide;
N-(1-Cyclooctyl-2-{[6-(morpholin-4-yl)-pyridin-3-yl]amino}-2-oxoethyl)-3-methyl-isoxazole-4-carboxamide;
N-{1-Cyclooctyl-2-oxo-2-[4-(tetrahydro-pyran-4-yl)anilino]-ethyl}-3-methyl-isoxazole-4-carboxamide;
N-{1-Cyclooctyl-2-oxo-2[4-(tetrahydro-furan-3-yl)anilino]ethyl}-3-methyl-isoxazole-4-carboxamide;
N-{1-Cyclooctyl-2-oxo-2[4-(5-oxo-pyrrolidin-2-yl)-anilino]ethyl}-3-methylisoxazole-4-carboxamide;
N-(1-Cyclooctyl-2-oxo-2-{[5-(tetrahydropyran-4-yl)isoxazol-3-yl]-amino}ethyl)-3-methylisoxazole-4-carboxamide;
N-(1-Cyclooctyl-2-{[4-methyl-3-(tetrahydro-pyran-4-yl)isoxazol-5-yl]amino}-2-oxoethyl)-3-methylisoxazole-4-carboxamide;
N-{1-Cyclooctyl-2[3-methoxy-4-(morpholin-4-yl)anilino]-2-oxo-ethyl}-3-methyl-isoxazole-4-carboxamide;
N-{1-Cyclooctyl-2[3,5-difluoro-4-(morpholin-4-yl)-anilino]-2-oxoethyl}-3-methylisoxazole-4-carboxamide;
N-(2-{[5-Chloro-6-(morpholin-4-yl)-pyridin-3-yl]amino}-1-cyclooctyl-2-oxoethyl)-3-methylisoxazole-4-carboxamide;
N-(1-Cyclooctyl-2{[5-fluoro-6-(morpholin-4-yl)pyridin-3-yl]-amino}-2-oxoethyl)-3-methylisoxazole-4-carboxamide;
N-{1-Cyclooctyl-2[4-(4-hydroxytetrahydro-pyran-4-yl)anilino]-2-oxoethyl}-3-methyl-isoxazole-4-carboxamide;
N-{2-[4-(4-Cyano-tetrahydropyran-4-yl)-anilino]-1-cyclooctyl-2-oxoethyl}-3-methyl-isoxazole-4-carboxamide;
N-(1-Cyclooctyl-2-oxo-2-{[5-(tetrahydropyran-4-yl)pyridin-2-yl]-amino}ethyl)-3-methylisoxazole-4-carboxamide;
4-(4-{2-Cyclooctyl-2-[(3-methylisoxazol-4-yl)formamido]-acetamido}phenyl)-N,N-dimethyl-tetrahydropyran-4-carboxamide;
2-[(7Z)-5-Chlorobicyclo[4.2.0]octa-1,3,5-trien-7-ylidene]-2-[(3-methylisoxazol-4-yl)-formamido]-N-[4-(tetrahydropyran-4-yl)phenyl]acetamide;
3-Ethyl-N-{(1 S)-1-(trans-4-methylcyclohexyl)-2-oxo-2[4-(tetrahydropyran-4-yl)anilino]-ethyl}isoxazole-4-carboxamide;
3-Ethyl-N-{(1S)-2-[3-fluoro-4-(tetrahydro-pyran-4-yl)anilino]-1-(trans-4-methylcyclo-hexyl)-2-oxoethyl}-isoxazole-4-carboxamide;
N-{1-Cyclooctyl-2-oxo-2-[4-(tetrahydro-pyran-4-yl)anilino]-ethyl}-3-ethyl-isoxazole-4-carboxamide;
N-{1-Cyclooctyl-2-oxo-2-[3-(tetrahydro-pyran-4-yl)anilino]-ethyl}-3-methyl-isoxazole-4-carboxamide;
N-(1-Cyclooctyl-2-{4-[4-(hydroxymethyl)-tetrahydropyran-4-yl]-anilino}-2-oxoethyl)-3-methylisoxazole-4-carboxamide;
N-{2-[3-Bromo-4-(morpholin-4-yl)-anilino]-1-cyclooctyl-2-oxoethyl}-3-methyl-isoxazole-4-carboxamide;
N-{(1S)-1-(4-Methyl-cyclohexyl)-2-oxo-2[4-(tetrahydropyran-4-yl)anilino]ethyl}-3-(methylsulfonyl-methyl)benzamide;
3-(Methane-sulfonamido)-N-{(1S)-1-(trans-4-methyl-cyclohexyl)-2-oxo-2[4-(tetrahydropyran-4-yl)anilino]ethyl}benzamide;
3-{[Dimethyl(oxo)-λ⁶-sulfanylidene]amino}-N-[(1S)-1-(trans-4-methylcyclohexyl)-2-oxo-2-{[1-(tetrahydro-pyran-4-yl)pyrazol-4-yl]amino}ethyl]benzamide;
N-{(1S)-2[3,5-Difluoro-4-(tetrahydro-pyran-4-yl)anilino]-1-(trans-4-methyl-cyclohexyl)-2-oxo-ethyl}-3-ethyl-isoxazole-4-carboxamide;
(2S)—N-[3-Fluoro-4-(tetrahydropyran-4-yl)phenyl]-2-(trans-4-methylcyclohexyl)-2-{[methyl(tetrahydropyran-4-yl)carbamoyl]amino}acetamide;
3-Methyl-N-[(1 S)-1-(trans-4-methylcyclohexyl)-2-oxo-2-{[5-(tetrahydropyran-4-yl)-isoxazol-3-yl]amino}ethyl]isoxazole-4-carboxamide;
3-Methyl-N-[(1 S)-1-(trans-4-methyl-cyclohexyl)-2-oxo-2{[6-(tetrahydropyran-4-yl)pyridin-3-yl]-amino}ethyl]isoxazole-4-carboxamide;
3-Methyl-N-[(1S)-1-(trans-4-methyl-cyclohexyl)-2-oxo-2{[5-(tetrahydropyran-4-yl)thiazol-2-yl]amino}ethyl]isoxazole-4-carboxamide;
N-[(1S)-2{[6-(4-Methoxytetrahydro-pyran-4-yl)pyridin-3-yl]amino}-1-(trans-4-methylcyclohexyl)-2-oxoethyl]-3-methyl-isoxazole-4-carboxamide;
3-Methyl-N-[(1S)-1-(trans-4-methyl-cyclohexyl)-2-oxo-2{[3-(tetrahydropyran-4-yl)isoxazol-5-yl]amino}ethyl]isoxazole-4-carboxamide;
N-[(1S)-2-{[6-(4-Hydroxytetrahydro-pyran-4-yl)pyridin-3-yl]amino}-1-(trans-4-methylcyclohexyl)-2-oxoethyl]-3-methyl-isoxazole-4-carboxamide;
N-{1-Cyclooctyl-2-oxo-2-[4-(tetrahydropyran-4-yl)anilino]ethyl}-2-ethylpyrazole-3-carboxamide; or
N-[(1S)-1-Cyclohexyl-2-{4-[4-(3,3-difluoroazetidine-1-carbonyl)tetrahydropyran-4-yl]-anilino}-2-oxoethyl]-4-ethyl-1,2,5-oxadiazole-3-carboxamide.

10-12. (canceled)

13. A pharmaceutical composition comprising a compound of formula (I) as defined in claim 1 or an N-oxide thereof, or a pharmaceutically acceptable salt thereof, in association with a pharmaceutically acceptable carrier.

14. A pharmaceutical composition as claimed in claim 13 further comprising an additional pharmaceutically active ingredient.

15-16. (canceled)

17. A method for the treatment and/or prevention of disorders for which the administration of a modulator of IL-17 function is indicated which comprises administering to a patient in need of such treatment an effective amount of a compound of formula (I) as defined in claim 1 or an N-oxide thereof, or a pharmaceutically acceptable salt thereof.

18. A method for the treatment and/or prevention of an inflammatory or autoimmune disorder, which comprises administering to a patient in need of such treatment an effective amount of a compound of formula (I) as defined in claim 1 or an N-oxide thereof, or a pharmaceutically acceptable salt thereof.

19. A compound as claimed in claim 6 wherein R⁵ represents C₁₋₅ alkyl, C₃₋₉ cycloalkyl, C₃₋₉ cycloalkyl(C₁₋₅)alkyl, C₄₋₉ bicycloalkyl, C₄₋₉ bicycloalkyl(C₁₋₅)alkyl, C₅₋₉ spirocycloalkyl, C₉₋₁₁ tricycloalkyl, C₉₋₁₁ tricycloalkyl(C₁₋₅)alkyl, aryl, aryl(C₁₋₅)alkyl, C₃₋₇ heterocycloalkyl, C₃₋₇ heterocycloalkyl(C₁₋₅)alkyl or heteroaryl(C₁₋₅)alkyl, any of which groups may be optionally substituted by one, two or three substituents independently selected from halogen, cyano, C₁₋₆ alkyl, trifluoromethyl, phenyl, hydroxy, C₁₋₆ alkoxy and aminocarbonyl.

20. A compound as claimed in claim 6 wherein R⁶ represents —NR^{6a}R^{6b} or —OR^{6c}; or R⁶ represents C₁₋₉ alkyl, aryl, C₃₋₇ heterocycloalkyl, heteroaryl, heteroaryl(C₁₋₆)alkyl or spiro[(C₃₋₇)heterocycloalkyl][heteroaryl], any of which groups may be optionally substituted by one, two or three substituents independently selected from halogen, cyano, nitro, C₁₋₆ alkyl, difluoromethyl, trifluoromethyl, difluoroethyl, trifluoroethyl, trifluoropropyl, cyclopropyl, cyclobutyl, cyclopropylmethyl, phenyl, fluorophenyl, hydroxy, hydroxy(C₁₋₆)alkyl, oxo, C₁₋₆ alkoxy, C₁₋₆ alkoxy(C₁₋₆)alkyl, difluoromethoxy, trifluoromethoxy, C₁₋₆ alkylthio, C₁₋₆ alkylsulfinyl, C₁₋₆ alkylsulfonyl, 6)alkylsulfonyl(C₁₋₆)alkyl, C₁₋₆ alkylsulfonyloxy, amino, amino(C₁₋₆)alkyl, C₁₋₆ alkylamino, di(C₁₋₆)alkylamino, di(C₁₋₆)alkylamino(C₁₋₆)alkyl, pyrrolidinyl, dioxoisothiazolidinyl, tetrahydropyranyl, morpholinyl, piperazinyl, C₂₋₆ alkylcarbonylamino,
C₂₋₆ alkylcarbonylamino(C₁₋₆)alkyl, C₂₋₆ alkoxycarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl, 6)alkylaminosulfonyl and di(C₁₋₆)alkylsulfoximinyl.

21. A compound as claimed in claim 7 wherein R⁶ represents —NR^{6a}R^{6b} or —OR^{6c}; or R⁶ represents C₁₋₉ alkyl, aryl, C₃₋₇ heterocycloalkyl, heteroaryl, heteroaryl(C₁₋₆)alkyl or spiro[(C₃₋₇)heterocycloalkyl][heteroaryl], any of which groups may be optionally substituted by one, two or three substituents independently selected from halogen, cyano, nitro, C₁₋₆ alkyl, difluoromethyl, trifluoromethyl, difluoroethyl, trifluoroethyl, trifluoropropyl, cyclopropyl, cyclobutyl, cyclopropylmethyl, phenyl, fluorophenyl, hydroxy, hydroxy(C₁₋₆)alkyl, oxo, C₁₋₆ alkoxy, C₁₋₆ alkoxy(C₁₋₆)alkyl, difluoromethoxy, trifluoromethoxy, C₁₋₆ alkylthio, C₁₋₆ alkylsulfinyl, C₁₋₆ alkylsulfonyl, 6)alkylsulfonyl(C₁₋₆)alkyl, C₁₋₆ alkylsulfonyloxy, amino, amino(C₁₋₆)alkyl, C₁₋₆ alkylamino, di(C₁₋₆)alkylamino, di(C₁₋₆)alkylamino(C₁₋₆)alkyl, pyrrolidinyl, dioxoisothiazolidinyl, tetrahydropyranyl, morpholinyl, piperazinyl, C₂₋₆ alkylcarbonylamino,
C₂₋₆ alkylcarbonylamino(C₁₋₆)alkyl, C₂₋₆ alkoxycarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl, di(C₁₋₆) alkylaminosulfonyl and di(C₁₋₆)alkylsulfoximinyl.
